(19) United States
(12) Patent Application Publication
(10) Pub. No.: US 2007/0134218 A1
(43) Pub. Date: Jun. 14, 2007

(54) Methods of inducing or increasing the expression of proteoglycans such as aggrecan in cells

(76) Inventors: William F. McKay, Memphis, TN (US); Scott D. Boden, Atlanta, GA (US); Sangwook T. Yoon, Atlanta, GA (US)

Correspondence Address: FOX ROTHSCHILD, LLP, 997 LENOX DRIVE, LAWRENCEVILLE, NJ, 08648, US

(21) Appl. No.: 11/602,805

(22) Filed: Nov. 21, 2006

Related U.S. Application Data
(63) Continuation-in-part of application No. 10/382,844, filed on Mar. 7, 2003, now abandoned.
Application No. 10/382,844 is a continuation-in-part of application No. 10/292,951, filed on Nov. 13, 2002.
(60) Provisional application No. 60/331,321, filed on Nov. 14, 2001.

(51) Int. Cl. A61K 48/00

(52) U.S. Cl. 424/93.21; 514/44

(57) ABSTRACT

Methods of inducing the expression of a proteoglycan such as aggrecan in a cell are described. A method is described which includes transfecting a cell with an isolated nucleic acid comprising a nucleotide sequence encoding a LIM mineralization protein operably linked to a promoter. The LIM mineralization protein can be rLMP, hLMP-1, hLMP-1s, or hLMP-3. Transfection maybe accomplished ex vivo or in vivo by direct injection of virus or naked DNA, or by a nonviral vector such as a plasmid. The method can be used to induce proteoglycan synthesis in osseous cells or to stimulate proteoglycan and/or collagen production in cells capable of producing proteoglycan and/or collagen (e.g., intervertebral disc cells including cells of the nucleus pulposus and annulus fibrosus).

This application is a continuation-in-part of U.S. patent application Ser. No. 10/382,844, filed Mar. 7, 2003, pending, which is continuation-in-part of U.S. patent application Ser. No. 10/292,951, filed Nov. 13, 2002, pending, which application claims priority to U.S. Provisional Application Ser. No. 60/331,321, filed Nov. 14, 2001. Each of these applications is incorporated herein by reference in its entirety.

This application is related to U.S. patent application Ser. No. 09/124,238, filed Jul. 29, 1998, now U.S. Pat. No. 6,300,127, and U.S. patent application Ser. No. 09/959,578, filed Apr. 28, 2000, pending. Each of these applications is incorporated by reference herein in its entirety.

### FIELD OF THE INVENTION

The field of the invention relates generally to methods for transfecting cells with genetic material. More specifically, the field of the invention relates to methods of inducing or increasing the expression of a proteoglycan such as aggrecan in a cell by transfecting the cell with a nucleic acid encoding a LIM mineralization protein (LMP).

### BACKGROUND OF THE INVENTION

Osteoblasts are thought to differentiate from pluripotent mesenchymal stem cells. The maturation of an osteoblast results in the secretion of an extracellular matrix which can mineralize and form bone. The regulation of this complex process is not well understood but is thought to involve a group of signaling glycoproteins known as bone morphogenetic proteins (BMPS). These proteins have been shown to be involved with embryonic dorsal-ventral patterning, limb bud development, and fracture repair in adult animals. B. L. Hogan, Genes & Develop., 10, 1580 (1996). This group of transforming growth factor-beta superfamily secreted proteins has a spectrum of activities in a variety of cell types at different stages of differentiation; differences in physiological activity between; these closely related molecules have not been clarified. D. M. Kingsley, Trends Genet., 10, 16 (1994).

To better discern the unique physiological role of different BMP signaling proteins, we recently compared the potency of BMP-6 with that of BMP-2 and BMP-4, for inducing rat calvarial osteoblast differentiation. Boden, et al., Endocrinology, 137, 3401 (1996). We studied this process in first passage (secondary) cultures of fetal rat calvaria that require BMP or glucocorticoid for initiation of differentiation. In this model of membranous bone formation, glucocorticoid (GC) or a BMP will initiate differentiation to mineralized bone nodules capable of secreting osteocalcin, the osteoblast-specific protein. This secondary culture system is distinct from primary rat osteoblast cultures which undergo spontaneous differentiation. In this secondary system, glucocorticoid resulted in a ten-fold induction of BMP-6 mRNA and protein expression which was responsible for the enhancement of osteoblast differentiation. Boden, et al., Endocrinology, 138, 2920 (1997).

In addition to extracellular signals, such as the BMPs, intracellular signals or regulatory molecules may also play a role in the cascade of events leading to formation of new bone. One broad class of intracellular regulatory molecules are the LIM proteins, which are so named because they possess a characteristic structural motif known as the LIM domain. The LIM domain is a cysteine-rich structural motif composed of two special zinc fingers that are joined by a 2-amino acid spacer. Some proteins have only LIM domains, while others contain a variety of additional functional domains. LIM proteins form a diverse group, which includes transcription factors and cytoskeletal proteins. The primary role of LIM domains appears to be in mediating protein-protein interactions, through the formation of dimers with identical or different LIM domains, or by binding distinct proteins.

In LIM homeodomain proteins, that is, proteins having both LIM domains and a homeodomain sequence, the LIM domains function as negative regulatory elements. LIM homeodomain proteins are involved in the control of cell lineage determination and the regulation of differentiation, although LIM-only proteins may have similar roles. LIM-only proteins are also implicated in the control of cell proliferation since several genes encoding such proteins are associated with oncogenic chromosome translocations.

Humans and other mammalian species are prone to diseases or injuries that require the processes of bone repair and/or regeneration. For example, treatment of fractures would be improved by new treatment regimens that could stimulate the natural bone repair mechanisms, thereby reducing the time required for the fractured bone to heal. In another example, individuals afflicted with systemic bone disorders, such as osteoporosis, would benefit from treatment regimens that would results in systemic formation of new bone. Such treatment regimens would reduce the incidence of fractures arising from the loss of bone mass that is a characteristic of this disease.

For at least these reasons, extracellular factors, such as the BMPs, have been investigated for the purpose of using them to stimulate formation of new bone in vivo. Despite the early successes achieved with BMPs and other extracellular signalling molecules, their use entails a number of disadvantages. For example, relatively large doses of purified BMPs are required to enhance the production of new bone, thereby increasing the expense of such treatment methods. Furthermore, extracellular proteins are susceptible to degradation following their introduction into a host animal. In addition, because they are typically immunogenic, the possibility of stimulating an immune response to the administered proteins is ever present.

Due to such concerns, it would be desirable to have available treatment regimens that use an intracellular signaling molecule to induce new bone formation. Advances in the field of gene therapy now make it possible to introduce into osteogenic precursor cells, that is, cells involved in bone formation, or peripheral blood leukocytes, nucleotide fragments encoding intracellular signals that form part of the bone formation process. Gene therapy for bone formation offers a number of potential advantages: (1) lower production costs; (2) greater efficacy, compared to extracellular treatment regiments, due to the ability to achieve prolonged expression of the intracellular signal; (3) it would by-pass the possibility that treatment with extracellular signals might be hampered due to the presence of limiting numbers of receptors for those signals; (4) it permits the delivery of transfected potential osteoprogenitor cells directly to the site where localized bone formation is required; and (5) it would permit systemic bone formation, thereby providing a treatment regimen for osteoporosis and other metabolic bone diseases.

In addition to diseases of the bone, humans and other mammalian species are also subject to intervertebral disc degeneration, which is associated with, among other things, low back pain, disc herniations, and spinal stenosis. Disc degeneration is associated with a progressive loss of proteoglycan matrix. This may cause the disc to be more susceptible to bio-mechanical injury and degeneration. Accordingly, it would be desirable to have a method of stimulating proteoglycan and/or collagen synthesis by the appropriate cells, such as, for example, cells of the nucleous pulposus, cells of the annulus fibrosus, and cells of the intervertebral disc.

Additionally, there still exists a need to develop a better understanding of the mechanisms of LMP action in the induction of bone formation. By gaining a better understanding of the intracellular signaling pathways involved with osteoblast differentiation, bone formation in a clinical setting could be improved.

### SUMMARY OF THE INVENTION

According to a first aspect of the invention, a method of inducing or increasing the expression of a proteoglycan in a cell is provided. The method includes transfecting a cell with an isolated nucleic acid comprising a nucleotide sequence encoding a LIM mineralization protein operably linked to a promoter. The expression of aggrecan can be induced or increased according to this aspect of the invention. The isolated nucleic acid can be a nucleic acid which can hybridize under standard conditions to a nucleic acid molecule complementary to the full length of SEQ. ID NO: 25; and/or a nucleic acid molecule which can hybridize under highly stringent conditions to a nucleic acid molecule complementary to the full length of SEQ. ID NO: 26. The cell can be any somatic cell such including, but not limited to, buffy coat cells, stem cells and intervertebral disc cells. The isolated nucleic acid can be in a vector such as an adenovirus vector.

According to a second aspect of the invention, a cell which overexpresses a proteoglycan is provided. According to this aspect of the invention, the cell can be a cell which overexpresses aggrecan. The cell can be a buffy coat cell, an intervertebral disc cell, a mesenchymal stem cell or a pluripotential stem cell. An implant comprising a cell as set forth above and a carrier material is also provided. Also provided according to the invention is a method of treating intervertebral disc disease in a mammal comprising introducing a cell as set forth above into an intervertebral disc of the mammal.

Additional advantages and novel features of the invention will be set forth in part in the description that follows, and in part will become more apparent to those skilled in the art upon examination of the following or upon learning by practice of the invention.

### BRIEF DESCRIPTION OF THE DRAWINGS

The present invention may be better understood with reference to the accompanying drawings in which:

FIG. 1 is a graph showing the production of sulfated glycosaminoglycan (sGAG) after expression of HLMP-1 by rat intervertebral disc cells transfected with different MOIs;

FIG. 2 is a chart showing the dose response of rat intervertebral disc cells six days after infection with different MOI of AdHLMP-1;

FIG. 3 is a chart showing the expression of Aggrecan and BMP-2 mRNA by AdHLMP-1 transfected rat intervertebral disc cells six days following transfection with an MOI of 250 virions/cell;

FIG. 4A is a chart showing HLMP-1 mRNA expression 12 hours after infection with Ad-hLMP-1 at different MOIs;

FIG. 4B is a chart showing the production of sGAG in medium from 3 to 6 days after infection;

FIG. 5 is a chart showing time course changes of the production of sGAG; FIG. 6A is a chart showing gene response to LMP-1 over-expression in rat annulus fibrosus cells for aggrecan

FIG. 6B is a chart showing gene response to LMP-1 over-expression in rat annulus fibrosus cells for BMP-2;

FIG. 7 is a graph showing the time course of HLMP-1 mRNA levels in rat annulus fibrosus cells after infection with AdLMP-1 at MOI of 25;

FIG. 8 is a chart showing changes in mRNA levels of BMPs and aggrecan in response to HLMP-1 over-expression;

FIG. 9 is a graph showing the time course of sGAG production enhancement in response to HLMP-1 expression;

FIG. 10 is a chart showing that the LMP-1 mediated increase in sGAG production is blocked by noggin;

FIG. 11 is a graph showing the effect of LMP-1 on sGAG in media after day 6 of culture in monolayer.

FIGS. 12A-12D are photomicrographs of immunohistochemical staining for LMP-1 protein in A549 cells;

FIGS. 13A-13F are photomicrographs of immunohistochemical staining of A549 cells 48 hours after infection with AdLMP-1 (upper panels) or Adβgal (lower panels);

FIGS. 14A-14D are photomicrographs of immunohistochemical staining of A549 cells 48 hours after infection with either AMP-1 (upper panels) or Adβgal (lower panels);

FIGS. 15A-15D are photomicrographs of immunohistochemical staining for the leukocyte surface marker CD45 in human buffy coat cells infected with AdLMP-1 (upper panels) or Adβgal (lower panels) excised at 3 days (FIGS. 15A and 15C) or 5 days (FIGS. 15B and 15D) following implantation with a collagen matrix subcutaneously on the chest of an athymic rat;

FIGS. 16A-16D are photomicrographs of immunohistochemical staining for BMP-4 in human buffy coat cells infected with AdLMP-1 (upper panels) or Adβgal (lower panels) excised at 3 days (FIGS. 16A and 16C) or 5 days (FIGS. 16B and 16D) following implantation with a collagen matrix subcutaneously on the chest of an athymic rat;

FIGS. 17A-17D are photomicrographs of immunohistochemical staining for BMP-7 in human buffy coat cells infected with AdLMP-1 (upper panels) or Adβgal (lower panels) excised at 3 days (FIGS. 17A and 17C) or 5 days (FIGS. 17B and 17D) following implantation with a collagen matrix subcutaneously on the chest of an athymic rat;

FIG. 18 is a high power photomicrograph of immunohistochemical staining for BMP-7 in human huffy coat cells infected with AdLMP-1 excised at 14 days following implantation with a collagen matrix subcutaneously on the chest of an athymic rat;

FIGS. 19A-19D are photomicrographs of human buffy coat cells infected with AdLMP-1 (upper panels) or Adβgal (lower panels) excised at 1 day (FIGS. 19A and 19C) or 3 days (FIGS. 19B and 19D) following implantation in a collagen matrix subcutaneously on the chest of athymic rat

FIGS. 20A and 20B are high power photomicrographs of human buffy coat cells infected with AdLMP-1 or Adβgal excised at 1 day following implantation in a collagen matrix subcutaneously on the chest of an athymic rat;

FIGS. 21A-21J are photomicrographs of human buffy coat cells infected with AdLMP-1 (upper panels-FIGS. 21A-21E) or Adβgal (lower panels-FIGS. 21F-21J) excised at various time points following implantation with a collagen matrix subcutaneously on the chest of an athymic rat;

FIGS. 22A-22C are high power photomicrographs of human buffy coat cells infected with AdLMP-1 excised at various time points following implantation with a collagen matrix subcutaneously on the chest of an athymic rat;

FIG. 23 is a chart showing the effect of the dosage of type 5 AdLMP-1 on the total LMP-1 mRNA levels measured in rabbit disc cells transfected in vivo;

FIG. 24 is a chart showing the effect of the dosage of type 5 AdLMP-1 on the BMP-2 and BMP-7 mRNA levels measured in rabbit disc cells transfected in vivo;

FIG. 25 is a chart showing the effect of the dosage of type 5 AdLMP-1 on the aggrecan mRNA levels measured in rabbit disc cells transfected in vivo;

FIG. 26 is a chart showing the LMP-1 mRNA levels measured in human nucleus pulposus (NP) and annulus fibrosus (AF) cells transfected ex vivo using a type 5/F35 AdLMP-1 adenovirus compared to cells not treated with an adenovirus (NT) and cells treated with a control adenovirus (AdGFP);

FIG. 27 is a chart showing the BMP-2 mRNA levels measured in human nucleus pulposus (NP) cells transfected ex vivo using a type 5/F35 AdLMP-1, adenovirus compared to cells not treated with an adenovirus (NT) and cells treated with a control adenovirus (AdGFP);

FIG. 28 is a chart showing the BMP-2 mRNA levels measured in human annulus fibrosus (AF) cells transfected ex vivo using a type 5/F35 AdLMP-1 adenovirus compared to cells not treated with an adenovirus (NT) and cells treated with a control adenovirus (AdGFP);

FIG. 29 is a chart showing proteoglycan levels measured in human nucleus pulposus (NP) cells transfected ex vivo using a type 5/F35 AdLMP-1 adenovirus compared to cells not treated with an adenovirus (NT) and cells treated with a control adenovirus (AdGFP); and

FIG. 30 is a chart showing proteoglycan levels measured in human annulus fibrosus (AF) cells transfected ex vivo using a type 51F35 AdLMP-1 adenovirus compared to cells not treated with an adenovirus (NT) and cells treated with a control adenovirus (AdGFP).

### DETAILED DESCRIPTION OF THE PREFERRED EMBODIMENTS

LMP-1 is a novel LIM domain protein associated with early osteoblast differentiation. LMP-1 transcripts are first detectable in mesenchymal cells adjacent to the hypertrophic cartilage cells in developing embryonic long bones just before osteoblasts appear at the center of the cartilage anlage. See Boden, et al., “LMP-1, A LIM-Domain Protein, Mediates BMP-6 Effects on Bone Formation”, Endocrinology, 139, 5125-5134 (1998). The LMP-1 protein is a member of the heterogeneous family of LIM domain proteins, many of which are involved with growth and differentiation in a variety of cell types. However, the precise mechanisms of action of LIM-domain proteins remain poorly understood. See Kong, et al., “Muscle LIM Protein Promotes Myogenesis by Enhancing the Activity of MyoD.”, Mol. Cell. Biol., 17, 4750-4760 (1997); Sadler, et al., “Zyxin and cCRP: Two Interactive LIM Domain Proteins Associated with the Cytoskeleton”, J. Cell Biol., 119, 1573-1587 (1992); Salgia, et al., “Molecular Cloning of Human Paxillin, a Focal Adhesion Protein Phosphorylated by P210(BCCR/ABL)”, J. Biol. Chem., 270, 5039-5047 (1995); and Way, et al., “Mec-3, A Homeobox-Containing Gene that Specifies the Differentiation of the Touch Receptor Neurons in C. Elegans”, Cell, 54, 5-16 (1988).

Although LMP-1 is a LIM domain protein, it has recently been shown that the LIM domains themselves are not necessary for osteoblast differentiation. See Liu, et al., “Overexpressed LIM Mineralization Proteins do not Require LIM Domains to Induce Bone”, J. Bone Min. Res., 17, 406-414 (2002). LMP-1 is thought to be a potent intracellular signalling molecule that is capable, at very low doses, of inducing osteoblast differentiation in vitro and de novo bone formation in vivo—yet its mechanism of action remains unknown. Boden, et al., Endocrinology, 139, 5125-5134 (1998), supra.

Four important results have emerged from this series of experiments concerning the mechanism of action of LMP-1. There is now compelling evidence from two separate experimental systems that LMP-1 induces the expression of several BMPs. The evidence is most compelling for BMP-4 and BMP-7 which can be detected as early as 48 hours after insertion of the LMP-1 cDNA in vitro and 72 hours in vivo. In vivo studies showed that most of the implanted buffy coat cells expressing LMP-I survived for less than a week in vivo, but there was indirect evidence of an influx of host cells that differentiated into bone forming cells. Lastly, LMP-1 appears to induce membranous bone formation without a clear cartilage interphase, which is common with many of the BMPs.

In the present study, it has also been shown that cells treated with AdLMP-1 produced LMP-1, BMP-2, and to lesser extent BMP-6 and TGF-β1 protein in vitro. Additionally, BMP-4 and BMP-7 remain two strong candidates for secreted osteoinductive factors induced by IMP-1. We have performed preliminary antisense oligonucleotide experiments which suggest that BMP-4 and BMP-7 were necessary for the osteoinductive effects of LMP-1 to transfer to other cells (unpublished data), but these experiments did not demonstrate whether LMP-1 induced the synthesis of these BMPs.

The A549 experiments described below show that the BMPs were not induced by the adenovirus itself nor were the BMPs expressed in untreated the cells. The A549 experiments also show that two proteins not related to osteoblast differentiation (i.e., type II collagen and MyoD) were not induced by LMP-1.

A549 lung carcinoma cells were chosen rather than osteoblasts because the A549 cells had no basal expression of BMPs. The use of osteoblasts in our experiments we would not have permitted as direct a link between LMP expression and BMP induction to be made. In osteoblasts, any non-specific initiation of osteoblast differentiation would ultimately result in BMP expression and the link to LMP expression would have been less clear. Finally, the in vivo experiments in human buffy coat cells confirmed these observations in cells and in an environment in which bone was actually forming to insure that the observations were true in a physiologic bone formation setting.

The authors recognize that there may be other proteins induced by LMP-1 that include other BMPs or possibly helper proteins that facilitate the action/activity of very small amounts of BMPs as seen in physiologic bone healing situations. This phenomenon would not be surprising given the high potency of small doses of LMP-1 and the difficulty observing its induction of individual BMP proteins by less sensitive techniques such as Western blotting.

The use of buffy coat cells from ordinary venous blood for ex vivo gene therapy is a relatively new concept. See Viggeswarapu, et al., “Adenoviral Delivery of LIM Mineralization Protein-1 Induces New-Bone Formation in vitro and in vivo”, J. Bone Joint Surg. Am., 83-A, 364-376 (2001). One relevant question raised has been how long the buffy coat cells transfected with LMP-1 cDNA survive in vivo and enhance the synthesis, secretion and activity of BMPs. To attempt to answer this question, the CD-45 antigen, which is well-known as a marker of white blood cells, was examined in the present study. See Kurtin, et al., “Leukocyte Common AntigenA Diagnostic Discriminant Between Hematopoietic and Nonhematopoietic Neoplasms in Paraffin Sections using Monoclonal Antibodies: Correlation with Immunologic Studies and Ultrastructural Localization”, Hum. Pathol., 16, 353-365 (1985); and Pulido, et al., “Comparative Biochemical and Tissue Distribution Study of Four Distinct CD45 Antigen Specificities”, J. Immunol., 140, 3851-3857 (1988). The number of cells specifically reacting with the anti-CD-45 primary antibody decreased progressively and as minimal by 10 days following implantation. The loss of anti-CD-45 staining, the dropout of cells in the center of the implant by seven days, and the centripetal pattern of bone formation all suggested that the transplanted cells, including those expressing the LMP-1 cDNA, may not survive long. This observation suggests, but does not confirm, the notion that LMP-expressing cells may only participate indirectly in the bone formation process through induction of secreted factors that subsequently recruit host progenitor cells and modulate their differentiation into mature osteoblasts. LMP-1 seems to start a cascade of events, including the secretion of several osteoinductive proteins (BMPs), and therefore we believe that the expression of LMP-1 does not need to occur in very many cells or need to persist for very long in vivo.

These studies demonstrated the histologic healing sequence of bone induced by ex vivo gene transfer of LMP-1 cDNA to peripheral blood buffy coat cells implanted in an ectopic location. This work has begun to answer some of the questions as to the mechanism of bone formation with LMP-1 at the macroscopic level. A better understanding of the mechanism of action of LMP-1 will facilitate its translation to the clinical setting and improve the understanding of intracellular signalling pathways involved in LMP action.

The present invention relates to the transfection of non-osseous cells with nucleic acids encoding LIM mineralization proteins. The present inventors have discovered that transfection of non-osseous cells such as intervertebral disc cells with nucleic acids encoding LIM mineralization proteins can result in the increased synthesis of proteoglycan, collagen and other intervertebral disc components and tissue. The present invention also provides a method for. treating intervertebral disc disease associated with the loss of proteoglycan, collagen, or other intervertebral disc components.

It is to be understood that both the foregoing general description and the following detailed description are exemplary and explanatory and are intended to provide further explanation of the invention as claimed.

### ABBREVIATIONS AND DEFINITIONS

BMP Bone Morphogenetic Protein
HLMP-1 Human LMP-1, also designated as Human LIM Protein or HLMP
HLMP-1s Human LMP-1 Short (truncated) protein
HLMPU Human LIM Protein Unique Region
LMP LIM mineralization protein
MEM Minimal essential medium
Trm Triamcinolone
βGlyP Beta-glycerolphosphate
RACE Rapid Amplification of cDNA Ends
RLMP Rat LIM mineralization protein, also designated as RLMP-1
RLMPU Rat LIM Protein Unique Region
RNAsin RNase inhibitor
ROB Rat Osteoblast
10-4 Clone containing cDNA sequence for RLMP (SEQ ID NO: 2)
UTR Untranslated Region
HLMP-2 Human LMP Splice Variant 2
HLMP-3 Human LMP Splice Variant 3
MOI multiplicity of infection
sGAG sulfated glycosaminoglycan
AdHLMP-1 Recombinant Type 5 Adenovirus comprising nucleotide sequence encoding HLMP-1

A LIM gene (10-4/RLMP) has been isolated from stimulated rat calvarial osteoblast cultures (SEQ. ID NO: 1, SEQ. ID NO: 2). See U.S. Pat. No. 6,300,127. This gene has been cloned, sequenced and assayed for its ability to enhance the efficacy of bone mineralization in vitro. The protein RLMP has been found to affect the mineralization of bone matrix as well as the differentiation of cells into the osteoblast lineage. Unlike other known cytokines (e.g., BMPs), RLMP is not a secreted protein, but is instead an intracellular signaling molecule. This feature has the advantage of providing intracellular signaling amplification as well as easier assessment of transfected cells. It is also suitable for more efficient and specific in vivo applications. Suitable clinical applications include enhancement of bone repair in fractures, bone defects, bone grafting, and normal homeostasis in patients presenting with osteoporosis.

The amino acid sequence of a corresponding human protein, named human LMP-1 (“HLMP1”), has also been cloned, sequenced and deduced. See U.S. Pat. No. 6,300,127. The human protein has been found to demonstrate enhanced efficacy of bone mineralization in vitro and in vivo.

Additionally, a truncated (short) version of HLMP-1, termed HLMP-1s, has been characterized. See U.S. Pat. No. 6,300,127. This short version resulted from a point mutation in one source of a cDNA clone, providing a stop codon which truncates the protein. HLMP-1s has been found to be fully functional when expressed in cell culture and in vivo.

Using PCR analysis of human heart cDNA library, two alternative splice variants (referred to as HLMP-2 and HLMP-3) have been identified that differ from HLMP-1 in a region between base pairs 325 and 444 in the nucleotide sequence encoding HLMP-1. See U.S. patent application Ser. No. 09/959,578, filed Apr. 28, 2000, pending. The HLMP-2 sequence has a 119 base pair deletion and an insertion of 17 base pairs in this region. Compared to HLMP-1, the nucleotide sequence encoding HLMP-3 has no deletions, but it does have the same 17 base pairs as HLMP-2, which are inserted at position 444 in the HLMP-1 sequence.

LMP is a pluripotent molecule, which regulates or influences a number of biological processes. The different splice variants of LMP are expected to have different biological functions in mammals. They may play a role in the growth, differentiation, and/or regeneration of various tissues. For example, some form of LMP is expressed not only in bone, but also in muscle, tendons, ligaments, spinal cord, peripheral nerves, and cartilage.

According to one aspect, the present invention relates to a method of stimulating proteoglycan and/or collagen synthesis in a mammalian cell by providing an isolated nucleic acid comprising a nucleotide sequence encoding LIM mineralization protein operably linked to a promoter; transfecting said isolated nucleic acid sequence into a mammalian cell capable of producing proteoglycan; and expressing said nucleotide sequence encoding LIM mineralization protein, whereby proteoglycan synthesis is stimulated. The mammalian cell may be a non-osseous cell, such as an intervertebral disc cell, a cell of the annulus fibrosus, or a cell of the nucleus pulposus. Transfection may occur either ex vivo or in vivo by direct injection of virus or naked DNA, such as, for example, a plasmid. In certain embodiments, the virus is a recombinant adenovirus, preferably AdHLMP-1.

Another embodiment of the invention comprises a non-osseous mammalian cell comprising an isolated nucleic acid sequence encoding a LIM mineralization protein. The non-osseous mammalian cell may be a stem cell (e.g., a pluripotential stem cell or a mesenchymal stem cell) or an intervertebral disc cell, preferably a cell of the nucleus pulposus or a cell of the annulus fibrosus.

In a different aspect, the invention is directed to a method of expressing an isolated nucleotide sequence encoding LIM mineralization protein in a non-osseous mammalian cell, comprising providing an isolated nucleic acid comprising a nucleotide sequence encoding LIM mineralization protein operably linked to a promoter; transfecting said isolated nucleic acid sequence into a non-osseous mammalian cell; and expressing said nucleotide sequence encoding LIM mineralization protein. The non-osseous mammalian cell may be a stem cell or an intervertebral disc cell (e.g., a cell of the nucleus pulposus or annulus fibrosus). Transfection may occur either ex vivo or in vivo by direct injection of virus or naked DNA, such as, for example, a plasmid. The virus can be a recombinant adenovirus, preferably AdHLMP-1.

In yet another embodiment, the invention is directed to a method of treating intervertebral disc disease by reversing, retarding, or slowing disc degeneration, comprising providing an isolated nucleic acid comprising a nucleotide sequence encoding LIM mineralization protein operably linked to a promoter; transfecting said isolated nucleic acid sequence into a mammalian cell capable of producing proteoglycan; and stimulating proteoglycan synthesis in said cell by expressing said nucleotide sequence encoding LIM mineralization protein, whereby disc degeneration is reversed, halted or slowed. The disc disease may involve lower back pain, disc herniation, or spinal stenosis. The mammalian cell may be a non-osseous cell, such as a stem cell or an intervertebral disc cell (e.g., a cell of the annulus fibrosus, or a cell of the nucleus pulposus).

Transfection may occur either ex vivo or in vivo by direct injection of virus or naked DNA, such as, for example, a plasmid. In certain embodiments, the virus is a recombinant adenovirus, preferably AdHLMP-1.

The present invention relates to novel mammalian LIM proteins, herein designated LIM mineralization proteins, or LMPs. The invention relates more particularly to human LMP, known as HLMP or HLMP-1, or alternative splice variants of human LMP, which are known as HLMP-2 or HLMP-3. The Applicants have discovered that these proteins enhance bone mineralization in mammalian cells grown in vitro. When produced in mammals, LMP also induces bone formation in vivo.

Ex vivo transfection of bone marrow cells, osteogenic precursor cells, peripheral blood cells and stem cells (e.g., pluripotential stem cells or mesenchymal stem coils) with nucleic acid that encodes a LIM mineralization protein (e.g., LMP or HLMP), followed by reimplantation of the transfected cells in the donor, is suitable for treating a variety of bone-related disorders or injuries. For example, one can use this method to: augment long bone fracture repair; generate bone in segmental defects; provide a bone graft, substitute for fractures; facilitate tumor reconstruction or spine fusion; and provide a local treatment (by injection) for weak or osteoporotic bone, such as in osteoporosis of the hip, vertebrae, or wrist. Transfection with LMP or HLMP-encoding nucleic acid is also useful in: the percutaneous injection of transfected marrow cells to accelerate the repair of fractured long bones; treatment of delayed union or non-unions of long bone fractures or pseudoarthrosis of spine fusions; and for inducing new bone formation in avascular necrosis of the hip or knee.

In addition to ex vivo methods of gene therapy, transfection of a recombinant DNA vector comprising a nucleic acid sequence that encodes LMP or HLMP can be accomplished in vivo. When a DNA fragment that encodes UP or HLMP is inserted into an appropriate viral vector, for example, an adenovirus vector, the viral construct can be injected directly into a body site were endochondral bone formation is desired. By using a direct, percutaneous injection to introduce the LMP or HLMP sequence stimulation of bone formation can be accomplished without the need for surgical intervention either to obtain bone marrow cells (to transfect ex vivo) or to reimplant them into the patient at the site where new bone is required. Alden, et al., Neurosurgical Focus (1998), have demonstrated the utility of a direct injection method of gene therapy using a cDNA that encodes BMP-2, which was cloned into an adenovirus vector.

It is also possible to carry out in vivo gene therapy by directly injecting into an appropriate body site, a naked, that is, unencapsulated, recombinant plasmid comprising a nucleic acid sequence that encodes HLMP. In this embodiment of the invention, transfection occurs when the naked plasmid DNA is taken up, or internalized, by the appropriate target cells, which have been described. As in the case of in vivo gene therapy using a viral construct, direct injection of naked plasmid DNA offers the advantage that little or no surgical intervention is required. Direct gene therapy, using naked plasmid DNA that encodes the endothelial cell mitogen VEGF (vascular endothelial growth factor), has been successfully demonstrated in human patients. Baumgartner, et al., Circulation, 97, 12, 1114-1123 (1998).

For intervertebral disc applications, ex vivo transfection may be accomplished by harvesting cells from an intervertebral disc, transfecting the cells with nucleic acid encoding LMP in vitro, followed by introduction of the cells into an intervertebral disc. The cells may be harvested from or introduced back into the intervertebral disc using any means known to those of skill in the art, such as, for example, any surgical techniques appropriate for use on the spine. In one embodiment, the cells are introduced into the intervertebral disc by injection.

Also according to the invention, stem cells (e.g., pluripotential stem cells or mesenchymal stem cells) can be transfected with nucleic acid encoding a LIM Mineralization Protein ex vivo and introduced into the intervertebral disc (e.g., by injection).

The cells transfected ex vivo can also be combined with a carrier to form an intervertebral disc implant. The carrier comprising the transfected cells can then be implanted into the intervertebral disc of a subject. Suitable carrier materials are disclosed in Helm, et al. “Bone Graft Substitutes for the Promotion of Spinal Arthrodesis”, Neurosurg Focus, 10 (4) (2001). The carrier preferably comprises a biocompatible porous matrix such as a demineralized bone matrix (DBM), a biocompatible synthetic polymer matrix or a protein matrix. Suitable proteins include extracellular matrix proteins such as collagen. The cells transfected with the LMP ex vivo can be incorporated into the carrier (i.e., into the pores of the porous matrix) prior to implantation.

Similarly, for intervertebral disc applications where the cells are transfected in vivo, the DNA may be introduced into the intevertebral disc using any suitable method known to those of skill in the art. In one embodiment, the nucleic acid is directly injected into the intervertebral space.

By using an adenovirus vector to deliver LMP into osteogenic cells, transient expression of LMP is achieved. This occurs because adenovirus does not incorporate into the genome of target cells that are transfected. Transient expression of LMP, that is, expression that occurs during the lifetime of the transfected target cells, is sufficient to achieve the objects of the invention. Stable expression of LMP, however, can occur when a vector that incorporates into the genome of the target cell is used as a delivery vehicle. Retrovirus-based vectors, for example, are suitable for this purpose.

Stable expression of LMP is particularly useful for treating various systemic bone-related disorders, such as osteoporosis and osteogenesis imperfecta. For this embodiment of the invention, in addition to using a vector that integrates into the genome of the target cell to deliver an LMP-encoding nucleotide sequence into target cells, LAP expression can be placed under the control of a regulatable promoter. For example, a promoter that is turned on by exposure to an exogenous inducing agent, such as tetracycline, is suitable.

Using this approach, one can stimulate formation of new bone on a systemic basis by administering an effective amount of the exogenous inducing agent. Once a sufficient quantity of bone mass is achieved, administration of the exogenous inducing agent can be discontinued. This process may be repeated as needed to replace bone mass lost, for example, as a consequence of osteoporosis. Antibodies specific for HLMP are particularly suitable for use in methods for assaying the osteoinductive, that is, bone-forming, potential of patient cells. In this way one can identify patients at risk for slow or poor healing of bone repair. Also, HLMP-specific antibodies are suitable for use in marker assays to identify risk factors in bone degenerative diseases, such as, for example, osteoporosis.

Following well known and conventional methods, the genes of the present invention are prepared by ligation of nucleic acid segments that encode LMP to other nucleic acid sequences, such as cloning and/or expression vectors. Methods needed to construct and analyze these recombinant vectors, for example, restriction endonuclease digests, cloning protocols, mutagenesis, organic synthesis of oligonucleotides and DNA sequencing, have been described. For DNA sequencing DNA, the dieoxyterminator method is the preferred.

Many treatises on recombinant DNA methods have been published, including Sambrook, et al., Molecular Cloning: A Laboratory Manual, 2^{nd} edition, Cold Spring Harbor Press (1988); Davis, et al., Basic-Methods in Molecular Biology, Elsevier (1986), and Ausubel, et al. Current Protocols in Molecular Biology, Wiley Interscience (1988). These reference manuals are specifically incorporated by reference herein.

Primer-directed amplification of DNA or cDNA is a common step in the expression of the genes of this invention. It is typically performed by the polymerase chain reaction (PCR). PCR is described in U.S. Pat. No. 4,800,159 to Mullis, et al. and other published sources. The basic principle of PCR is the exponential replication of a DNA sequence by successive cycles of primer extension. The extension products of one primer, when hybridized to another primer, becomes a template for the synthesis of another nucleic acid molecule. The primer-template complexes act as substrate for DNA polymerase, which in performing its replication function, extends the primers. The conventional enzyme for PCR applications is the thermostable DNA polymerase isolated from Thermus aquaticus, or Taq DNA polymerase.

Numerous variations of the basic PCR method exist, and a particular procedure of choice in any given step needed to construct the recombinant vectors of this invention is readily performed by a skilled artisan. For example, to measure cellular expression of 10-4/RLMP, RNA is extracted and reverse transcribed under standard and well known procedures. The resulting cDNA is then analyzed for the appropriate mRNA sequence by PCR.

The gene encoding the LIM mineralization protein is expressed in an expression vector in a recombinant expression system. Of course, the constructed sequence need not be the same as the original, or its complimentary sequence, but instead may be any sequence determined by the degeneracy of the DNA code that nonetheless expresses an LMP having bone forming, activity. Conservative amino acid substitutions, or other modifications, such as the occurrence of an amino-terminal methionine residue, may also be employed.

A ribosome binding site active in the host expression system of choice is ligated to the 5′ end of the chimeric LMP coding sequence, forming a synthetic gene. The synthetic gene can be inserted into any one of a large variety of vectors for expression by ligating to an appropriately linearized plasmid. A regulatable promoter, for example, the E. coli lac promoter, is also suitable for the expression of the chimeric coding sequences. Other suitable regulatable promoters include trp, tac, recA, T7 and lambda promoters.

DNA encoding LMP is transfected into recipient cells by one of several standard published procedures, for example, calcium phosphate precipitation, DEAE-Dextran, electroporation or protoplast fusion, to form stable transformants. Calcium phosphate precipitation is preferred, particularly when performed as follows.

DNAs are coprecipitated with calcium phosphate according to the method of Graham, et al., Virology, 52, 456 (1973), before transfer into cells. An aliquot of 40-50 μg of DNA, with salmon sperm or calf thymus DNA as a carrier, is used for 0.5×106 cells plated on a 100 mm dish. The DNA is mixed with 0.5 ml of 2× Hepes solution (280 mM NaCl, 50 mM Hepes and 1.5 mM Na₂HPO₄, pH 7.0), to which an equal volume of 2×CaCl₂ (250 mM CaCl₂ and 10 mM Hepes, pH 7.0) is added. A white granular precipitate, appearing after 30-40 minutes, is evenly distributed dropwise on the cells, which are allowed to incubate for 4-16 hours at 37° C. The medium is removed and the cells shocked with 15% glycerol in PBS for 3 minutes. After removing the glycerol, the cells are fed with Dulbecco's Minimal Essential Medium (DMEM) containing 10% fetal bovine serum.

DNA can also be transfected using: the DEAE-Dextran methods of Kimura, et al., Virology, 49:394 (1972) and Sompayrac, et al., Proc. Natl. Acad. Sci. USA, 78, 7575 (1981); the electroporation method of Potter, Proc. Natl. Acad. Sci. USA, 81, 7161 (1984); and the protoplast fusion method of Sandri-Goddin, et al., Molec. Cell. Biol., 1, 743 (1981).

Phosphoramidite chemistry in solid phase is the preferred method for the organic synthesis of oligodeoxynucleotides and polydeoxynucleotides. In addition, many other organic synthesis methods are available. Those methods are readily adapted by those skilled in the art to the particular sequences of the invention.

The present invention also includes nucleic acid molecules that hybridize under standard conditions to any of the nucleic acid sequences encoding the LIM mineralization proteins of the invention. “Standard hybridization conditions” will vary with the size of the probe, the background and the concentration of the nucleic acid reagents, as well as the type of hybridization, for example, in situ, Southern blot, or hybrization of DNA-RNA hybrids (Northern blot). The determination of “standard hybridization conditions” is within the level of skill in the art. For example, see U.S. Pat. No. 5,580,775 to Fremeau, et al., herein incorporated by reference for this purpose. See also, Southern, J. Mol. Biol., 98:503 (1975), Alwine, et al., Meth. Enzymol., 68:220 (1979), and Sambrook, et al., Molecular Cloning: A Laboratory Manual, 2^{nd} edition, Cold Spring Harbor Press, 7.19-7.50 (1989).

One preferred set of standard hybrization conditions involves a blot that is prehybridized at 42° C. for 2 hours in 50% formamide, 5×SSPE (150 nM NaCl, 10 mM Na H₂PO₄ [pH 7.4], 1 mM EDTA [pH 8.0]) 15× Denhardt's solution (20 mg Ficoll, 20 mg polyvinylpyrrolidone and 20 mg BSA per 100 ml water), 10% dextran sulphate, 1% SDS and 100 μg/ml salmon sperm DNA. A ³²P-labeled cDNA probe is added, and hybridization is continued for 14 hours. Afterward, the blot is washed twice with 2×SSPE, 0.1% SDS for 20 minutes at 22° C., followed by a 1 hour wash at 65° C. in 0.1×SSPE, 0.1% SDS. The blot is then dried and exposed to x-ray film for 5 days in the presence of an intensifying screen.

Under “highly stringent conditions,” a probe will hybridize to its target sequence if those two sequences are substantially identical. As in the case of standard hybridization conditions, one of skill in the art can, given the level of skill in the art and the nature of the particular experiment, determine the conditions under which only substantially identical sequences will hybridize.

According to one aspect of the present invention, an isolated nucleic acid molecule comprising a nucleic acid sequence encoding a LIM mineralization protein is provided. The nucleic acid molecule according to the invention can be a molecule which hybridizes under standard conditions to a nucleic acid molecule complementary to the full length of SEQ. ID NO: 25 and/or which hybridizes under highly stringent conditions to a nucleic acid molecule complementary to the full length of SEQ. ID NO: 26. More specifically, the isolated nucleic acid molecule according to the invention can encode HLMP-1, HLMP-1s, RLMP, HLMP-2, or HLMP-3.

Another aspect of the invention includes the proteins encoded by the nucleic acid sequences. In still another embodiment, the invention relates to the identification of such proteins based on anti-LMP antibodies. In this embodiment, protein samples are prepared for Western blot analysis by lysing cells and separating the proteins by SDS-PAGE. The proteins are transferred to nitrocellulose by electroblotting as described by Ausubel, et al., Current Protocols in Molecular Biology, John Wiley and Sons (1987). After blocking the filter with instant nonfat dry milk (1 gm in 100 ml PBS), anti-LMP antibody is added to the filter and incubated for 1 hour at room temperature. The filter is washed thoroughly with phosphate buffered saline (PBS) and incubated with horseradish peroxidase (HRPO)-antibody conjugate for 1 hour at room temperature. The filter is again washed thoroughly with PBS and the antigen bands are identified by adding diaminobenzidine (DAB).

Monospecific antibodies are the reagent of choice in the present invention, and are specifically used to analyze patient cells for specific characteristics associated with the expression of LMP. “Monospecific antibody” as used herein is defined as a single antibody species or multiple antibody species with homogenous binding characteristics for LMP. “Homogeneous binding” as used herein refers to the ability of the antibody species to bind to a specific antigen or epitope, such as those associated with LMP, as described above. Monospecific antibodies to LMP are purified from mammalian antisera containing antibodies reactive against LMP or. are prepared as monoclonal-antibodies reactive with LMP using the technique of Kohler, et al., Nature, 256, 49.5-497 (1975). The LMP specific antibodies. are raised by immunizing animals such as, for example, mice, rats, guinea pigs, rabbits, goats, or horses, with an appropriate concentration of LMP either with or without an immune adjuvant.

In this process, pre-immune serum is collected prior to the first immunization. Each animal receives between about 0.1 mg and about 1000 mg of LMP associated with an acceptable immune adjuvant, if desired. Such acceptable adjuvants include, but are not limited to, Freund's complete, Freund's incomplete, alum-precipitate, water in oil emulsion containing Corynebacterium parvum and tRNA adjuvants. The initial immunization consists of LMP in, preferably, Freund's complete adjuvant injected at multiple sites either subcutaneously (SC), intraperitoneally (IP) or both. Each animal is bled at regular intervals, preferably weekly, to determine antibody titer. The animals may or may not receive booster injections following the initial immunization. Those animals receiving booster injections are generally given an equal amount of the antigen in Freund's incomplete adjuvant by the same route. Booster injections are given at about three week intervals until maximal titers are obtained. At about 7 days after each booster immunization or about weekly after a single immunization, the animals are bled, the serum collected, and aliquots are stored at about −20° C.

Monoclonal antibodies (mAb) reactive with LMP are prepared by immunizing inbred mice, preferably Balb/c mice, with LAP. The mice are immunized by the IP or SC route with about 0.1 mg to about 10 mg, preferably about 1 mg, of LMP in about 0.5 ml buffer or saline incorporated in an equal volume of an acceptable adjuvant, as discussed above. Freund's complete adjuvant is preferred. The mice receive an initial immunization on day 0 and are rested for about 3-30 weeks. Immunized mice are given one or more booster immunizations of about 0.1 to about 10 mg of LMP in a buffer solution such as phosphate buffered saline by the intravenous (IV) route. Lymphocytes from antibody-positive mice, preferably splenic lymphocytes, are obtained by removing the spleens from immunized mice by standard procedures known in the art. Hybridoma cells are produced by mixing the splenic lymphocytes with an appropriate fusion partner, preferably myeloma cells, under conditions which will allow the formation of stable hybridomas. Fusion partners may include, but are not limited to: mouse myelomas P3/NS1/Ag 4-1; MPC-11; S-194 and Sp 2/0, with Sp 2/0 being preferred. The antibody producing cells and myeloma cells are fused in polyethylene glycol, about 1,000 mol. wt., at concentrations from about 30% to about 50%. Fused hybridoma cells are selected by growth in hypoxanthine, thymidine and aminopterin in supplemented Dulbecco's Modified Eagles Medium (DMEM) by procedures known in the art. Supernatant fluids are collected from growth positive wells taking place about days 14, 18, and 21, and are screened for antibody production by an immunoassay such as solid phase immunoradioassay (SPIRA) using LMP as the antigen. The culture fluids are also tested in the Ouchterlony precipitation assay to determine the isotype of the mAb. Hybridoma cells from antibody positive wells are cloned by a technique such as the soft agar technique of MacPherson, “Soft Agar Techniques: Tissue Culture Methods and Applications”, Kruse and Paterson (eds), Academic Press (1973). See, also, Harlow, et al., Antibodies: A Laboratory Manual, Cold Spring Laboratory (1988).

Monoclonal antibodies may also be produced in vivo by injection of pristane-primed Balb/c mice, approximately 0.5 ml per mouse, with about 2×10⁶ to about 6×10⁶ hybridoma cells about 4 days after priming. Ascites fluid is collected at approximately 8-12 days after cell transfer and the monoclonal antibodies are purified by techniques known in the art.

In vitro production in anti-LMP mAb is carried out by growing the hydridoma cell line in DMEM containing about 2% fetal calf serum to obtain sufficient quantities of the specific mAb. The mAb are purified by techniques known in the art.

Antibody titers of ascites or hybridoma culture fluids are determined by various serological or immunological assays, which include, but are not limited to, precipitation, passive agglutination, enzyme-linked immunosorbent antibody (ELISA) technique and radioimmunoassay (RIA) techniques. Similar assays are used to detect the presence of the LMP in body fluids or tissue and cell extracts.

It is readily apparent to those skilled in the art that the above described methods for producing monospecific antibodies may be utilized to produce antibodies specific for polypeptide fragments of LMP, full-length nascent LMP polypeptide, or variants or alleles thereof.

In another embodiment, the invention is directed to alternative splice variants of HLMP-1. PCR analysis of human heart cDNA revealed mRNA for two HLMP alternative splice variants, named HLMP-2 and HLMP-3, that differ from HLMP-1 in a region between base pairs 325 and 444 in the HLMP-1 sequence. The HLMP-2 sequence has a 119 base pair deletion and an insertion of 17 base pairs in this region. These changes preserve-the reading frame, resulting in a 423 amino acid: protein, which compared to HLMP-1, has a net loss of 34 amino acids (40 amino acids deleted plus 6 inserted amino acids). HLMP-2 contains the c-terminal LIM domains that are present in HLMP1.

Compared to HLMP-1, HLMP-3 has no deletions, but it does have the same 17 base pair insertion at position 444. This insertion shifts the reading frame, causing a stop codon at base pairs 459-461. As a result, HLMP-3 encodes a protein of 153 amino acids. This protein lacks the c-terminal LIM domains that are present in HLMP-1 and HLMP-2. The predicted size of the proteins encoded by HLMP-2 and HLMP-3 was confirmed by western blot analysis.

PCR analysis of the tissue distribution of the three splice variants revealed that they are differentially expressed, with specific isoforms predominating in different tissues. HLMP-1 is apparently the predominant form expressed in leukocytes, spleen, lung, placenta, and fetal liver. HLMP-2 appears to be the predominant isoform in skeletal muscle, bone marrow, and heart tissue. HLMP-3, however, was not the predominant isoform in any tissue examined.

Over-expression of HLMP-3 in secondary rat osteoblast cultures induced bone nodule formation (287±56) similar to the effect seen for glucicorticoid (272±7) and HLMP-1 (232±200). Since HLMP-3 lacks the C-terminal LIM domains, there regions are not required for osteoinductive activity.

Over-expression of HINT-2, however, did not induce nodule formation (11±3). These data suggest that the amino acids encoded by the deleted 119 base pairs are necessary for osteoinduction. The data also suggest that the distribution of HLMP splice variants may be important for tissue-specific function. Surprisingly, we have shown that HLMP-2 inhibits steroid-induced osteoblast formation; in secondary rat osteoblast cultures. Therefore, HLMP-2 may have therapeutic utility in clinical situations where bone formation is not desirable.

On Jul. 22, 1997, a sample of 10-4/RLMP in a vector designated pCMV2/RLMP (which is vector designated pCMV2/RLMP (which is vector pRc/CMV2 with insert 10-4 clone/RLMP) was deposited with the American Type Culture Collection (ATCC), 12301 Parklawn Drive, Rockville, Md. 20852. The culture accession number for that deposit is 209153. On Mar. 19, 1998, a sample of the vector pHis-A with insert HLPM-1s was deposited at the American Type Culture Collection (“ATCC”). The culture accession number for that deposit is 209698. On Apr. 14, 2000, samples of plasmids pHAhLMP-2 (vector pHisA with cDNA insert derived from human heart muscle cDNA with HLMP-2) and pHAhLMP-3 (vector pHisA with cDNA insert derived from human heart muscle cDNA with HLMP-3) were deposited with the ATCC, 10801 University Blvd., Manassas, Va., 20110-2209, USA, under the conditions of the Budapest treaty. The accession numbers for these deposits are PTA-1698 and PTA-1699, respectively. These deposits, as required by the Budapest Treaty, will be maintained in the ATCC for at least 30 years and will be made available to the public upon the grant of a patent disclosing them. It should be understood that the availability of a deposit does not constitute a license to practice the subject invention in derogation of patent rights granted by government action.

In assessing the nucleic acids, proteins, or antibodies of the invention, enzyme assays, protein purification, and other conventional biochemical methods are employed. DNA and RNA are analyzed by Southern blotting and Northern blotting techniques, respectively. Typically, the samples analyzed are size fractionated by gel electrophoresis. The DNA or RNA in the gels are then transferred to nitrocellulose or nylon membranes. The blots, which are replicas of sample patterns in the gets, were then hybridized with probes. Typically, the probes are radio-labeled, preferably with 32P, although one could label the probes with other signal-generating molecules known to those in the art. Specific bands of interest can then be visualized by detection systems, such as autoradiography.

For purposes of illustrating preferred embodiments of the present invention, the following, non-limiting examples are included. These results demonstrate the feasibility of inducing or enhancing the formation of bone using the LIM mineralization proteins of the invention, and the isolated nucleic acid molecules encoding those proteins.

### EXAMPLE 1

### Calvarial Cell Culture

Rat calvarial cells, also known as rat osteoblasts (“ROB”), were obtained from 20-day pre-parturition rats as previously described. Boden, et al., Endocrinology, 137, 8, 3401-3407 (1996). Primary cultures were grown to confluence (7 days), trypsinized, and passed into 6-well plates (1×10⁵ cells/35 mm well) as first subculture cells. The subculture cells, which were confluent at day 0, were grown for an additional 7 days. Beginning on day 0, media were changed and treatments (Trm and/or BMPS) were applied, under a laminar flow hood, every 3 or 4 days. The standard culture protocol was as follows: days 1-7, MEM, 10% FBS, 50 μg/ml ascorbic acid, ±stimulus; days 8-14, BGJb medium, 10% FBS, 5 mM β-GlyP (as a source of inorganic phosphate to permit mineralization). Endpoint analysis of bone nodule formation and osteocalcin secretion was performed at day 14. The dose of BMP was chosen as 50 ng/ml based on pilot experiments in this system that demonstrated a mid-range effect on the dose-response curve for all BMPs studied.

### EXAMPLE 2

### Antisense Treatment and Cell Culture

To explore the potential functional role of LMP-1 during membranous bone formation, we synthesized an antisense oligonucleotide to block LMP-1 mRNA translation and treated secondary osteoblast cultures that were undergoing differentiation initiated by glucocorticoid. Inhibition of RLMP expression was accomplished with a highly specific antisense oligonucleotide (having no significant homologies to known rat sequences) corresponding to a 25 bp sequence spanning the putative translational start site (SEQ. ID NO: 42). Control cultures either did not receive oligonucleotide or they received sense oligonucleotide. Experiments were performed in the presence (preincubation) and absence of lipofectamine. Briefly, 22 μg of sense or antisense RLMP oligonucleotide was incubated in MEM for 45 minutes at room temperature. Following that incubation, either more MEM or pre-incubated lipofectamine/MEM (7% v/v; incubated 45 minutes at room temperature) was added to achieve an oligonucleotide concentration of 0.2 μM. The resulting mixture was incubated for 15 minutes at room temperature. Oligonucleotide mixtures were then mixed with the appropriate medium, that is, MEM/Ascorbate/±Trm, to achieve a final oligonucleotide concentration of 0.1 μM.

Cells were incubated with the appropriate medium (stimulus) in the presence or absence of the appropriate oligonucleotides. Cultures originally incubated with lipofectamine were re-fed after 4 hours of incubation (37° C.; 5% CO2) with media containing neither lipofectamine nor oligonucleotide. All cultures, especially cultures receiving oligonucleotide, were re-fed every 24 hours to maintain oligonucleotide levels.

LMP-1 antisense oligonucleotide inhibited mineralized nodule formation and osteocalcin secretion in a dose-dependent manner, similar to the effect of BMP-6 oligonucleotide. The LMP-1 antisense block in osteoblast differentiation could not be rescued by addition of exogenous BMP-6, while the BMP-6 antisense oligonucleotide inhibition was reversed with addition of BMP-6. This experiment further confirmed the upstream position of LMP-1 relative to BMP-6 in the osteoblast differentiation pathway. LMP-1 antisense oligonucleotide also inhibited spontaneous osteoblast differentiation in primary rat osteoblast cultures.

### EXAMPLE 3

### Quantitation of Mineralized Bone Nodule Formation

Cultures of ROBs prepared according to Examples 1 and 2 were fixed overnight in 70% ethanol and stained with von Kossa silver stain. A semi-automated computerized video image analysis system was used to quantitate nodule count and nodule area in each well. Boden, et al., Endocrinology, 137, 8, 3401-3407 (1996). These values were then divided to calculate the area per nodule values. This automated process was validated against a manual counting technique and demonstrated a correlation coefficient of 0.92 (p<0.000001). All data are expressed as the mean±standard error of the mean (S.E.M.) calculated from 5 or 6 wells at each condition. Each experiment was confirmed at least twice using cells from different calvarial preparations.

### EXAMPLE A

### P Antitation of Osteocalcin. Secretion

Osteocalcin levels in the culture media were measured using a competitive radioimmunoassay with a monospecific polygonal antibody (Pab) raised in our laboratory against the C-terminal nonapeptide of rat osteocalcin as described in Nanes, et al., Endocrinology, 127:588 (1990). Briefly, 1 μg of nonapeptide was iodinated with 1 mCi ¹²⁵I-Na by the lactoperoxidase method. Tubes containing 200 gl of assay buffer (0.02 M sodium phosphate, 1 mM EDTA, 0.001% thimerosal, 0.025% BSA) received media taken from cell cultures or osteocalcin standards (0-12,000 fmole) at 100 gl/tube in assay buffer. The Pab (1:40,000; 100 μl) was then added, followed by the iodinated peptide (12,000 cpm; 100 μl). Samples tested for non-specific binding were prepared similarly but contained no antibody.

Bound and free PAbs were separated by the addition of 700 μl goat antirabbit IgG, followed by incubation for 18 hours at 4° C. After samples were centrifuged at 1200 rpm for 45 minutes, the supernatants were decanted and the precipitates counted in a gamma counter. Osteocalcin values were reported in fmole/100 μl, which was then converted to pmole/ml medium (3-day production) by dividing those values by 100. Values were expressed as the mean±S.E.M. of triplicate determinations for 5-6 wells for each condition. Each experiment was confirmed at least two times using cells from different calvarial preparations.

### EXAMPLE 5

### Effect of Trm and RLMP on Mineralization In Vitro

There was little apparent effect of either the sense or antisense oligonucleotides on the overall production of bone nodules in the non-stimulated cell culture system. When ROBs were stimulated with Trm, however, the antisense oligonucleotide, to RLMP inhibited mineralization of nodules by >95%.” The addition of exogenous BMP-6 to the oligonucleotide-treated cultures did not rescue the mineralization of RLMP-antisense-treated nodules.

Osteocalcin has long been synonymous with bone mineralization, and osteocalcin levels have been correlated with nodule production and mineralization. The RLMP-antisense oligonucleotide significantly decreases osteocalcin production, but the nodule count in antisense-treated cultures does not change significantly. In this case, the addition of exogenous BMP-6 only rescued the production of osteocalcin in RLMP-antisense-treated cultures by 10-15%. This suggests that the action of RLMP is downstream of, and more specific than, BMP-6.

### EXAMPLE 6

### Harvest and Purification of RNA

Cellular RNA from duplicate wells of ROBs (prepared according to Examples 1 and 2 in 6-well culture dishes) was harvested using 4M guanidine isothiocyanate (GIT) solution to yield statistical triplicates. Briefly, culture supernatant was aspirated from the wells, which were then overlayed with 0.6 ml of GIT solution per duplicate well harvest. After adding the GIT solution, the plates were swirled for 5-10 seconds (being as consistent as possible). Samples were saved at −70° C. for up to 7 days before further processing.

RNA was purified by a slight modification of standard methods according to Sambrook, et al. Molecular Cloning: a Laboratory Manual, Chapter 7.19, 2^{nd} Edition, Cold Spring Harbor Press (1989). Briefly, thawed samples received 60 μl 2.0 M sodium acetate (pH 4.0) 550 μl phenol (water saturated) and 150 μl chloroform:isoamyl alcohol (49:1). After vortexing, the samples were centrifuged (10000×g; 20 minutes; 4° C.), the aqueous phase transferred to a fresh tube, 600 μl isopropanol was added and the RNA precipitated overnight at −20° C.

Following the overnight incubation, the samples were centrifuged (10000×g; 20 minutes) and the supernatant was aspirated gently. The pellets were resuspended in 400 μl DEPC-treated water, extracted once with phenol:chloroform (1:1), extracted with chloroform:isoamyl alcohol (24:1) and precipitated overnight at −20° C. after addition of-40 μl sodium acetate (3.0 M; pH 5.2) and 1.0 ml absolute ethanol. To recover the cellular RNA, the samples were centrifuged (10000×g; 20 min), washed once with 70% ethanol, air dried for 5-10 minutes and resuspended in 20 μl of DEPC-treated water. RNA concentrations were calculated from optical densities that were determined with a spectrophotometer.

### EXAMPLE 7

### Reverse Transcription-Polymerase Chain Reaction

Heated total RNA (5 μg in 10.5 μl total volume DEPC-H₂O at 65° C. for 5 minutes) was added to tubes containing 4 μl 5×MMLV-RT buffer, 2 μl dNTPs, 2 μl dT17 primer (10 pmol/ml), 0.5 μl RNAsin (40 U/ml) and 1 μl MMLV-RT (200 units/μl). The samples were incubated at 37° C. for 1 hour, then at 95° C. for 5 minutes to inactivate the MMLV-RT. The samples were diluted by addition of 80 μl of water.

Reverse-transcribed samples (5 μl) were subjected to polymerase-chain reaction using standard methodologies (50 μl total volume). Briefly, samples were added to tubes containing water and appropriate amounts of PCR buffer, 25 mm MgCl₂, dNTPs, forward and reverse primers for glyceraldehyde 3-phosphate dehydrogenase (GAP, a housekeeping gene) and/or BMP-6, ³²P-dCTP, and Taq polymerase. Unless otherwise: noted, primers were standardized to run consistently at 22 cycles (94° C., 30″; 58° C., 30″; 72° C., 20″).

### EXAMPLE 8

### Quantitation of RT-PCR Products by Polyacrylamide Gel Electrophoresis (PAGE) and PhosphorImager Analysis

RT-PCR products received 5 μl/tube loading dye, were mixed, heated at 65° C. for 10 min and centrifuged. Ten μl of each reaction was subjected to PAGE (12% polyacrylamide:bis; 15 V/well; constant current) under standard conditions. Gels were then incubated in gel preserving buffer (10% v/v glycerol, 7% v/v acetic acid, 40% v/v methanol, 43% deionized water) for 30 minutes, dried (80° C.) in vacuo for 1-2 hours and developed with an electronically-enhanced phosphoresence imaging system for 6-24 hours. Visualized bands were analyzed. Counts per band were plotted graphically.

### EXAMPLE 9

### Differential Display PCR

RNA was extracted from cells stimulated with glucocorticoid (Trm, 1 nM). Heated, DNase-treated total RNA (5 μg in 10.5 μl total volume in DEPC-H₂O at 65° C. for 5 minutes) was reverse transcribed as described in Example 7, but H-T₁₁, M (SEQ. ID. NO: 4) was used as the MMLV-RT primer. The resulting cDNAs were PCR-amplified as described above, but with various commercial primer sets (for example, H-T₁₁G (SEQ. ID NO: 4) and H-AP-10 (SEQ. ID NO: 5); GenHunter Corp, Nashville, Tenn.). Radio-labeled PCR products were fractionated by gel electrophoresis on a DNA sequencing gel. After electrophoresis, the resulting gels were dried, in vacuo and autoradiographs were exposed overnight. Bands representing differentially-expressed cDNAs were excised from the gel and reamplified by PCR using the method of Conner, et al., Proc. Natl. Acad. Sci. USA, 88, 278 (1983). The products of PCR reamplification were cloned into the vector PCR-11 (TA cloning kit; In Vitrogen, Carlsbad, Calif.).

### EXAMPLE 10

### Screening of a UMR 106 Rat Osteosarcoma Cell cDNA Library

A UMR 106 library (2.5×10¹⁰ pfu/ml) was plated at 5×10⁴ pfu/ml onto agar plates (LB bottom agar) and the plates were incubated overnight at 37° C. Filter membranes were overlaid onto plates for two minutes. Once removed, the filters were denatured, rinsed, dried and UV cross-linked. The filters were then incubated in pre-hybridization buffer (2×PIPES [pH 6.5], 5% formamide, 1% SDS and 100 μg/ml denatured salmon sperm DNA) for 2 h at 42° C. A 260 base-pair radio-labeled probe (SEQ. ID NO: 3; ³²P labeled by random priming) was added to the entire hybridization mix/filters, followed by hybridization for 18 hours at 42° C. The membranes were washed once at room temperature (10 min, 1×SSC, 0.1% SDS) and three times at 55° C. (15 min, 0.1×SSC, 0.1% SDS).

After they were washed, the membranes were analyzed by autoradiography as described above. Positive clones were plaque purified. The procedure was repeated with a second filter for four minutes to minimize spurious positives. Plaque-purified clones were rescued as lambda SK(−) phagemids. Cloned cDNAs were sequenced as described below.

### EXAMPLE 11

### Sequencing of Clones

Cloned cDNA inserts were sequenced by standard methods. Ausubel et al., Current Protocols in Molecular Biology, Wiley Interscience (1988). Briefly, appropriate concentrations of termination mixture, template and reaction mixture were subjected to an appropriate cycling protocol (95° C., 30 s; 68° C., 30 s; 72° C., 60 s; ×25). Stop mixture was added to terminate the sequencing reactions. After heating at 92° C. for 3 minutes, the samples were loaded onto a denaturing 6% polyacrylamide sequencing gel (29:1 acrylamide:bisacrylamide). Samples were electrophoresed for about 4 hours at 60 volts, constant current. After electrophoresis, the gels were dried in vacuo and autoradiographed.

The autoradiographs were analyzed manually. The resulting sequences were screened against the databases maintained by the National Center for Biotechnology Information (NM, Bethesda, Md.; hftp://www.ncbi.nlm.nih.gov/) using the BLASTN program set with default parameters. Based on the sequence data, new sequencing primers were prepared and the process was repeated until the entire gene had been sequenced. All sequences were confirmed a minimum of three times in both orientations.

Nucleotide and amino acid sequences were also analyzed using the PCGENE software package (version 16.0). Percent homology values for nucleotide sequences were calculated by the program NALIGN, using the following parameters: weight of non-matching nucleotides, 10; weight of non-matching gaps, 10; maximum number of nucleotides considered, 50; and minimum number of nucleotides considered, 50.

For amino acid sequences, percent homology value were calculated using PALIGN. A value of 10 was selected for both the open gap cost and the unit gap cost.

### EXAMPLE 12

### Cloning of RLMP cDNA

The differential display PCR amplification products described in Example 9 contained a major band of approximately 260 base pairs. This sequence was used to screen a rat osteosarcoma (UMR 106) cDNA library. Positive clones were subjected to nested primer analysis to obtain the primer sequences necessary for amplifying the full length cDNA. (SEQ. ID NOs: 11, 12, 29, 30 and 31). One of those positive clones selected for further study was designated clone 10-4.

Sequence analysis of the full-length cDNA in clone 10-4, determined by nested primer analysis, showed that clone 10-4 contained the original 260 base-pair fragment identified by differential display PCR Clone 10-4 (1696 base pairs; SEQ ID NO: 2) contains an open reading frame of 1371 base pairs encoding a protein having 457 amino acids (SEQ. ID NO: 1). The termination codon, TGA, occurs at nucleotides 1444-1446. The polyadenylation signal at nucleotides 1675-1680, and adjacent poly(A)⁺ tail, was present in the 3′ noncoding region. There were two potential N-glycosylation sites, Asn-Lys-Thr and Asn-Arg-Thr, at amino acid positions 113-116 and 257-259 in SEQ. ID NO: 1, respectively. Two potential cAMP- and cGMP-dependent protein kinase phosphorylation sites, Ser and Thr, were found at amino acid positions 191 and 349, respectively. There were five potential protein kinase C phosphorylation sites, Ser or Thr, at amino acid positions 3, 115, 166, 219, 442. One potential ATP/GTP binding site motif A (P-loop), Gly-Gly-Ser-Asn-Asn-Gly-Lys-Thr, was determined at amino acid positions 272-279.

In addition, two highly conserved putative LIM domains were found at amino acid positions 341-391 and 400-451. The putative LIM domains in this newly identified rat cDNA clone showed considerable homology with the LIM domains of other known LIM proteins. However, the overall homology with other rat LIM proteins was less than 25%. RLMP (also designated 10-4) has 78.5% amino acid homology to the human enigma protein (see U.S. Pat. No. 5,504,192), but only 24.5% and 22.7% amino acid homology to its closest rat homologs, CLP-36 and RIT-18, respectively.

### EXAMPLE 13

### Northern Blot Analysis of RLMP Expression

Thirty μg of total RNA from ROBs, prepared according to Examples 1 and 2, was size fractionated by formaldehyde gel electrophoresis in 1% agarose flatbed gels and osmotically transblotted to nylon membranes. The blot was probed with a 600 base pair EcoRI fragment of full-length 10-4 cDNA labeled with ³²P-dCTP by random priming.

Northern blot analysis showed a 1.7 kb mRNA species that hybridized with the RLMP probe. RLMP mRNA was up-regulated approximately 3.7-fold in ROBs after 24 hours exposure to BMP-6. No up-regulation of RMLP expression was seen in BMP-2 or BMP-4-stimulated ROBs at 24 hours.

### EXAMPLE 14

### Statistical Methods

For each reported nodule/osteocalcin result, data from 5-6 wells from a representative experiment were used to calculate the mean±S.E.M. Graphs may be shown with data normalized to the maximum value for each parameter to allow simultaneous graphing of nodule counts, mineralized areas and osteocalcin.

For each reported RT-PCR, RNase protection assay or Western blot analysis, data from triplicate samples of representative experiments, were used to determine the mean±S.E.M. Graphs may be shown normalized to either day 0 or negative controls and expressed as fold-increase above control values.

Statistical significance was evaluated using a one-way analysis of variance with post-hoc multiple comparison corrections of Bonferroni as appropriate. D. V. Huntsberaer, “The Analysis of Variance”, Elements of Statistical Variance, P. Billingsley (ed.), Allyn & Bacon Inc., Boston, Mass., 298-330 (1977) and SigmaStat, Jandel Scientific, Corte Madera, Calif. Alpha levels for significance were defined as p<0.05.

### EXAMPLE 15

### Detection of Rat LIM Mineralization Protein by Western Blot Analysis

Polyclonal antibodies were prepared according to the methods of England, et al., Biochim. Biophys. Acta, 623, 171 (1980) and Timmer, et al., J. Biol. Chem., 268, 24863 (1993).

HeLa cells were transfected with pCMV2/RLMP. Protein was harvested from the transfected cells according to the method of Hair, et al., Leukemia Research, 20, 1 (1996). Western Blot Analysis of native RLMP was performed as described by Towbin. et al., Proc. Natl. Acad. Sci. USA, 76:4350 (1979).

### EXAMPLE 16

### Synthesis of the Rat LMP-Unique, MPU) Derived-Hunan PCR Product

Based on the sequence of the rat LMP-1 cDNAjorward and reverse PCR primers (SEQ. ID NOS: 15 and 16) were synthesized and a unique 223 base-pair sequence was PCR amplified from the rat LMP-1 cDNA. A similar PCR product was isolated from human MG63 osteosarcoma cell cDNA with the same PCR primers.

RNA was harvested from MG63 osteosarcoma cells grown in T-75 flasks. Culture supernatant was removed by aspiration and the flasks were overlayed with 3.0 ml of GIT solution per duplicate, swirled for 5-10 seconds, and the resulting solution was transferred to 1.5 ml eppendorf tubes (6 tubes with 0.6 ml/tube). RNA was purified by a slight modification of standard methods, for example, see Sambrook, et al., Molecular Cloning: A Laboratory Manual, Chapter 7, page 19, Cold Spring Harbor Laboratory Press (1989) and Boden, et al., Endocrinology, 138, 2820-2828 (1997). Briefly, the 0.6 ml samples received 60 μl 2.0 M sodium acetate (pH 4.0), 550 μl water saturated phenol and 150 μl chloroform:isoamyl alcohol (49:1). After addition of those reagents, the samples were vortexed, centrifuged (10000×g; 20 min; 4 C) and the aqueous phase transferred to a fresh tube. Isopropanol (600 μl) was added and the RNA was precipitated overnight at −20° C. The samples were centrifuged (10000×g; 20 minutes) and the supernatant was aspirated gently. The pellets were resuspended in 400 μl of DEPC-treated water, extracted once with phenol:chloroform (1:1), extracted with chloroform:isoamyl alcohol (24:1) and precipitated overnight at −20° C. in 40 μl sodium acetate (3.0 M; pH 5.2) and. 1.0 ml absolute ethanol. After precipitation, the samples were centrifuged: (10000×g; 20 min), washed once with 70% ethanol, air dried for 5-10 minutes and resuspended in 20 μl of DEPC-treated water. RNA concentrations were derived from optical densities.

Total RNA (5 μg in 10.5 μl total volume in DEPC-H₂O) was heated at 65° C. for 5 minutes, and then added to tubes containing 4 μl 5×MMLV-RT buffer, 2 μl dNTPS, 2 μl dT17 primer (10 pmol/ml), 0.5 μl RNAsin (40 U/ml) and 1 μl MMLV-RT (200 units/μl). The reactions were incubated at 37° C. for 1 hour. Afterward, the MMLV-RT was inactivated by heating at 95° C. for 5 minutes. The samples were diluted by addition of 80 μl water.

Transcribed samples (5 μl) were subjected to polymerase-chain reaction using standard methodologies (50 μl total volume). Boden, et al., Endocrinology, 138, 2820-2828 (1997); Ausubel, et al., “Quantitation of Rare DNAs by the Polymerase Chain Reaction”, Current Protocols in Molecular Biology, Chapter 15.31-1, Wiley & Sons, Trenton, N.J. (1990). Briefly, samples were added to tubes containing water and appropriate amounts of PCR buffer (25 mM MgCl₂, dNTPs, forward and reverse primers (for RLMPU; SEQ. ID NOS: 15 and 16), ³²P-dCTP, and DNA polymerase. Primers were designed to run consistently at 22 cycles for radioactive band detection and 33 cycles for amplification of PCR product for use as a screening probe (94° C., 30 sec, 58° C., 30 sec; 72° C., 20 sec).

Sequencing of the agarose gel-purified MG63 osteosarcoma-derived PCR product gave a sequence more than 95% homologous to the RLMPU PCR product. That sequence is designated JUMP unique region (HLMPU; SEQ. ID NO: 6).

### EXAMPLE 17

### Screening of Reverse-Transcriptase-Derived MG63 cDNA

Screening was performed with PCR using specific primers (SEQ. ID NOS:16 and 17) as described in Example 7. A 717 base-pair MG63 PCR product was agarose gel purified and sequenced with the given primers (SEQ. ID NOs: 12, 15, 16, 17, 18, 27 and 28). Sequences were confirmed a minimum of two times in both directions. The MG63 sequences were aligned against each other and then against the full-length rat LMP cDNA sequence to obtain a partial human LMP cDNA sequence (SEQ. ID NO: 7).

### EXAMPLE 18

### Screening of a Human Heart cDNA Library

Based on Northern blot experiments, it was determined that LMP-1 is expressed at different levels by several different tissues, including human heart muscle. A human heart cDNA library was therefore examined. The library was plated at 5×10⁴ pfu/ml onto agar plates (LB bottom agar) and plates were grown overnight at 37° C. Filter membranes were overlaid onto the plates for two minutes. Afterward, the filters denatured, rinsed, dried, UV cross-linked and incubated in pre-hyridization buffer (2×PIPES [pH 6.5]; 5% formamide, 1% SDS, 100 g/ml denatured salmon sperm DNA) for 2 h at 42° C. A radio-labeled, LMP-unique, 223 base-pair probe (³²P, random primer labeling; SEQ ID NO: 6) was added and hybridized for 18 h at 42° C. Following hybridization, the membranes were washed once at room temperature (10 min, 1×SSC, 0.1% SDS) and three times at 55° C. (15 min, 0.1×SSC, 0.1% SDS). Double-positive plaque-purified heart library clones, identified by autoradiography, were rescued as lambda phagemids according to the manufacturers' protocols (Stratagene, La Jolla, Calif.).

Restriction (digests of positive clones yielded cDNA inserts of varying sizes. Inserts greater, than 600 base-pairs in length were selected for initial screening by sequencing. Those: inserts were sequenced by standard methods as described in Example 11.

One clone, number 7, was also subjected to automated sequence analysis using primers corresponding to SEQ. ID NOS: 11-14, 16 and 27. The sequences obtained by these methods were routinely 97-100% homologous. Clone 7 (Partial Human LMP-1 cDNA from a heart library; SEQ. ID NO: 8) contained a sequence that was more than 87% homologous to the rat LMP cDNA sequence in the translated region.

### EXAMPLE 19

### Determination of Full-Length Human LMP-1 cDNA

Overlapping regions of the MG63 human osteosarcoma cell cDNA sequence and the human heart cDNA clone 7 sequence were used to align those two sequences and derive a complete human cDNA sequence of 1644 base-pairs. NALIGN, a program in the PCGENE software package, was used too align the two sequences. The overlapping regions of the two sequences constituted approximately 360 base-pairs having complete homology except for a single nucleotide substitution at nucleotide 672 in the MG63 cDNA (SEQ. ID NO: 7) with clone 7 having an “A” instead of a “G” at the corresponding nucleotide 516 (SEQ. ID NO: 8).

The two aligned sequences were joined using SEQIN, another subprogram of PCGENE, using the “G” substitution of the MG63 osteosarcoma cDNA clone. The resulting sequence is shown in SEQ. ID NO: 9. Alignment of the novel human-derived sequence with the rat LMP-1 cDNA was accomplished with NALIGN. The full-length human LMP-1 cDNA sequence (SEQ. II) NO: 9) is 87.3% o homologous to the translated portion of rat LMP-1 cDNA sequence.

### EXAMPLE 20

### Determination of Amino Acid Sequence of Human LMP-1

The putative amino acid sequence of human LMP-1 was determined with the PCGENE subprogram TRANSL. The open reading frame in SEQ. ID NO: 9 encodes a protein comprising 457 amino acids (SEQ. ID NO: 10). Using the PCGENE subprogram Palign, the human LMP-1 amino acid sequence was found to be 94.1% homologous to the rat LMP-1 amino acid sequence.

### EXAMPLE 21

### Determination of the 5 Prime Untranslated Region of the Human LAP cDNA

MG63 5′ cDNA was amplified by nested RT-PCR of MG63 total RNA using a 5′ rapid amplification of cDNA ends (TRACE) protocol. This method included first strand cDNA synthesis using a lock-docking oligo (dT) primer with two degenerate nucleotide positions at the 3′ end (Chencllik, et al., CLONTECHniques, X: 5 (1995); Borson, et al., PC Methods Applic., 2, 144 (1993)). Second-strand synthesis is performed according to the method of Gubler, et al., Gene, 2, 263 (1983), with a cocktail of Escherichia coli DNA polymerase 1, RNase H, and E. coli DNA ligase. After creation of blunt ends with T4 DNA polymerase, double-stranded cDNA was ligated to the fragment (5′-CTAATACGACTCACTATAGGGCTCGAGCGGCCGCCCGGGCAGGT-3′) (SEQ. ID NO: 19). Prior to RACE, the adaptor-ligated cDNA was diluted to a concentration suitable for-Marathon RACE-reactions (1:50). Adaptor-ligated double-stranded cDNA was then ready to be specifically cloned.

First-round PCR was performed with the adaptor-specific oligonucleotide, 5′-CCATCCTAATACGACTCACTATAGGGC-3′ (API) (SEQ. ID NO: 20) as sense primer and a Gene Specific Primer (GSP) from the unique region described in Example 16 (HLMPU). The second round of PCR was performed using a nested primers GSPI-HLMPU (antisense/reverse primer) (SEQ. ID NO: 23) and GSP2-HLMPUF (SEQ. ID NO: 24) (see Example 16; sense/forward primer). PCR was performed using a commercial kit (Advantage cDNA PCR core kit; CloneTech Laboratories Inc., Palo Alto, Calif.) that utilizes an antibody-mediated, but otherwise standard, hot-start protocol. PCR conditions for MG63 cDNA included an initial hot-start denaturation (94° C., 60 sec) followed by: 94° C., 30 sec; 60° C., 30 sec; 68° C., 4 min; 30 cycles. The first round PCR product was approximately 750 base-pairs in length whereas the nested PCR product was approximately 230 base-pairs. The first-round PCR product was cloned into linearized pCR 2.1 vector (3.9 Kb). The inserts were sequenced in both directions using M13 Forward and Reverse primers (SEQ. ID NO: 11; SEQ. ID NO: 12).

### EXAMPLE 22

### Determination of Full-length Human LMP-1 cDNA with 5 Prime UTR

Overlapping MG63 human osteosarcoma cell cDNA 5′-UTR sequence (SEQ. ID NO: 21), MG63 717 base-pair sequence (Example 17; SEQ. ID NO: 8) and human heart cDNA clone 7 sequence (Example 18) were aligned to derive a novel human cDNA sequence of 1704 base-pairs (SEQ. ID NO: 22). The alignment-was accomplished with NALIGN, (both PCGENE and Omiga 1.0; Intelligenetics). Over-lapping sequences constituted nearly the entire 717 base-pair region (Example 17) with 100% homology. Joining of the aligned sequences was accomplished with SEQIN.

### EXAMPLE 23

### Construction of LIM Protein Expression Vector

The construction of pHIS-5ATG LMP-1s expression vector was carried out with the sequences described in Examples 17 and 18. The 717 base-pair clone (Example 17; SEQ. ID NO: 7) was digested with ClaI and EcoRV. A small fragment (−250 base-pairs) was gel purified. Clone 7 (Example 18; SEQ. ID NO: 8) was digested with ClaI and XbaI and a 1400 base-pair fragment was gel purified. The isolated 250 base-pair and 1400 base-pair restriction fragments were ligated to form a fragment of ˜1650 base-pairs.

Due to the single nucleotide substitution in Clone 7 (relative to the 717 base-pair PCR sequence and the original rat sequence) a stop codon at translated base-pair 672 resulted. Because of this stop codon, a truncated (short) protein was encoded, hence the name LMP-1s. This was the construct used in the expression vector (SEQ. ID NO: 32). The full length cDNA sequence with 5′ UTR (SEQ. ID NO: 33) was created by alignment of SEQ. ID NO: 32 with the 5′ RACE sequence (SEQ. ID NO: 21). The amino acid sequence of LMP-1s (SEQ. ID NO: 34) was then deduced as a 223 amino acid protein and confirmed by Western blot (as in Example 15) to run at the predicted molecular weight of ˜23.7 kD.

The pHis-ATG vector (InVitrogen, Carlsbad, Calif.) was digested with EcoRV and XbaI. The vector was recovered and the 650 base-pair restriction fragment ent was then hgated into the linearized pHis-ATG. The ligated product was cloned and amplified. The pHis-ATG-LMP-1s Expression vector, also designated pHIS-A with insert HLMP-1s, was purified by standard methods.

### EXAMPLE 24

### Induction of Bone Nodule Formation and Mineralization In Vitro with LMP Expression Vector

Rat Calvarial cells were isolated and grown in secondary culture according to Example 1. Cultures were either unstimulated or stimulated with glucocorticoid (GC) as described in Example 1. A modification of the Superfect Reagent (Qiagen, Valencia, Calif.) transfection protocol was used to transfect 3, μg/well of each vector into secondary rat calvarial osteoblast cultures according to Example 25.

Mineralized nodules were visualized by Von Kossa staining, as described in Example 3. Human LMP-1s gene product over expression alone induced bone nodule formation (˜203 nodules/well) in vitro. Levels of nodules were approximately 50% of those induced by the GC positive control (˜412 nodules/well). Other positive controls included the pHisA-LMP-Rat expression vector (˜152 nodules/well) and the pCMV2/LMP-Rat-Fwd Expression vector (˜206 nodules/well), whereas the negative controls included the pCMV2/LMP-Rat-Rev. expression vector (˜2 nodules/well) and untreated (NT) plates (˜4 nodules/well). These data demonstrate that the human cDNA was at least as osteoinductive as the rat cDNA. The effect was less than that observed with GC stimulation, most likely due to sub-optimal doses. of Expression vector.

### EXAMPLE 25

### LMP-Induced Cell Differentiation In Vitro and In Vivo

The rat LMP cDNA in clone 10-4 (see Example 12) was excised from the vector by double-digesting the clone with NotI and ApaI overnight at 37° C. Vector pCMV2 MCS (InVitrogen, Carlsbad, Calif.) was digested with the same restriction enzymes. Both the linear cDNA fragment from clone 10-4 and pCMV2 were gel purified, extracted and ligated with T4 ligase. The ligated DNA was gel purified, extracted and used to transform E. coli JM109 cells for amplification. Positive agar colonies were picked, digested with NotI and ApaI and the restriction digests were examined by gel electrophoresis. Stock cultures were prepared of positive clones.

A reverse vector was prepared in analogous fashion except that the restriction enzymes used were XbaI and HindIII. Because these restriction enzymes were used, the LMP cDNA fragment from clone 10-4 was inserted into pRc/CMV2 in the reverse (that is, non-translatable) orientation. The recombinant vector produced is designated pCMV2/RLMP.

An appropriate volume of pCMV 10-4 (60 nM final concentration is optimal [3 μg]; for this experiment a range of 0-600 nM/well [0-30 μg/well] final concentration is preferred) was resuspended in Minimal Eagle Media (MEM) to 450, μl final volume and vortexed for 10 seconds. Superfect was added (7.5 μl/ml final solution), the solution; was vortexed for 10 seconds and then incubated at room temperature for 10 minutes. Following this incubation, MEM supplemented with 10% FBS (1 ml/well; 6 ml/plate) was added and mixed by pipetting.

The resulting solution was then promptly pipetted (1 ml/well) onto washed ROB cultures. The cultures were incubated for 2 hours at 37° C. in a humidified atmosphere containing 5% CO₂. Afterward, the cells were gently washed once with sterile PBS and the appropriate normal incubation medium was added.

Results demonstrated significant bone nodule formation in all rat cell cultures which were induced with pCMV 10-4. For example, pCMV 10-4 transfected cells produced 429 nodules/well. Positive control cultures, which were exposed to Trm, produced 460 nodules/well. In contrast, negative controls, which received no treatment, produced 1 nodule/well. Similarly, when cultures were transfected with pCMV 10-4 (reverse), no nodules were observed.

For demonstrating de novo bone formation in vivo, marrow was aspirated from the hind limbs of 4-5 week old normal rats (rnu/+; heterozygous for recessive athymic condition). The aspirated marrow cells were washed in alpha MEM, centrifuged, and RBCs were lysed by resuspending the pellet in 0.83% NH4CI in 10 mM Tris (pH 7.4). The remaining marrow cells were washed 3× with MEM and transfected for 2 hours with 9, μg of pCMV-LMP-1s (forward or reverse orientation) per 3×10⁶ cells. The transfected cells were then washed 2× with MEM and resuspended at a concentration of 3×10⁷ cells/ml.

The cell suspension (100 μl) was applied via sterile pipette to a sterile 2×5 mm type I bovine collagen disc (Sulzer Orthopaedics, Wheat Ridge, Colo.). The discs were surgically implanted subcutaneously on the skull, chest, abdomen or dorsal, spine of 4-5 week old athymic rats (rnu/rnu). The animals were scarified at 3-4 weeks, at which time the discs, or surgical areas were excised and fixed in 0.70% ethanol. The fixed specimens were analyzed by radiography and undecalcified histologic examination was performed on 5 μm thick sections stained with Goldner Trichrome. Experiments were also performed using devitalized (guanidine extracted) demineralized bone matrix (Osteotech, Shrewsbury, N.J.) in place of collagen discs.

Radiography revealed a high level of mineralized bone formation that conformed to the form of the original collagen disc containing LMP-1s transfected marrow cells. No mineralized bone formation was observed in the negative control (cells transfected with a reverse-oriented version of the LMP-1s cDNA that did not code for a translated protein), and absorption of the carrier appeared to be well underway.

Histology revealed new bone trabeculae lined with osteoblasts in the LMP-1s transfected implants. No bone was seen along with partial resorption of the carrier in the negative controls.

Radiography of a further experiment in which 18 sets (9 negative control pCMV-LMP-REV & 9 experimental pCMV-LMP-1s) of implants were added to sites alternating between lumbar and thoracic spine in athymic rats demonstrated 0/9 negative control implants exhibiting bone formation (spine fusion) between vertebrae. All nine of the pCMV-LMP-1s treated implants exhibited solid bone fusions between vertebrae.

### EXAMPLE 26

### The Synthesis of pHIS-5′ ATG LMP-1s Expression Vector from the Sequences Demonstrated in Examples 2 and 3

The 717 base-pair clone (Example 17) was digested with ClaI and EcoRV (New England Biologicals, city, MA). A small fragment (˜250 base pairs) was gel purified. Clone No. 7 (Example 18) was digested with CIaI and XbaI. A 1400 base-pair fragment was gel purified from that digest. The isolated 250 base-pair and 1400 base-pair cDNA fragments were ligated by standard methods to form a fragment of 1650 bp. The pHis-A vector (InVitrogen) was digested with EcoRV and XbaI. The linearized vector was recovered and ligated to the chimeric 1650 base-pair cDNA fragment. The ligated product was cloned and amplified by standard methods, and the phis-A-5′ ATG LMP-1s expression vector, also denominated as the vector pHis-A with insert HLMP-1s, was deposited at the ATCC as previously described.

### EXAMPLE 27

### The Induction of Bone Nodule Formation and Mineralization In Vitro with pHis-5′ ATG LMP-1s Expression Vector

Rat calvarial cells were isolated and grown in secondary culture according to Example 1. Cultures were either unstimulated or stimulated with glucocorticoid (GC) according to Example 1. The cultures were transfected with 3 μg of recombinant pHis-A vector DNA/well as described in Example 25. Mineralized nodules were visualized by Von Kossa staining according to Example 3.

Human LMP-1s gene product overexpression alone (i.e., without GC stimulation) induced significant bone nodule formation (˜203 nodules/well) in vitro. This is approximately 50% of the amount of nodules produced by cells lo exposed to the GC positive control (˜412 nodules/well). Similar results were obtained with cultures transfected with pHisA-LMP-Rat Expression vector (˜152 nodules/well) and pCMV2/LMP-Rat-Fwd (˜206 nodules/well). In contrast, the; negative control pCMV2ILMP-Rat-Rev yielded (˜2 nodules/well), while approximately 4 nodules/well were seen in the untreated plates. These data demonstrate that the human LMP-1 cDNA was at least as osteoinductive as the rat LMP-1 cDNA in this model system. The effect in this experiment was less than that observed with GC stimulation; but in some the effect was comparable.

### EXAMPLE 28

### LMP Induces Secretion of a Soluble Osteoinductive Factor

Overexpression of RLMP-1 or HLMP-1s in rat calvarial osteoblast cultures as described in Example 24 resulted in significantly greater nodule formation than was observed in the negative control. To study the mechanism of action of LIM mineralization protein conditioned medium was harvested at different time points, concentrated to 10×, sterile filtered, diluted to its original concentration in medium containing fresh serum, and applied for four days to untransfected cells.

Conditioned media harvested from cells transfected with RLMP-1 or HLMP-1s at day 4 was approximately as effective in inducing nodule formation as direct overexpression of RLMP-1 in transfected cells. Conditioned media from cells transfected with RLMP-1 or HLMP-1 in the reverse orientation had no apparent effect on nodule formation. Nor did conditioned media harvested from LMP-1 transfected cultures before day 4 induce nodule formation. These data suggest that expression of LMP-1 caused the synthesis and/or secretion of a soluble factor, which did not appear in culture medium in effective amounts until 4 days post transfection.

Since overexpression of rLMP-1 resulted in the secretion of an osteoinductive factor into the medium, Western blot analysis was used to determine if LMP-1 protein was present in the medium. The presence of RLMP-1 protein was assessed using antibody specific for LMT-1 (QDPDEE) and detected by conventional means. LMP-1 protein was found only in the cell layer of the culture and not detected in the medium.

Partial purification of the osteoinductive soluble factor was accomplished by standard 25% and 100% ammonium sulfate cuts followed by DE-52 anion exchange batch chromatography (100 mM or 500 mM NACl). All activity was observed in the high ammonium sulfate, high NaCl fractions. Such localization is consistent with the possibility of a single factor being responsible for conditioning the medium.

### EXAMPLE 29

### Gene Therapy in Lumbar Spine Fusion Mediated by Low Dose Adenovirus

This study determined the optimal dose of adenoviral delivery of the LMP-1 cDNA (SEQ. ID NO: 2) to promote spine fusion in normal, that is, immune competent, rabbits.

A replication-deficient human recombinant adenovirus was constructed with the LMP-1 cDNA (SEQ. ID NO: 2) driven by a CMV promoter using the Adeno-Quest™ Kit (Quantum Biotechnologies, Inc., Montreal). A commercially available (Quantum Biotechnologies, Inc., Montreal) recombinant adenovirus containing the beta-galactosidase gene was used as a control.

Initially, an in vitro dose response experiment was performed to determine the optimal concentration of adenovirus-delivered LMP-1 (“AdV-LMP-1”) to induce bone differentiation in rat calvarial osteoblast cultures using a 60-minute transduction with a multiplicity of infection (“MOI”) of 0.025, 0.25, 2.5, or 25 plaque-forming units (pfu) of virus per cell. Positive control cultures were differentiated by a 7-day exposure to 109 M glucocorticoid (“GC”). Negative control cultures were left untreated. On day 14, the number of mineralized bone nodules was counted after von Kossa staining of the cultures, and the level of osteocalcin secreted into the medium (pmol/mL) was measured by radioimmunoassay (mean±SEM).

The results of this experiment are shown in Table I, below. Essentially no spontaneous nodules formed in the untreated negative control cultures. The data show that a MOI equal to 0.25 pfu/cell is most effective for osteoinducing bone nodules, achieving a level comparable to the positive control (GC). Lower and higher doses of adenovirus were less effective.

TABLE I

| | Neg. | | Adv-LMP-1 Dose (MOI) | | | |
|---|---|---|---|---|---|---|
| Outcome | Ctrl. | GC | 0.025 | 0.25 | 2.5 | 25 |
| Bone | 0.5 ± 0.2 | 188 ± 35 | 79.8 ± 13 | 145.1 ± 13 | 26.4 ± 15 | 87.6 ± 2 |
| Nodules | | | | | | |
| Osteocalcin | 1.0 ± .1 | 57.8 ± 9 | 28.6 ± 11 | 22.8 ± 1 | 18.3 ± 3 | 26.0 ± 2 |

In vivo experiments were then performed to determine if the optimal in vitro dose was capable of promoting intertransverse process spine fusions in skeletally mature New Zealand white rabbits. Nine rabbits were anesthetized and 3 cc of bone marrow was aspirated from the distal femur through the intercondylar notch using an 18 gauge needle. The buffy coat was then isolated, a 10-minute transduction with AdV-LMP-1 was performed, and the cells were returned to the operating room for implantation. Single level posterolateral lumbar spine arthrodesis was performed with decortication of transverse processes and insertion of carrier (either rabbit devitalized bone matrix or a collagen sponge) containing 8-15 million autologous nucleated buffy coat cells transduced with either AdV-LMP-1 (MOI=0.4) or AdV-BGal (MOI=0.4). Rabbits were euthanized after 5 weeks and spine fusions were assessed by manual palpation, plain x-rays, CT scans, and undecalcified histology.

The spine fusion sites that received AdV-LMP-I induced solid, continuous spine fusion masses in all nine rabbits. In contrast, the sites receiving AdV-BGal, or a lower dose of AdV-LMP-1 (MOI=0.04) made little or no bone and resulted in spine fusion at a rate comparable to the carrier alone (<40%). These results were consistent as evaluated by manual palpation, CT scan, and histology. Plain radiographs, however, sometimes overestimated the amount of bone that was present, especially in the control sites. LMP-1 cDNA delivery and bone induction was successful with both of the carrier materials tested. There was no evidence of systemic or local immune response to the adenovirus vector.

These data demonstrate consistent bone induction in a previously validated rabbit spine fusion model which is quite challenging. Furthermore, the protocol of using autogenous bone marrow cells with intraoperative ex vivo gene transduction (10 minutes) is a more clinically feasible procedure than other methods that call for overnight transduction or cell expansion for weeks in culture. In addition, the most effective dose of recombinant adenovirus (MOI=0.25) was substantially lower than doses; reported in other gene therapy applications (MOI 40-500). We believe this is duelo; the fact that LMP-1 is an intracellular signaling molecule and may have powerful signal amplification cascades. Moreover, the observation that the same concentration of AdV-LMP-1 that induced bone in cell culture was effective in vivo was also surprising given the usual required increase in dose of other growth factors when translating from cell culture to animal experiments. Taken together, these observations indicate that local gene therapy using adenovirus to deliver the LMP-1 cDNA is possible and the low dose required will likely minimize the negative effects of immune response to the adenovirus vector.

### EXAMPLE 30

### Use of Peripheral Venous Blood Nucleated Cells (Buffo Coat) for Gene Therapy with LMP-1 cDNA to Make Bone

In four rabbits we performed spine fusion surgery as above (Example 29) except the transduced cells were the buffy coat from venous blood rather than bone marrow. These cells were transfected with Adeno-LMP or pHIS-LMP plasmid and had equivalent successful results as when bone marrow cells were used. This discovery of using ordinary venous blood cells for gene delivery makes gene therapy more feasible clinically since it avoids painful marrow harvest under general anesthesia and yields two times more cells per mL of starting material.

### EXAMPLE 31

### Isolation of Human LMP-1 Splice Variants

Intron/Exon mRNA transcript splice variants are a relatively common regulatory mechanism in signal-transduction and cellular/tissue development. Splice variants of various genes have been shown to alter protein-protein, protein-DNA, protein-RNA, and protein-substrate interactions. Splice variants may also control tissue specificity for gene expression allowing different forms (and therefore functions) to be expressed in various tissues. Splice variants are a common regulatory phenomenon in cells. It is possible that the LMP splice variants may result in effects in other tissues such as nerve regeneration, muscle regeneration, or development of other tissues.

To screen a human heart cDNA library for splice variants of the HLMP-1 sequence, a pair of PCR primer corresponding to sections of SEQ. ID NO: 22 was prepared. The forward PCR primer, which was synthesized using standard techniques, corresponds to nucleotides 35-54 of SEQ. ID NO: 22. It has the following sequence:
5′ GAGCCGGCATCATGGATTCC 3′ (SEQ. ID NO: 35)

The reverse PCR primer, which is the reverse complement of nucleotides 820-839 in SEQ. ID NO: 22, has the following sequence:
5′GCTGCCTGCACAATGGAGGT 3′ (SEQ. ID NO: 36)

The forward and reverse PCR primers were used to screen human heart cDNA (ClonTech, Cat No 7404-1) for sequences similar to HLMP-1 by standard techniques, using a cycling protocol of 94° C. for 30 seconds, 64° C. for 30 seconds, and 72° C. for 1 minute, repeated 30 times and followed by a 10 minute incubation at 720° C. The amplification cDNA sequences were gel-purified and submitted to the Emory DNA Sequence Core Facility for sequencing. The clones were sequenced using standard techniques and the sequences were examined with PCGENE (intelligenetics; Programs SEQUIN and NALIGN) to determine homology to SEQ. ID NO: 22. Two homologous nucleotide sequences with putative alternative splice sites compared to SEQ. ID NO: 22 were then translated to their respective protein products with Intelligenetic's program TRANSL.

One of these two novel human cDNA sequences (SEQ. ID NO: 37) comprises 1456 bp:

| | |
|---|---|
| cgacgcagag cagcgccctg gccgggccaa gcaggagccg gcatcatgga ttccttcaag | 60 |
| gtagtgctgg aggggccagc accttggggc ttccggctgc aagggggcaa ggacttcaat | 120 |
| gtgcccctct ccatttcccg gctcactcct gggggcaaag cggcgcaggc cggagtggcc | 180 |
| gtgggtgact gggtgctgag catcgatggc gagaatgcgg gtagcctcac acacatcgaa | 240 |
| gctcagaaca agatccgggc ctgcggggag cgcctcagcc tgggcctcag cagggcccag | 300 |
| ccggttcaga gcaaaccgca gaaggtgcag acccctgaca aacagccgct ccgaccgctg | 360 |
| gtcccagatg ccagcaagca gcggctgatg gagaacacag aggactggcg gccgcggccg | 420 |
| gggacaggcc agtcgcgttc cttccgcatc cttgcccacc tcacaggcac cgagttcatg | 480 |
| caagacccgg atgaggagca cctgaagaaa tcaagccagg tgcccaggac agaagcccca | 540 |
| gccccagcct catctacacc ccaggagccc tggcctggcc ctaccgcccc cagccctacc | 600 |
| agccgcccgc cctgggctgt ggaccctgcg tttgccgagc gctatgcccc ggacaaaacg | 660 |
| agcacagtgc tgacccggca cagccagccg gccacgccca cgccgctgca gagccgcacc | 720 |
| tccattgtgc aggcagctgc cggaggggtg ccaggagggg gcagcaacaa cggcaagact | 780 |
| cccgtgtgtc accagtgcca caaggtcatc cggggccgct acctggtggc gttgggccac | 840 |
| gcgtaccacc cggaggagtt tgtgtgtagc cagtgtggga aggtcctgga agagggtggc | 900 |
| ttctttgagg agaagggcgc catcttctgc ccaccatgct atgacgtgcg ctatgcaccc | 960 |
| agctgtgcca agtgcaagaa gaagattaca ggcgagatca tgcacgccct gaagatgacc | 1020 |
| tggcacgtgc actgctttac ctgtgctgcc tgcaagacgc ccatccggaa cagggccttc | 1080 |
| tacatggagg agggcgtgcc ctattgcgag cgagactatg agaagatgtt tggcacgaaa | 1140 |
| tgccatggct gtgacttcaa gatcgacgct ggggaccgct tcctggaggc cctgggcttc | 1200 |
| agctggcatg acacctgctt cgtctgtgcg atatgtcaga tcaacctgga aggaaagacc | 1260 |
| ttctactcca agaaggacag gcctctctgc aagagccatg ccttctctca tgtgtgagcc | 1320 |
| ccttctgccc acagctgccg cggtggcccc tagcctgagg ggcctggagt cgtggccctg | 1380 |
| catttctggg tagggctggc aatggttgcc ttaaccctgg ctcctggccc gagcctgggc | 1440 |
| tcccgggccc tgccca | 1456 |

Reading frame shifts caused by the deletion of a 119 bp fragment (between X) and the addition of a 17 bp fragment (underlined) results in a truncated gene product having the following derived amino acid sequences (SEQ. ID NO: 38):

```
Met Asp Ser Phe Lys Val Val Leu Glu Gly Pro Ala Pro Trp Gly Phe
    1 5 10 15
Arg Leu Gln Gly Gly Lys Asp Phe Asn Val Pro Leu Ser Ile Ser Arg
                          20 25 30
Leu Thr Pro Gly Gly Lys Ala Ala Gln Ala Gly Val Ala Val Gly Asp
                  35 40 45
Trp Val Leu Ser Ile Asp Gly Glu Asn Ala Gly Ser Leu Thr His Ile
          50 55 60
Glu Ala Gln Asn Lys Ile Arg Ala Cys Gly Glu Arg Leu Ser Leu Gly
  65 70 75 80
Leu Ser Arg Ala Gln Pro Val Gln Asn Lys Pro Gln Lys Val Gln Thr
                                  85 90 95
Pro Asp Lys Gln Pro Leu Arg Pro Leu Val Pro Asp Ala Ser Lys Gln
                        100 105 110
Arg Leu Met Glu Asn Thr Glu Asp Trp Arg Pro Arg Pro Gly Thr Gly
                115 120 125
Gln Ser Arg Ser Phe Arg Ile Leu Ala His Leu Thr Gly Thr Glu Phe
        130 135 140
Met Gln Asp Pro Asp Glu Glu His Leu Lys Lys Ser Ser Gln Val Pro
145 150 155 160
Arg Thr Glu Ala Pro Ala Pro Ala Ser Ser Thr Pro Gln Glu Pro Trp
                                165 170 175
Pro Gly Pro Thr Ala Pro Ser Pro Thr Ser Arg Pro Pro Trp Ala Val
                        180 185 190
Asp Pro Ala Phe Ala Glu Arg Tyr Ala Pro Asp Lys Thr Ser Thr Val
                195 200 205
Leu Thr Arg His Ser Gln Pro Ala Thr Pro Thr Pro Leu Gln Ser Arg
        210 215 220
Thr Ser Ile Val Gln Ala Ala Ala Gly Gly Val Pro Gly Gly Gly Ser
225 230 235 240
Asn Asn Gly Lys Thr Pro Val Cys His Gln Cys His Gln Val Ile Arg
                                245 250 255
Ala Arg Tyr Leu Val Ala Leu Gly His Ala Tyr His Pro Glu Glu Phe
                        260 265 270
Val Cys Ser Gln Cys Gly Lys Val Leu Glu Glu Gly Gly Phe Phe Glu
                275 280 285
Glu Lys Gly Ala Ile Phe Cys Pro Pro Cys Tyr Asp Val Arg Tyr Ala
        290 295 300
Pro Ser Cys Ala Lys Cys Lys Lys Lys Ile Thr Gly Glu Ile Met His
305 310 315 320
Ala Leu Lys Met Thr Trp His Val Leu Cys Phe Thr Cys Ala Ala Cys
                                325 330 335
Lys Thr Pro Ile Arg Asn Arg Ala Phe Tyr Met Glu Glu Gly Val Pro
                        340 345 350
Tyr Cys Glu Arg Asp Tyr Glu Lys Met Phe Gly Thr Lys Cys Gln Trp
                355 360 365
Cys Asp Phe Lys Ile Asp Ala Gly Asp Arg Phe Leu Glu Ala Leu Gly
        370 375 380
Phe Ser Trp His Asp Thr Cys Phe Val Cys Ala Ile Cys Gln Ile Asn
385 390 395 400
Leu Glu Gly Lys Thr Phe Tyr Ser Lys Lys Asp Arg Pro Leu Cys Lys
                                405 410 415
Ser His Ala Phe Ser His Val
                        420
```

This 423 amino acid protein demonstrates 100% homology to the protein shown in SEQ. ID NO. 10, except for the sequence in the highlighted ara (amino acids 94-99), which are due to the nucleotide changes depicted above.

The second novel human heart cDNA sequence (SEQ. ID NO: 39) comprises 1575 bp:

| | |
|---|---|
| cgacgcagag cagcgccctg gccgggccaa gcaggagccg gcatcatgga ttccttcaag | 60 |
| gtagtgctgg aggggccagc accttggggc ttccggctgc aagggggcaa ggacttcaat | 120 |
| gtgcccctct ccatttcccg gctcactcct gggggcaaag cggcgcaggc cggagtggcc | 180 |
| gtgggtgact gggtgctgag catcgatggc gagaatgcgg gtagcctcac acacatcgaa | 240 |
| gctcagaaca agatccgggc ctgcggggag cgcctcagcc tgggcctcag cagggcccag | 300 |
| ccggttcaga gcaaaccgca gaaggcctcc gcccccgccg cggaccctcc gcggtacacc | 360 |
| tttgcaccca gcgtctccct caacaagacg gcccggccct ttggggcgcc cccgcccgct | 420 |
| gacagcgccc cgcaacagaa tgggtgcaga cccctgacaa acagccgctc cgaccgctgg | 480 |
| tcccagatgc cagcaagcag cggctgatgg agaacacaga ggactggcgg ccgcggccgg | 540 |
| ggacaggcca gtcgcgttcc ttccgcatcc ttgcccacct cacaggcacc gagttcatgc | 600 |
| aagacccgga tgaggagcac ctgaagaaat caagccaggt gcccaggaca gaagccccag | 660 |
| ccccagcctc atctacaccc caggagccct ggcctggccc taccgccccc agccctacca | 720 |
| gccgcccgcc ctgggctgtg gaccctgcgt ttgccgagcg ctatgccccg gacaaaacga | 780 |
| gcacagtgct gacccggcac agccagccgg ccacgcccac gccgctgcag agccgcacct | 840 |
| ccattgtgca ggcagctgcc ggaggggtgc caggaggggg cagcaacaac ggcaagactc | 900 |
| ccgtgtgtca ccagtgccac aaggtcatcc ggggccgcta cctggtggcg ttgggccacg | 960 |
| cgtaccaccc ggaggagttt gtgtgtagcc agtgtgggaa ggtcctggaa gagggtggct | 1020 |
| tctttgagga gaagggcgcc atcttctgcc caccatgcta tgacgtgcgc tatgcaccca | 1080 |
| gctgtgccaa gtgcaagaag aagattacag gcgagatcat gcacgccctg aagatgacct | 1140 |
| ggcacgtgca ctgctttacc tgtgctgcct gcaagacgcc catccggaac agggccttct | 1200 |
| acatggagga gggcgtgccc tattgcgagc gagactatga gaagatgttt ggcacgaaat | 1260 |
| gccatggctg tgacttcaag atcgacgctg gggaccgctt cctggaggcc ctgggcttca | 1320 |
| gctggcatga cacctgcttc gtctgtgcga tatgtcagat caacctggaa ggaaagacct | 1380 |
| tctactccaa gaaggacagg cctctctgca agagccatgc cttctctcat gtgtgagccc | 1440 |
| cttctgccca cagctgccgc ggtggcccct agcctgaggg gcctggagtc gtggccctgc | 1500 |
| atttctgggt agggctggca atggttgcct taaccctggc tcctggcccg agcctgggct | 1560 |
| cccgggccct gccca | 1575 |

Reading frame shifts caused by the addition of a 17 bp fragment (bolded, italicized and underlined) results in an early translation stop codon at position 565-567 (underlined).

The derived amino acid sequence (SEQ. ID NO: 40) consists of 153 amino acids:

```
Met Asp Ser Phe Lys Val Val Leu Glu Gly Pro Ala Pro Trp Gly Phe
    1 5 10 15
Arg Leu Gln Gly Gly Lys Asp Phe Asn Val Pro Leu Ser Ile Ser Arg
                          20 25 30
Leu Thr Pro Gly Gly Lys Ala Ala Gln Ala Gly Val Ala Val Gly Asp
                  35 40 45
Trp Val Leu Ser Ile Asp Gly Glu Asn Ala Gly Ser Leu Thr His Ile
          50 55 60
Glu Ala Gln Asn Lys Ile Arg Ala Cys Gly Glu Arg Leu Ser Leu Gly
  65 70 75 80
Leu Ser Arg Ala Gln Pro Val Gln Ser Lys Pro Gln Lys Ala Ser Ala
                                  85 90 95
Pro Ala Ala Asp Pro Pro Arg Tyr Thr Phe Ala Pro Ser Val Ser Leu
                        100 105 110
Asn Lys Thr Ala Arg Pro Phe Gly Ala Pro Pro Pro Ala Asp Ser Ala
                115 120 125
Pro Gln Gln Asn Gly Cys Arg Pro Leu Thr Asn Ser Arg Ser Asp Arg
        130 135 140
Trp Ser Gln Met Pro Ala Ser Ser Gly
145 150
```

This protein demonstrates 100% homology to SEQ. ID NO: 10 until amino acid 94, where the addition of the 17 bp fragment depicted in the nucleotide sequence results in a frame shift. Over amino acids 94-153, the protein is not homologous to SEQ. ID NO: 10. Amino acids 154-457 in SEQ. ID NO: 10 are not present due to the early stop codon depicted in nucleotide sequence.

### EXAMPLE 32

### Genomic HLMP-1 Nucleotide Sequence

Applicants have identified the genomic DNA sequence encoding HLMP-1, including putative regulatory elements associated with HLMP-1 expression. The entire genomic sequence is shown in SEQ. ID. NO: 41. This sequence was derived from AC023788 (clone.RP11-564G9), Genome Sequencing Center, Washington University School of Medicine, St. Louis, Mo.

The putative promoter region for HLMP-1 spans nucleotides 2,660-8,733 in SEQ. ID NO: 41. This region comprises among other things, at least ten potential glucocorticoid response elements (“GREs”) (nucleotides 6148-6153, 6226-6231, 6247-6252, 6336-6341, 6510-6515, 6552-6557, 6727-6732, 6752-6757, 7738-7743, and 8255-8260), twelve potential Sma-2 homologues to Mothers against Drosophilla decapentaplegic (“SMAD”) binding element sites (nucleotides 3569-3575, 4552-4558, 4582-4588, 5226-5232, 6228-6234, 6649-6655, 6725-6731, 6930-6936, 7379-7384, 7738-7742, 8073-8079, and 8378-8384), and three TATA boxes (nucleotides 5910-5913, 6932-6935, and 7380-7383). The three TATA boxes, all of the GRES, and eight of the SMAD binding elements (“SBEs”) are grouped in the region spanning nucleotides 5,841-8,733 in SEQ. ID NO: 41. These regulatory elements can be used, for example, to regulate expression of exogenous nucleotide sequences encoding proteins involved in the process of bone formation. This would permit systemic administration of therapeutic factors or genes relating to bone formation and repair, as well as factors or genes associated with tissue differentiation and development.

In addition to the putative regulatory elements, 13 exons corresponding to the nucleotide sequence encoding HLMP-1 have been identified. These exons span the following nucleotides in SEQ. ID NO: 41:

| | |
|---|---|
| Exon 1 | 8733-8767 |
| Exon 2 | 9790-9895 |
| Exon 3 | 13635-13787 |
| Exon 4 | 13877-13907 |
| Exon 5 | 14387-14502 |
| Exon 6 | 15161-15291 |
| Exon 7 | 15401-15437 |
| Exon 8 | 16483-16545 |
| Exon 9 | 16689-16923 |
| Exon 10 | 18068-18248 |
| Exon 11 | 22117-22240 |
| Exon 12 | 22323-22440 |
| Exon 13 | 22575-22911 |

In HLMP-2 there is another exon (Exon 5A), which spans nucleotides 14887-14904.

### EXAMPLE 33

### Expression of HLMP-1 in Intervertebral Disc Cells

LIM mineralization protein-1 (LMP-1) is an intracellular protein that can direct cellular differentiation in osseous and non-osseous tissues. This example demonstrates that expressing human LMP-1 (“HLMP-1”) in intervertebral disc cells increases proteoglycan synthesis and promotes a more chondrocytic phenotype. In addition, the effect of HLMP-1 expression on cellular gene expression was demonstrated by measuring Aggrecan and BMP-2 gene expression. Lumbar intervertebral disc cells were harvested from Sprague-Dawley rats by gentle enzymatic digestion and cultured in monolayer in DMEM/F12 supplemented with 10% FBS. These cells were then split into 6 well plates at approximately 200,000 cells per well and cultured for about 6 days until the cells reached approximately 300,000 cells per well. The culture media was changed to 1% FBS DMEM/F12, and this was considered Day 0.

Replication deficient Type 5 adenovirus comprising a HLMP-1 cDNA operably linked to a cytomegalovirus (“CMV”) promoter has been previously described, for example, in U.S. Pat. No. 6,300,127. The negative control adenovirus was identical except the HLMP-1 cDNA was replaced by LacZ cDNA. For a positive control, uninfected cultures were incubated in the continuous presence of BMP-2 at a concentration of 100 nanograms/milliliter.

On Day 0, the cultures were infected with adenovirus for 30 minutes at 37° C. in 300 microliters of media containing 1% FBS. Fluorescence Activated Cell Sorter (“FACS”) analysis of cells treated with adenovirus containing the green fluorescent protein (“GFP”) gene (“AdGFP”) was performed to determine the optimal dose range for expression of transgene. The cells were treated with adenovirus containing the human LMP-1 cDNA (AdHLMP-1) (at MOIs of 0, 100, 300, 1000, or 3000) or with adenovirus containing the LacZ marker gene (AdLacZ MOI of 1000) (negative control). The culture media was changed at day 3 and day 6 after infection.

Proteoglycan production was estimated by measuring the sulfated glycosaminoglycans (sGAG) present in the culture media (at day 0, 3, and 6) using a di-methyl-methylene blue (“DMMB”) calorimetric assay.

For quantification of Aggrecan and BMP-2 mRNA, cells were harvested at day 6 and the mRNA extracted by the Trizol technique. The mRNA was converted to cDNA using reverse-transcriptase and used for real-time PCR, which allowed the relative abundance of Aggrecan and BMP-2 message to be determined. Real time primers were designed and tested for Aggrecan and BMP-2 in previous experiments. The Cybergreen technique was used. Standardization curves were used to quantitate mRNA abundance.

For transfected cells, cell morphology was documented with a light microscope. Cells became more rounded with AdHLMP-1 (MOI 1000) treatment, but not with AdLacZ treatment. AdLacZ infection did not significantly change cell morphology.

FACS analysis of rat disc cells infected with ADGFP at MOI of 1000 showed the highest percentage cells infected (45%).

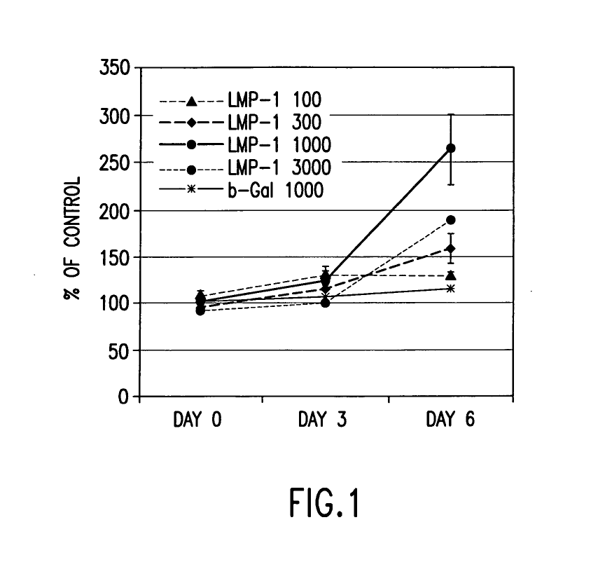

There was a dose dependent increase between sGAG production and AdhLMP-1 MOI. These data are seen in FIG. 1, which shows the production of sGAG after over-expressing HLMP-1 at different MOIs in rat disc cells in monolayer cultures. The results have been normalized to day 0 untreated cells. Error bars represent the standard error of the mean. As shown in FIG. 1, the sGAG production observed at day 3 was relatively minor, indicating a lag time between transfection and cellular production of GAG. Treatment with AdLacZ did not significantly change the sGAG production. As also shown in FIG. 1, the optimal dose of AdhLMP-1 was at a MOI of 1000, resulting in a 260% enhancement of sGAG production over the untreated controls at day 6. Higher or lower doses of AdhLMP-1 lead to a diminished response.

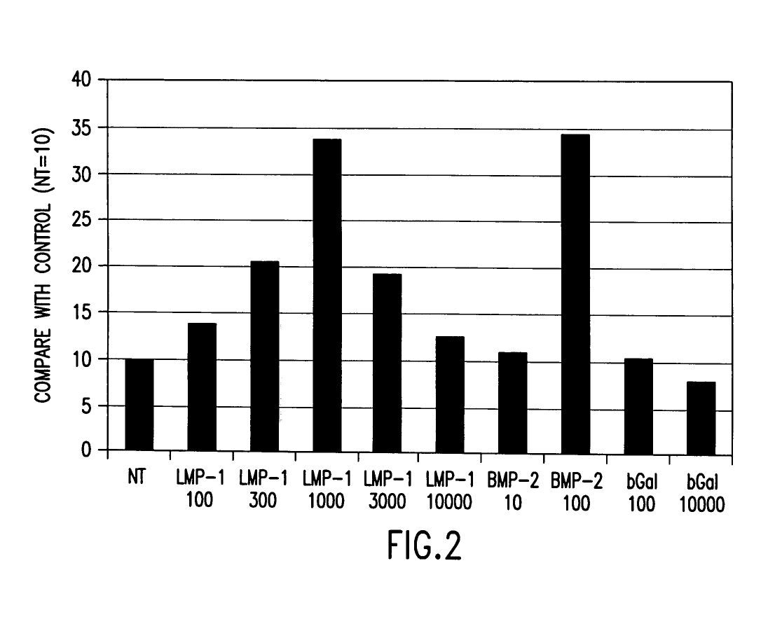

The effect of AdhLMP-1 dosage (MOI) on sGAG production is further illustrated in FIG. 2. FIG. 2 is a chart showing rat disc sGAG levels at day 6 after treatment with AdhLMP-1 at different MOIs. As can be seen from FIG. 2, the optimal dose of AdhLMP-1 was at a MOI of 1000.

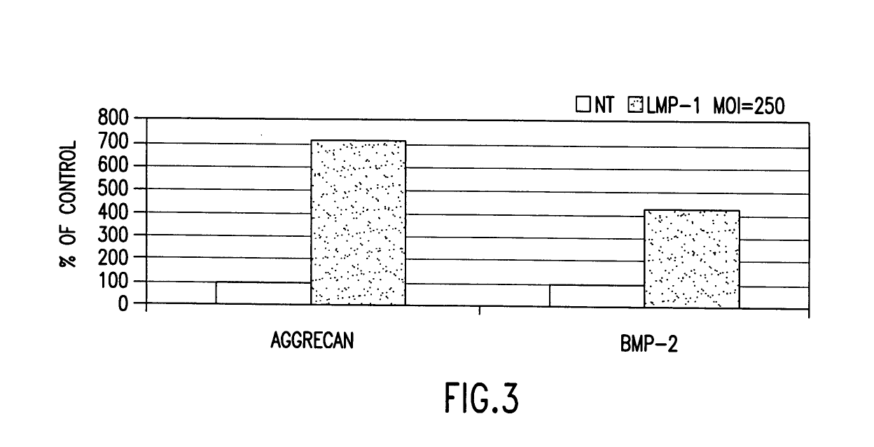

Aggrecan and BMP-2 mRNA production is seen in FIG. 3. This figure demonstrates the increase in Aggrecan and BMP-2 mRNA after over-expression of HLMP-1. Real-time PCR of mRNA extracted from rat disc cells at day 6 was performed comparing the no-treatment (“NT”) cells with cells treated with ADhLMP-1 at a MOI of 250. The data in FIG. 3 are represented as a percentage increase over the untreated sample. As illustrated in FIG. 3, a significant increase in Aggrecan and BMP-2 mRNA was noted following AdhLMP-1 treatment. The increase in BMP-2 expression suggests that BMP-2 is a down-stream gene that mediates HLMP-1 stimulation of proteoglycan synthesis.

These data demonstrate that transfection with AdhLMP-1 is effective in increasing proteoglycan synthesis of intervertebral disc cells. The dose of virus leading to the highest transgene expression (MOI 1000) also leads to the highest induction of sGAG, suggesting a correlation between HLMP-1 expression and sGAG induction. These data indicate that HLMP-1 gene therapy is a method of increasing proteoglycan synthesis in the intervertebral disc, and that HLMP-1 is an agent for treating disc disease.

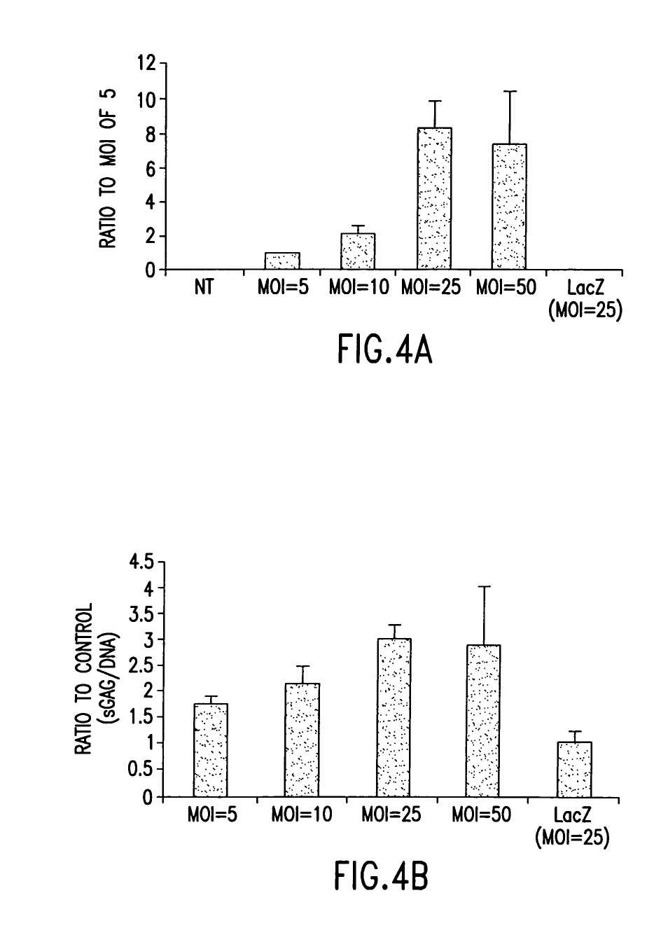

FIG. 4A is a chart showing HLMP-1 mRNA expression 12 hours after infection with Ad-hLMP-1 at different MOIs. In FIG. 4A, exogenous LMP-1 expression was induced with different doses (MOI) of the Ad-hLMP-1 virus and quantitated with realtime PCR. The data is normalized to HLMP-1 mRNA levels from Ad-LMP-1 MOI 5 for comparison purposes. No HLMP-1 was detected in negative control groups, the no-treatment (“NT”) or Ad-LacZ treatment (“LacZ”). HLMP-1 mRNA levels in a dose dependent fashion to reach a plateau of approximately 8 fold with a MOI of 25 and 50.

FIG. 4B is a chart showing the production of sGAG in medium from 3 to 6 days after infection. DMMB assay was used to quantitate total sGAG production between days 3 to 6 after infection. The data in FIG. 4B is normalized to the control (i.e., no treatment) group. As can be seen from FIG. 4B, there was a dose dependent increase in sGAG. with the peak of approximately three fold increase above control reached with a MOI of 25 and 50. The negative control, Ad-LacZ at a MOI of 25, lead to no increase in sGAG. In FIG. 4B, each result is expressed as mean with SD for three samples.

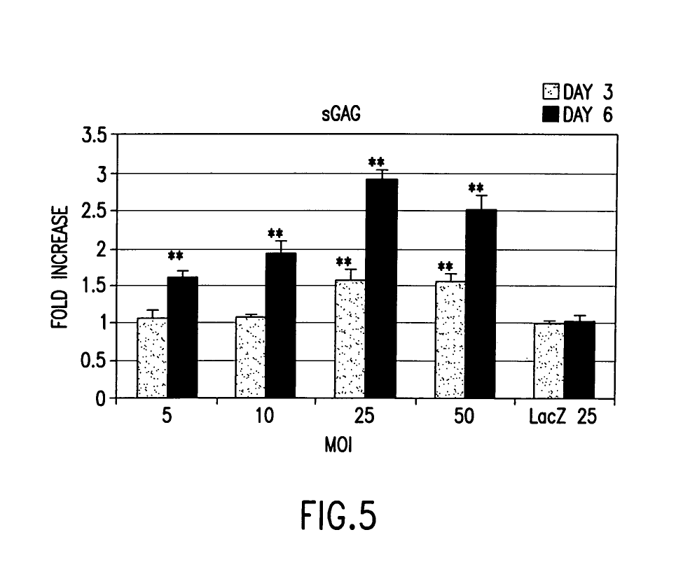

FIG. 5 is a chart showing time course changes of the production of sGAG. As can be seen from FIG. 5, on day 3 sGAG production increased significantly at a MOI of 25 and 50. On day 6 there was a dose dependent increase in sGAG production in response to AdLMP-1. The plateau level of sGAG increase was achieved at a MOI of 25. As can also be seen from FIG. 5, treatment with AdLacZ (“LacZ”) did not significantly change the sGAG production. Each result is expressed as mean with SD for six to nine samples. In FIG. 5, “**” indicates data points for which the P value is <0.01 versus the untreated control.

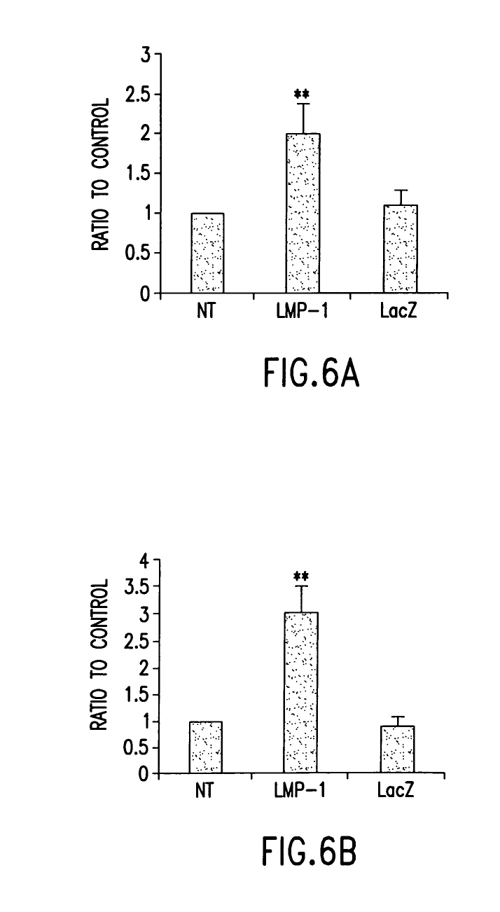

FIGS. 6A and 6B are charts showing gene response to LMP-1 over-expression in rat annulus fibrosus cells for aggrecan and BMP-2, respectively. Quantitative realtime PCR was performed on day 3 after infection with Ad-LMP-1 (“LMP-1”) at a MOI of 25. As can be seen from FIGS. 6A and 6B, the gene expression of aggrecan and BMP-2 increased significantly after infection with Ad-LMT-1 compared to the untreated control (“NT”). Further, treatment with AdLacZ (“LacZ”) at a MOI of 25 did not significantly change the gene expression of either aggrecan or BMP-2 compared to the untreated control. In FIGS. 6A and 6B, each result is expressed as mean with SD for six samples. In FIGS. 6A and 6B, indicates data points for which the P value is P<0.01.

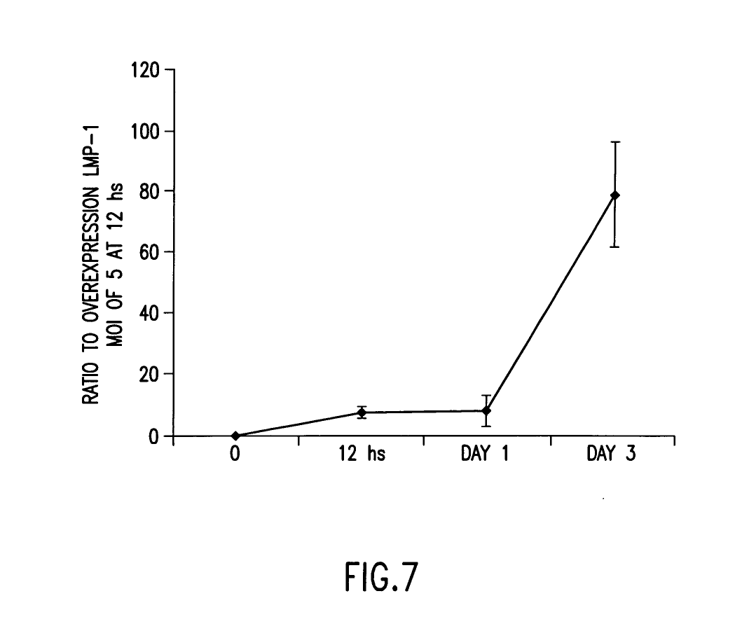

FIG. 7 is a graph showing the time course of HLMP-1 mRNA levels in rat annulus fibrosus cells after infection with AdLMP-1 at a MOI of 25. The data is expressed as a fold increase above a MOI of 5 of AdLMP-1 after standardization using 18S and replication coefficient of over-expression LMP-1 primer. As can be seen from FIG. 7, HLMP-1 mRNA was upregulated significantly as early as 12 hours after infection. Further, there was a marked increase of expression levels between day 1 and day 3. Each result in FIG. 7 is expressed as mean with SD for six samples.

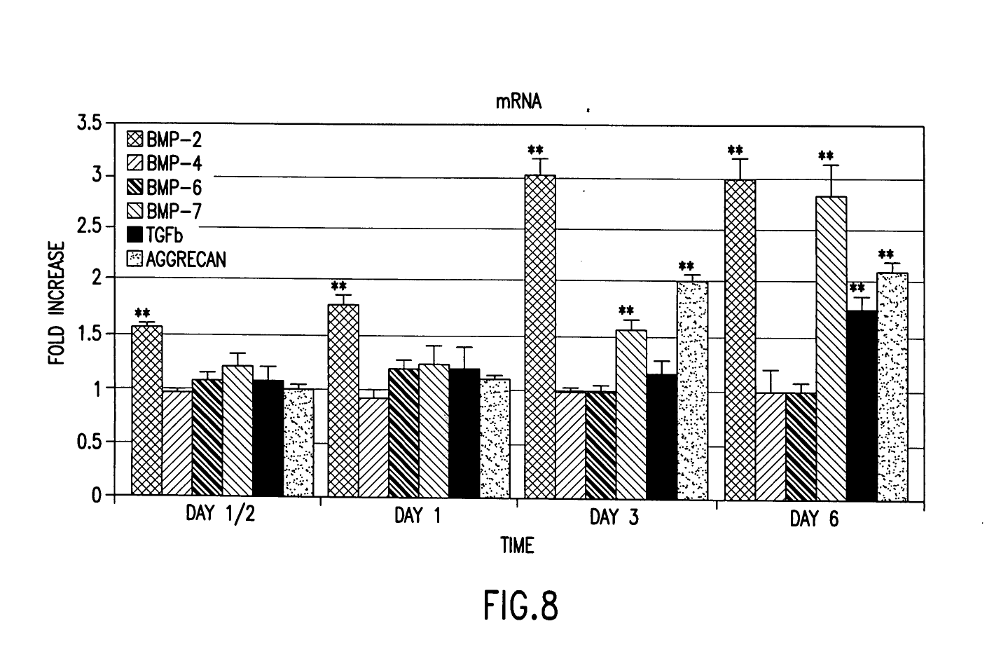

FIG. 8 is a chart showing changes in mRNA levels of BMPs and aggrecan in response to HLMP-1 over-expression. The mRNA levels of BMP-2, BMP-4, BMP-6, BMP7, and aggrecan were determined with realtime-PCR at different time points after infection with Ad-hLMP-1 at a MOI of 25. As can be seen from FIG. 8, BMP-2 mRNA was upregulated significantly as early as 12 hours after infection with AdLMP-1. On the other hand, Aggrecan mRNA was not upregulated until 3 day after infection. Each result is expressed as mean with SD for six samples. In FIG. 8, “**” indicates data points for which the P value is <0.01 for infection with AdLMP-1 versus an untreated control.

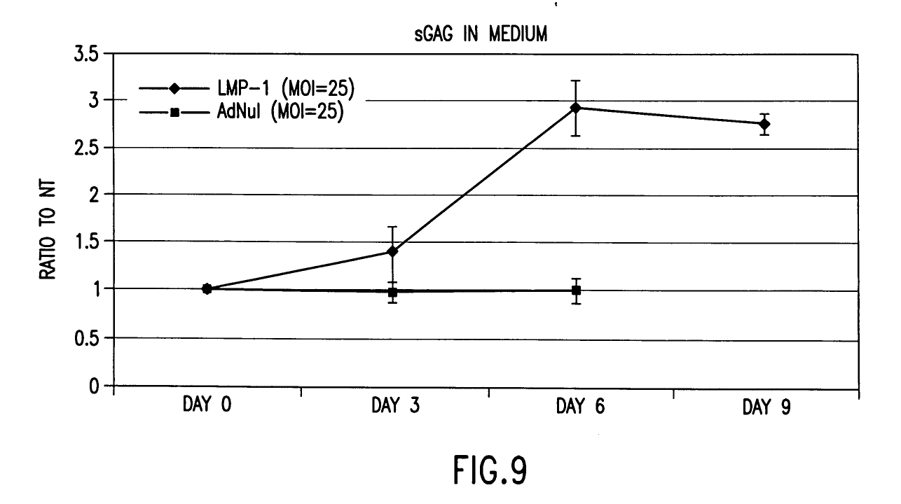

FIG. 9 is a graph showing the time course of sGAG production enhancement in response to HLMP-1 expression. For the data in FIG. 9, rat annulus cells were infected with Ad-hLMP-1 at a MOI of 25. The media was changed every three days after infection and assayed for sGAG with the DMMB assay. This data shows that sGAG production reaches a plateau at day 6 and is substantially maintained at day 9.

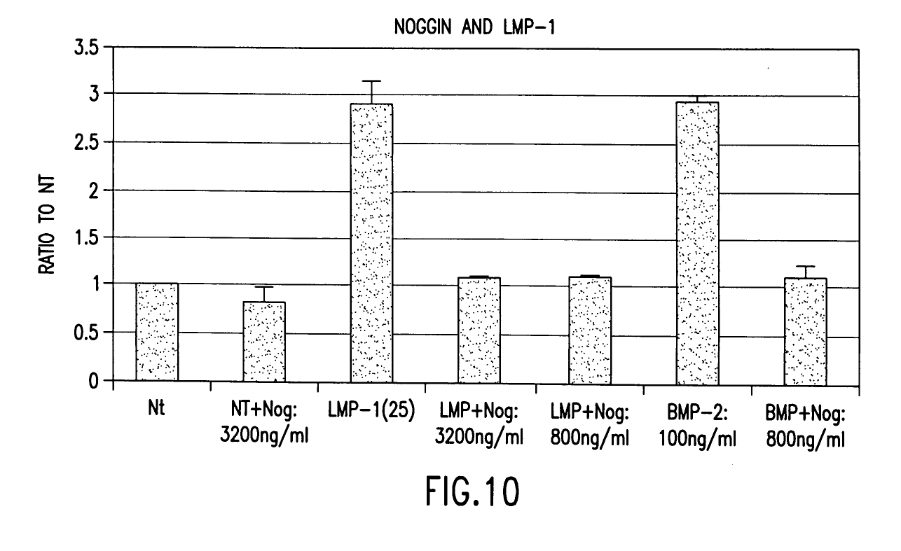

FIG. 10 is a chart showing the effect of noggin (a BMf antagonist) on LMP-1 mediated increase in sGAG production. As seen in FIG. 10, infection of rat annulus cells with Ad-LMP-1 at a MOI of 25 led to a three fold increase in sGAG produced between day 3 and day 6. This increase was blocked by the addition of noggin (a BMP antagonist) at concentration of 3200 ng/ml and 800 nglm. As shown in FIG. 10, however, noggin did not significantly alter sGAG production in uninfected cells. As can also be seen in FIG. 10, stimulation with rhBMP-2 at 100 ng/ml led to a 3 fold increase in sGAG production between day 3 and day 6 after addition of BMP-2. Noggin at 800 ng/ml also blocked this increase.

FIG. 11 is a chart showing the effect of LMP-1 on sGAG in media after day 6 of culture in monolayer. The data points are represented as fold increase above untreated cells. As shown in FIG. 11, LMP-1 with the CMV promoter when delivered by the AAV vector is also effective in stimulating glycosaminoglycan synthesis by rat disc cells in monolayer.

TABLE 2

Primer Sequences for RT-PCR &
Real-time PCR of SYBR Green

| | |
|---|---|
| Primer | Sequence |

| | |
|---|---|
| Aggrecan (forward) | AGGATGGCTTCCACCAGTGC |
| Aggrecan (reverse) | TGCGTAAAAGACCTCACCCTCC |
| BMP-2 (forward) | CACAAGTCAGTGGGAGAGC |
| BMP-2 (reverse) | GCTTCCGCTGTTTGTGTTTG |
| GAPDH (forward) | ACCACAGTCCATGCCATCAC |
| GAPDH (reverse) | TCCACCACCCTGTTGCTGTA |

GAPDH in Table 2 denotes glyceraldehyde phosphate dehydrogenase

TABLE 3

Primer and Probe sequences for
Real-time PCR of TagMan ®

| | |
|---|---|
| Primer | Sequence |

| | |
|---|---|
| Overexpression | AATACGACTCACTATAGGGCTCGA |
| LMP-1 | |
| (forward) | |
| Overexpression | GGAAGCCCCAAGGTGCT |
| LMP-1 | |
| (reverse) | |
| Overexpression | -FAM-AGCCGGCATCATGGATTCCTTCAA-TAMRA |
| LMP-1 (probe) | |

TaqMan® Ribosomal RNA Control Reagents (Part number 4308329, Applied Biosystems, Foster City, Calif., U.S.A.) were used for the forward primer, reverse primer and probe of 18S ribosomal RNA (rRNA) gene.

Mechanism of Bone Formation—Evidence for Induction of Multiple BMPS

Animal and in vitro studies have demonstrated a striking and consistent bone-forming effect with ex vivo gene transfer of the LIM Mineralization Protein-1 (LMP-1) cDNA using relatively low doses of adenoviral or plasmid vectors. See Boden, et al., “Volvo Award in Basic Sciences: Lumbar Spine Fusion by Local Gene Therapy with a cDNA Encoding a Novel Osteoinductive Protein (LMP-1)”, Spine, 23, 2486-2492 (1998); and Viggeswarapu, et al., supra. However, little is known about the mechanism of action of LMP-1, how long the transduced cells survive, or which osteoinductive growth factors and cells participate in the induction of new bone and osteoblast differentiation. See Boden, et al., “LMP-1, A LIM-Domain Protein, Mediates BMP-6 Effects on Bone Formation”, Endocrinology, 139, 5125-5134 (1998). See also Boden. et al., Spine, 23, 2486-2492 (1998), supra, and Viggeswarapu. et al., supra. Furthermore, the mechanism of bone formation in vivo (i.e., endochondral vs. membranous) has not been determined. Understanding the mechanism of LMP-1 action would be helpful for optimal control of LMP-1 induced bone formation in the clinical setting and to further the understanding of intracellular signaling pathways involved with osteoblast differentiation.

LMP-1 is a member of the heterogeneous LIM domain family of proteins and is the first member to be directly associated with osteoblast differentiation. See Kong, et al., “Muscle LIM Protein Promotes Myogenesis by Enhancing the Activity of MyoD.”, Mol. Cell. Biol., 17, 4750-4760 (1997); Sadler, et al., supra; Salgia, et al., supra; and Way, et al., “Mec-3, A Homeobox-Containing Gene that Specifies the Differentiation of the Touch Receptor Neurons in C. Elegans”, Cell, 54, 5-16 (1988). LMP-1 was identified in messenger ribonucleic acid (mRNA) from rat calvarial osteoblasts stimulated by glucocorticoid and later isolated from an osteosarcoma complementary deoxyribonucleic acid (cDNA) library. See Boden et al., Endocrinology, 139, 5125-5134 (1998), supra. Unlike BMPs which are extracellular proteins that act through cell surface receptors, LMP-1 is thought to be an intracellular signaling molecule that is directly involved in osteoblast differentiation. See Boden, et al., Spine, 20, 2626-2632 (1995), supra; Cook, et al., “Effect of Recombinant Human Osteogenic Protein-1 on Healing of Segmental Defects in Non-Human Primates”, J. Bone Joint Surg., 77-A, 734-750 (1995); Schimandle, et al., “Experimental Spinal Fusion with Recombinant Human Bone Morphogenetic Protein-2 (rhBMP-2)”, Spine, 20, 1326-1337 (1995); Spector. et al., “Expression of Bone Morphogenetic Proteins During Membranous Bone Healing”, Plast. Reconstr. Surg., 107, 124-134 (2001); Suzawa, et al., “Extracellular Matrix-Associated Bone Morphogenetic Proteins are Essential for Differentiation of Murine Osteoblastic Cells in vitro”, Endocrinology, 140, 2125-2133 (1999); and Wozney, et al., “Novel Regulators of Bone Formation: Molecular Clones and Activities”, Science, 242, 1528-1534 (1988). Thus, the therapeutic use of LMP-1 may involve gene transfer of its cDNA. On the basis of its association with bone development and the results of suppression and over-expression experiments, LMP is considered to induce secretion of soluble factors that convey its osteoinductive activity, and to be a critical regulator of osteoblast differentiation and maturation in vitro and in vivo. See Boden, et al., Endocrinology, 139, 5125-5134 (1998), supra; Boden, et al., “Differential Effects and Glucocorticoid Potentiation of Bone Morphogenetic Protein Action During Rat Osteoblast Differentiation in vitro”, Endocrinology, 137, 3401-3407 (1996); Knutsen, et al., “Regulation of Insulin-Like Growth Factor System Components by Osteogenic Protein-1 in Human Bone Cells”, Endocrinology, 136, 857-865 (1995); Yeh, et al., “Osteogenic Protein-1 Regulates Insulin-Like Growth Factor-I (IGF-1), IGF-II, and IGF-Binding Protein-5 (IGFBP-5) Gene Expression in Fetal Rat Calvaria Cells by Different Mechanisms”, J. Cell Physiol., 175, 78-88 (1998).

Described below are studies conducted to: 1) to identify candidates for the secreted osteoinductive factors induced by LMP-1; 2) to describe the histologic sequence and type of bone formation induced by LMP-1; and 3) to determine how long the implanted cells overexpressing LMP-1 survive in vivo.

In the present study, human lung carcinoma (A549) cells were used to determine if LMP-1 overexpression would induce expression of bone morphogenetic proteins in vitro. Cultured A549 cells were infected with recombinant replication deficient human type 5 adenovirus containing the LMP-1 or LacZ cDNA. Cells were analyzed using immunohistochemistry after 48 hours. Finally, 16 athymic rats received subcutaneous implants consisting of collagen discs loaded with human buffy coat cells that were infected with one of the above two viruses. Rats were euthanized at intervals and explants analyzed by histology and immunohistochemistry.

Materials and Methods

Phase 1: Detection of LMP-1 induced osteoinductive factors in vitro. The human LMP-1 cDNA with the human cytomegalovirus promoter was cloned into a transfer vector and subsequently was transferred into a recombinant replication deficient (E1, E3 deleted) adenovirus as previously described. See Viggeswarapu, et al., supra.

Human lung carcinoma cells (A549) are known for their high infectivity by human Type 5 adenovirus. These cells were seeded at a density of 50,000 cells/cm², on 2 well chamber slides (Nalge Nunc International, Naperville, Ill.) and were propagated in F12 Kaighn's medium (Gibco BRL), supplemented with 10% fetal bovine serum (PBS), and grown in a humidified 5% CO2 incubator at 37° C.

The A549 cells were infected for 30 minutes at 37° C. on chamber at a multiplicity of infection (MOI) of 10 pfu/cell. Medium with 10% FBS was added and the cells were grown for 48 hours at 37° C. The cells were infected with either AdLMP-1 (active LMP) or AdLacZ (Adpgal-adenovirul control) each driven by the human cytomegalovirus promoter. See Boden, et al., Endocrinology, 139, 5125-5134 (1998), supra; Boden, et al., Spine, 23, 2486-2492 (1998), supra; and Viggeswarapu, et al., supra. As an additional negative, control, some cells were not infected with adenovirus (no treatment control). After 48 hours, the cells on chamber slides were fixed for 2 minutes in 50% acetone/50% methanol, and then were analyzed by immunohistochemistry (described below) using antibodies specific for LMP-1, BMP-2, BMP-4, BMP-6, BMP-7, TGF-131, MyoD, and Type II collagen.

Phase 2: Histologic Sequence of Bone Formation In Vivo

The experimental protocol was reviewed and approved by the Institutional Animal Care and Use Committee and the Human Investigation Committee. Rabbit or human peripheral blood (3 mL) was obtained by venipuncture and the buffy-coat cells were isolated by simple centrifugation at 1200×g for 10 minutes. The cells were counted, and 1×10⁶ cells were infected with adenovirus (AdLMP-1 or AdLacZ) at an MOI of 4.0 pfu/cell for ten minutes at 37° C. After infection, the cells were resuspended in a final volume of 80 uL and applied to a 7 mm×7 mm×3 mm collagen disc (bovine type I collagen).

Sixteen athymic rats that were 4-5 weeks old were obtained (Harlan, Indianapolis, Ind.) and housed in sterile conditions. Rats were anesthetized by inhalation of 1-2% isoflurane. Four 10 mm skin incisions were made on the chest of athymic rats, pockets were developed by blunt dissection, and a collagen disc containing cells was implanted into each pocket. Implants consisted of a collagen disc loaded with buffy coat cells infected with either AdLMP-1 (2 per rat) or AdLacZ (2 per rat). The skin was closed with resorbable suture. Each animal was sacrificed at one, three, five, seven, ten, fourteen, twenty-one and twenty-eight days after implantation; and explants were analyzed by histology and immunohistochemistry.

The specimens were fixed for 24 hours in 10% o neutral buffered formalin. The specimens were prepared for undecalcified or decalcified sectioning. The specimens for undecalcified sections were dehydrated through graded strengths of ethanol and embedded in paraffin. The specimens at 21 and 28 days after implantation were decalcified with 10% ethylenediaminetetraacetic acid (EDTA) solution for 3 to 5 days. After decalcification, the specimens were dehydrated through graded strengths of ethanol and embedded in paraffin. Specimens were sectioned at a thickness of 5 μm on a microtome (Reichert Jung GmbH, Heidelberg, Germany). Sections were subjected to hematoxylin and eosin staining, Goldner's trichrome staining, and immunohistochemical study using antibodies specific for BMP-4, BMP-7, CD-45 and type I collagen.

Preparation of Primary Antibodies

Anti-LMP-1 Antibody: The anti-LMP-1 antibody is an affinity-purified rabbit polyclonal antibody mapping within an internal region of human LMP-1, and reacts with LMP-1 of rabbit and human origin. This antibody was used for the identification of LMP-1 protein at a dilution of 1:500 or 1:1000.

Anti BMP-2, Anti BMP-4, Anti BMP-6, Anti-BMP-7 and Anti-TGF-β1 Antibodies: Polyclonal goat anti-BMP-2, anti-BMP-4, anti-BMP-6, anti-BMP-7, and anti-TGF-β1 antibodies (Santa Cruz Biotechnology, Inc., Santa Cruz, Calif.) cross-react with mouse, rat and human BMPs. The anti-BMP-2, anti-BMP-4 and anti-BMP-6 antibodies were raised against an epitope mapping at the amino terminus of BMP-2, BMP-4 and BMP-6 of human origin. The anti-BMP-7; antibody was an affinity-purified goat polyclonal antibody mapping within an internal region of human BMP-7. The anti-TGF-β1 antibody was an affinity purified goat polyclonal antibody mapping at the carboxy terminus of the precursor form of human TGF-β1. These antibodies were used at a dilution of 1:100 and 1:500 or 1:1000.

Anti-CD45 Antibody: A monoclonal mouse anti-human leukocyte common antigen (LCA), CD-45 antibody (purified IgG 1, kappa; DAKO Co., Carpinteria, Calif.) consists of two antibodies, PD7/26 and 2B11, directed against different epitopes. See Kurtin, et al., supra; and Pulido, et al., supra. The PD7/26 was derived from human peripheral blood lymphocytes maintained on T-cell growth factor. The 2B11 was derived from neoplastic cells isolated from T-cell lymphoma or leukemia. Both antibodies bound to lymphocytes and monocytes at the 94-96 range when tested by immunofluorescence. In the present study, this antibody was used at a dilution of 1:100 for the identification of human leukocytes.

Anti-Collagen Type1 Antibody: A monoclonal anti-type I collagen antibody (mouse IgG 1 isotype; Sigma Chemical Co., Saint Louis, Mo.) was derived from the collagen type I hybridoma produced by the fusion of mouse myeloma cells and splenocytes from BALB/c mice immunized with bovine skin type I Collagen. The antibody reacts with human, bovine, rabbit, deer, pig and rat type I collagen, and was used at a dilution of 1:100.

Anti-Collagen Type II Antibody: A polyclonal rabbit anti-type II collagen antibody (Santa Cruz Biotechnology, Inc., Santa Cruz, Calif.) was raised against an epitope corresponding to the amino terminus of the alpha 1 chain of human type II collagen. The antibody reacts with type II collagen alpha 1 chain of mouse, rat, and human origin and was used at a dilution of 1:1000.

Anti MyoD Antibody: A polyclonal rabbit anti-MyoD antibody (Santa Cruz Biotechnology, Inc., Santa Cruz, Calif.) was raised against an epitope corresponding to amino acids 1-318 representing full length MyoD protein of mouse origin. The antibody reacts with MyoD (and not myogenin, Myf-5, or Myf-6) of mouse, rat, and human origin and was used at a dilution of 1:1000.

Immunohistochemical Staining

The staining procedure was performed using the labeled streptavidin-biotin method (LSAB method). A kit (Universal LSAB Kit, Peroxidase; DAKO Co., Carpinteria, Calif.) was used for immunostaining with antibodies against LMP-1, BMP-2, BMP-4, BMP-6, BMP-7, TGF-β1, CD-45, MyoD, type I collagen, and type II collagen. Appropriate biotinylated secondary antibodies were used depending on the animal in which the primary antibody was raised. Endogenous peroxidase was blocked with methanol containing 0.3% hydrogen peroxide. Specimens were incubated with phosphate buffered saline (PBS) containing either 5% normal rabbit serum or 5% normal goat serum, and 1% bovine serum albumin for 15 minutes at room temperature to avoid nonspecific binding and then with the appropriate concentrations of primary antibodies at 4° C. overnight in a humidified chamber. After washing with PBS three times for 5 minutes, followed by incubation with biotinylated secondary antibody and streptavidin-biotin-peroxiadase complex in a humidified chamber for 10 minutes at room temperature, color was developed using 3,3′-diaminoberzi4 xz., tetrachloride (DAB; DAKO Co., Carpinteria, Calif.). Finally, the sections were counterstained by hematoxylin. As negative controls each primary antibody- was incubated at room temperature for 3 hours with the corresponding blocking peptide (Santa Cruz Biotechnology, Inc., Santa Cruz, Calif.) (1:40 dilution) prior to incubation with the specimens. In some experiments primary antibody alone or secondary antibody alone were used as additional negative controls.

Results

Phase 1: Detection OFLMP-1 Induced Osteoinductive Factors In Vitro.

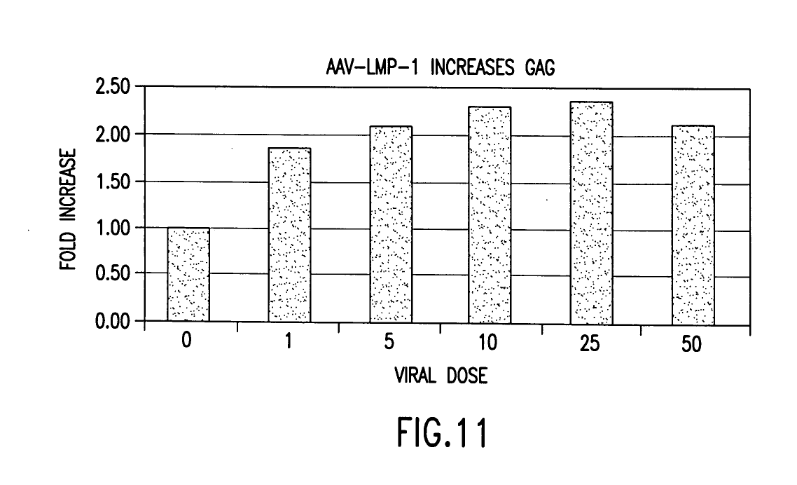

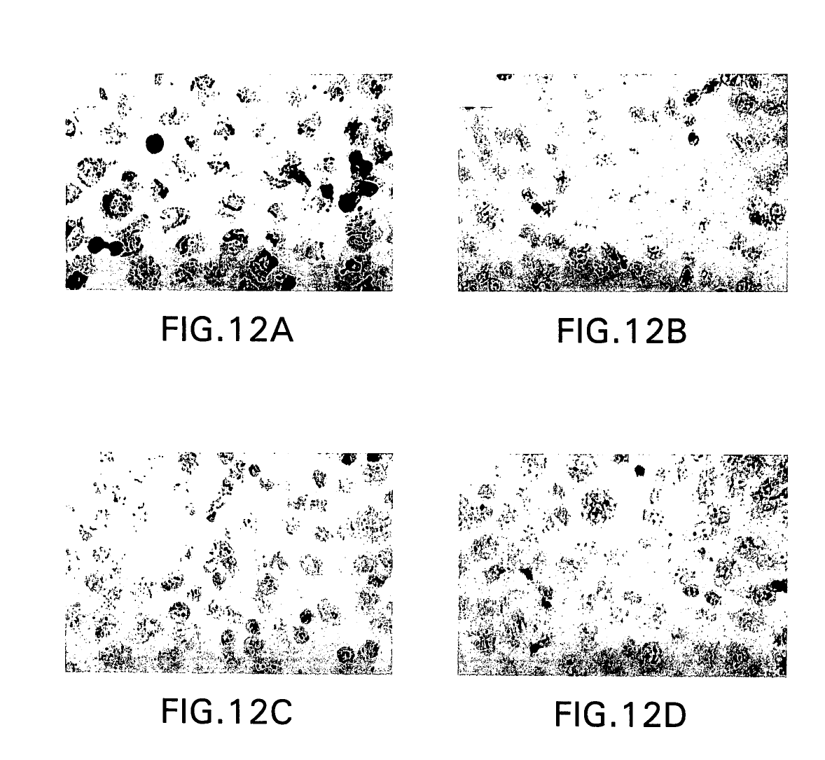

The A549 cells infected with AdLMP-1 showed strong intracellular staining for LMP-1 protein as shown in FIGS. 12A-12D. FIGS. 12A-12D are photomicrographs of immunohistochemical staining for LMT-1 protein in A549 cells 48 hours after infection with AdLMP-1 (FIG. 12A), Adβgal (FIG. 12C), or untreated cells (FIG. 12D). As can be seen from FIGS. 12A, 12C and 12D, a specific intracellular reaction was seen in cells infected with AdLMP-1 (FIG. 12A) but not in either control (FIGS. 12C and 12D). The possibility of non-specific reaction was eliminated since pre-exposure of the primary antibody to a blocking peptide eliminated the positive intracellular staining (FIG. 12B). The photomicrographs of FIGS. 12A-12D were taken at original magnifications of ×132.

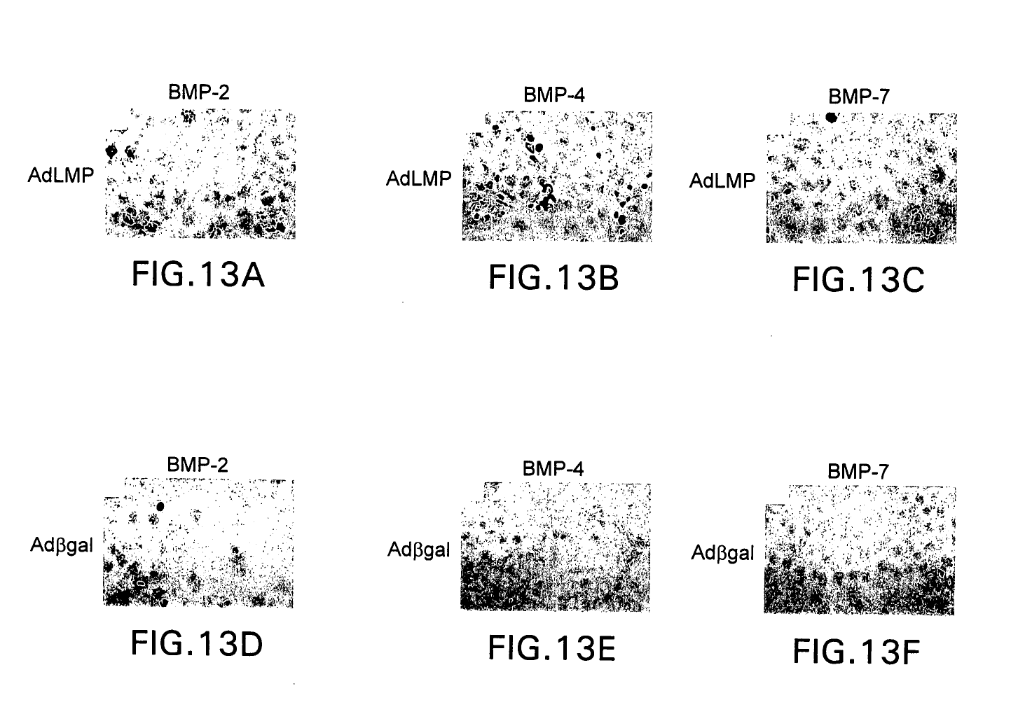

Strong staining for BMP-2, BMP-4 and BMP-7 was observed in the AdLMP-1 treated cells, especially in the cytoplasm, as shown in FIGS. 13A-13F. FIGS. 13A-13F are photomicrographs of immunohistochemical staining of A549 cells 48 hours after infection with AdLMP-1 (upper panels—FIGS. 13A, 13B-and 13C) or Adβgal (lower panels. FIGS. 13D, 13E, and 13F). In AdLMP-1 treated cells there was, specific intracellular staining for BMP-2 (FIG. 13A), BMP-4 (FIG. 13B), and BMP-7 (FIG. 13C) which was not present in Adβgal treated cells (FIGS. 13D, 13E, and 13F, respectively). The photomicrographs of FIGS. 13-13F were taken at original magnifications of ×132.

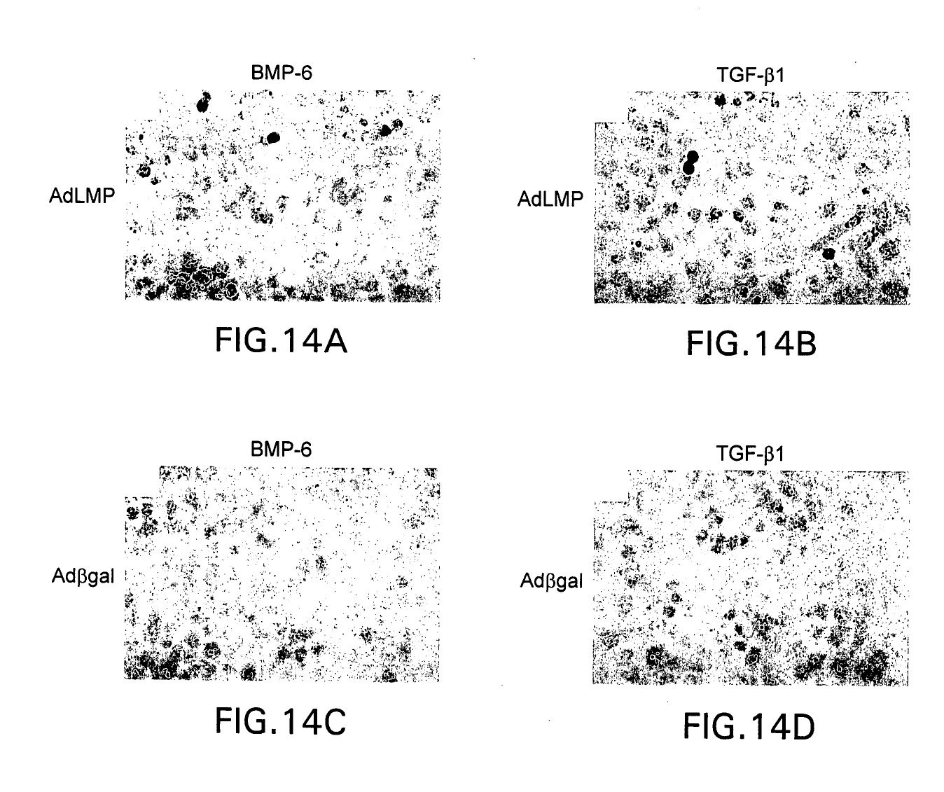

The cells treated with AdLMP-1 also stained positive with anti-BMP-6 and anti-TGF-β1 antibodies as shown in FIGS. 3A-3D. FIGS. 14A-14D are photomicrographs of immunohistochemical staining of A549 cells 48, hours after infection with either AdLMP-1 (upper panels—FIGS. 14A and 14B) or Adβgal (lower panels—FIGS. 14C and 14D). In AdLMP-1 treated cells there was specific intracellular staining for BMP-6 (FIG. 14A) and TGF-β1 (FIG. 14B) which was not present in Adβgal treated cells (FIGS. 14C and 14D, respectively). However, the reactions were somewhat less intense than that seen with other BMPs. In both the Adβgal infected and the untreated controls, the cells had no specific reaction for LMP-1, any of the BMPs, or TGF-β1. A blocking peptide for each antibody confirmed that the reaction was specific. There was no specific reaction with the anti-type II collagen or anti-MyoD antibodies (data not shown). The photomicrographs of FIGS. 14A-14D were taken at an original magnification of ×132.

Phase 2: Histologic Sequence of Bone Formation In Vivo Histological Examination—Immunohistochemical Staining

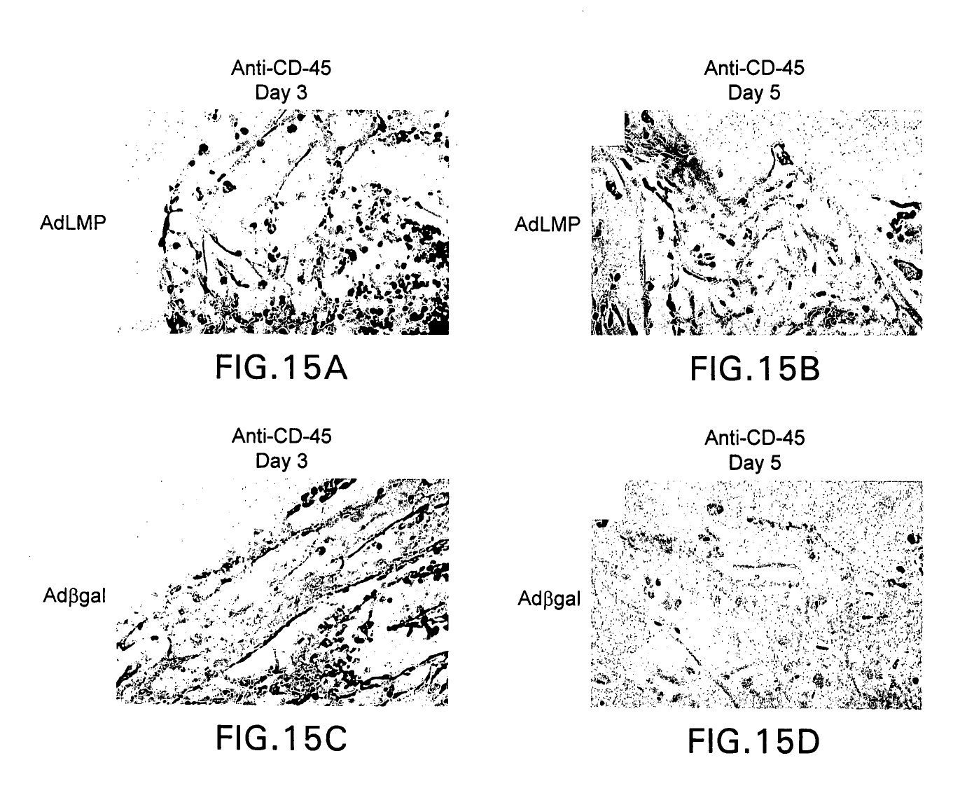

Immunolocalization of leukocytes. At one and three days after implantation, cells stained by anti-CD-45 antibody were abundantly present in huffy coat preparations within both the AdLMP-1 (active) and AdβGA1 (control) treated implants as shown in FIGS. 15A-15D.

FIGS. 15A-15D are photomicrographs of immunohistochemical staining for the leukocyte surface marker CD45 in human buffy coat cells infected with AdLMP-1 (upper panels—FIGS. 15A and 15B) or Adβgal (lower panels—FIGS. 15C and 15D) excised at 3 days (FIGS. 15A and 15C) or 5 days (FIGS. 15B and 15D) following implantation with a collagen matrix subcutaneously on the chest of an athymic rat. The number of cells with specific staining for CD45 antigen decreased rapidly in both treatment groups. This observation suggests that the implanted human cells did not survive very long and the bone formation likely depended on influx of host cells. The number of cells staining with the specific anti-human-CD-45 reaction decreased after Day 3, especially in the center of the implants. Positive staining still was observed in the periphery of the implant at five days, but ten days after implantation there were few cells staining for anti-CD-45. The pattern of decreased staining was the same in active and control implants. The photomicrographs of FIGS. 15A-15D were taken at an original magnification of ×132.

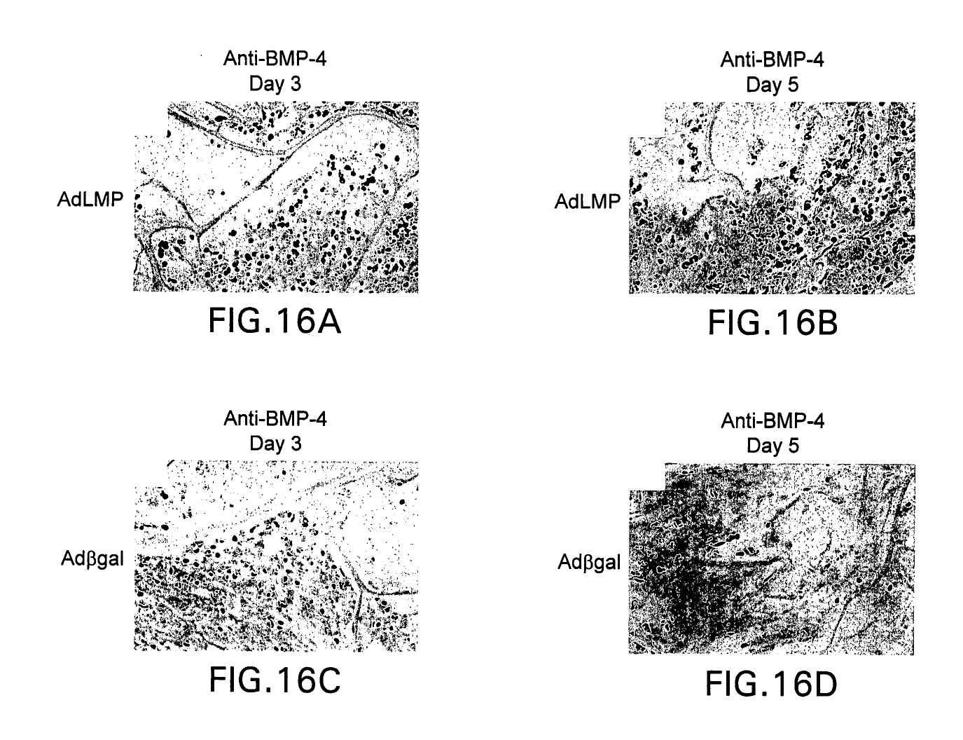

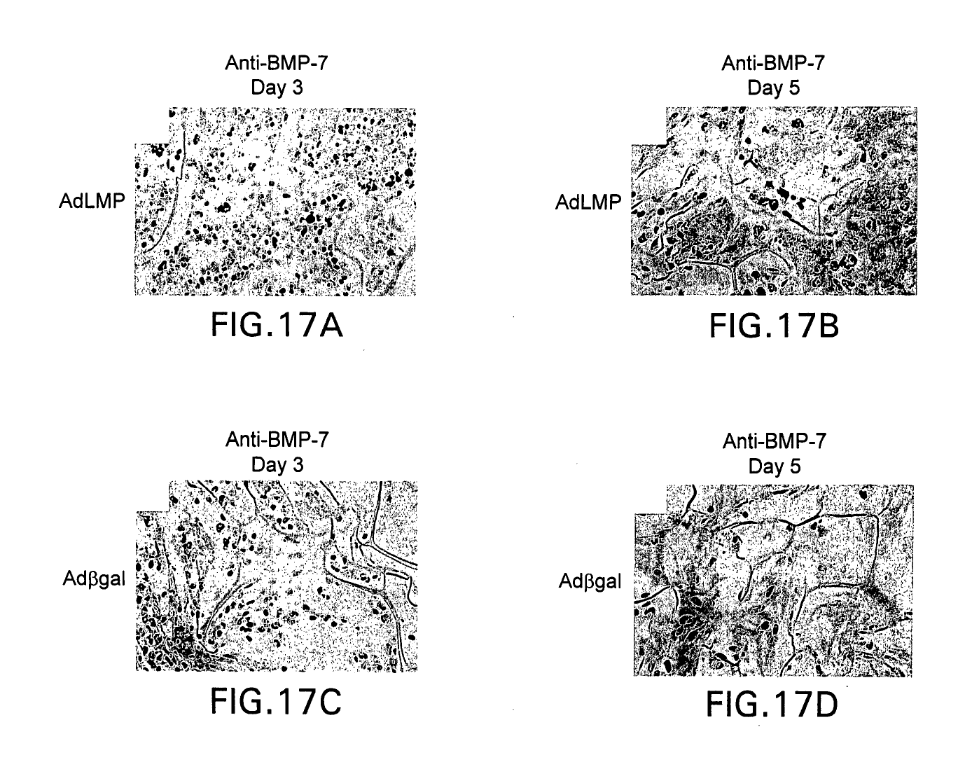

Immunolocalization of BMPS. In the AdLMP-1 treated implants three and five days after implantation, immunohistochemistry revealed strong BMP-4 (FIGS. 16A-16D) and BMP-7 (FIGS. 17A-17D) staining within cells on the collagen fibers.

FIGS. 16A-16D are photomicrographs of immunohistochemical staining for BMP-4 in human buffy coat cells infected with AdLMP-1 (upper pxn.°ls—FIGS. 16A and 16B) or Adogal (lower panels—FIGS. 16C and 16D) excised at 3 days (FIGS. 5A and 5C) or 5 days (FIGS. 5B and 5D) following implantation with a collagen matrix subcutaneously on the chest of an athymic rat. In AdLMP-1 treated cells there was specific intracellular staining for BMP-4 which was not present in Adβgal treated cells. The photomicrographs of FIGS. 16A-16D were taken at an original magnification of ×132.

FIGS. 17A-17D are photomicrographs of immunohistochemical staining for BMP-7 in human buffy coat cells infected with AdLMP-1 (upper panels—FIGS. 17A and 17B) or Adβgal (lower panels—FIGS. 17C and 17D) excised at 3 days (FIGS. 17A and 17C) or 5 days (FIGS. 17B and 17D) following implantation with a collagen matrix subcutaneously on the chest of an athymic rat. In AdLMP-1 treated cells there was specific intracellular staining for BMP-7 which was not present in Adβgal treated cells. The photomicrographs of FIGS. 17A-17D were taken at an original magnification of ×132.

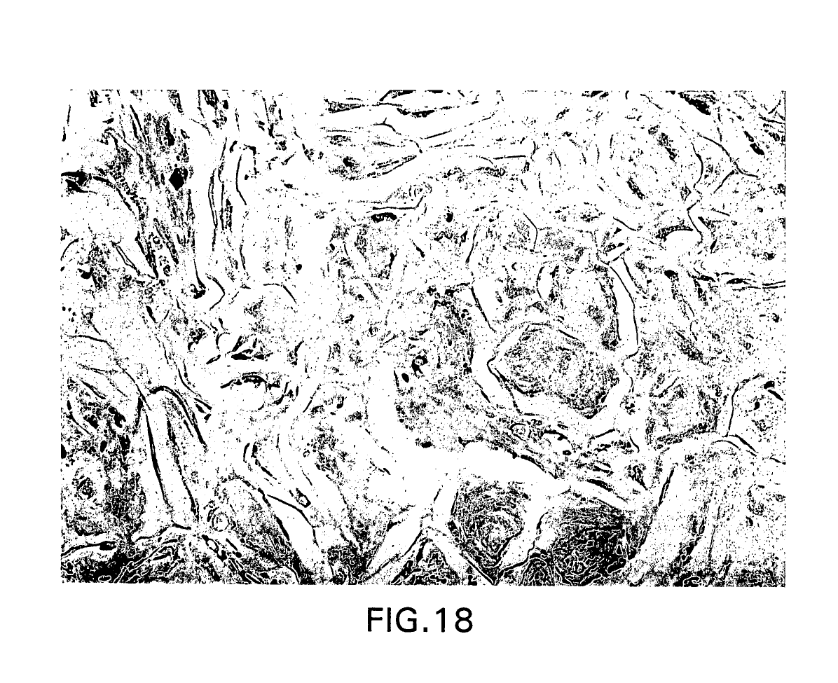

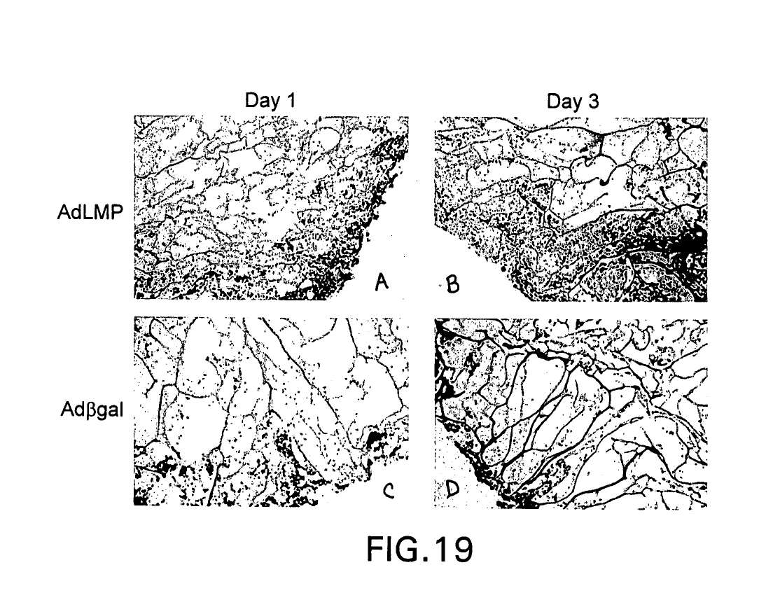

As can be seen from FIGS. 16A-16D and 17A-17D, there was no specific staining for BMP-4 or BMP-7 in cells on the Adβgal (control) implants. Moreover, the strong staining with anti-BMP-4 and anti-BMP-7 antibodies was also seen at each time point beyond 10 days in the AdLMP-1 implants. Strong staining for BMP-4 and BMP-7 was observed in two temporal phases; the first phase was in a limited number of buffy coat cells in the early days (i.e., three and five days after implantation) and the second was seen after Day 10 in osteoblast-like cells surrounded by matrix that most likely were responding cells rather than transplanted huffy coat cells as shown in FIG. 18.

FIG: 18 is a high power photomicrograph of immunohistochemical staining for BMP-7 in human buffy coat cells; infected with AdLMP-I excised at 14 days following implantation with a collageamatrix:subcutaneously on the chest of an athymic rat. There is more abundant staining for BMP-7 compared with earlier time points which is now associated with most of the cells in close proximity to the formation of new bone matrix. The photomicrographs of FIG. 18 was taken at an original magnification of ×66.

Immunolocalization of Type I collagen: Strong staining for anti-type I collagen antibody was observed in the AdLMP-1 implants seven, ten, fourteen, twenty-one and twenty-eight days after implantation. At the early time points, the specific reaction was seen adjacent to osteoblast-like cells and on the periphery of the cells themselves. There was minimal staining for type I collagen in the control implants treated with Adβgal.

Hematoxylin and Eosin & Goldner's Trichrome Staining.

Results were the same whether using rabbit or human buffy coat cells. To avoid duplication, the following description and corresponding illustrations will be for the human donor cells. At one and three days after implantation, the Ad-LMP implants had increased numbers of cells at the edge of the implant as shown in FIGS. 19A-19D.

FIGS. 19A-19D are photomicrographs of human huffy coat cells infected with AdLMP-1 (upper panels—FIGS. 19A and 19B) or Adβgal (lower panels—FIGS. 19C and 19D) excised at 1 day (FIGS. 19A and 19C) or 3 days (FIGS. 19B and 19D) following-implantation in a collagen matrix subcutaneously on the chest of an athymic, rat. The density of cells on the periphery of the implant was greater in the AdLMP-1 implant at both time points suggesting migration of host cells. The photomicrographs of FIGS. 19A-19D were taken at an original magnification of ×33 using Goldner trichrome.

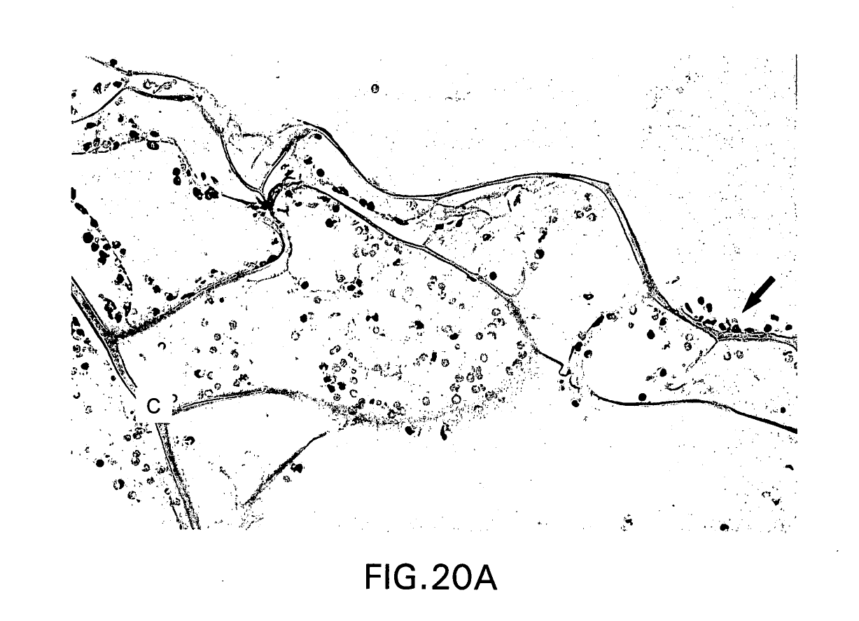

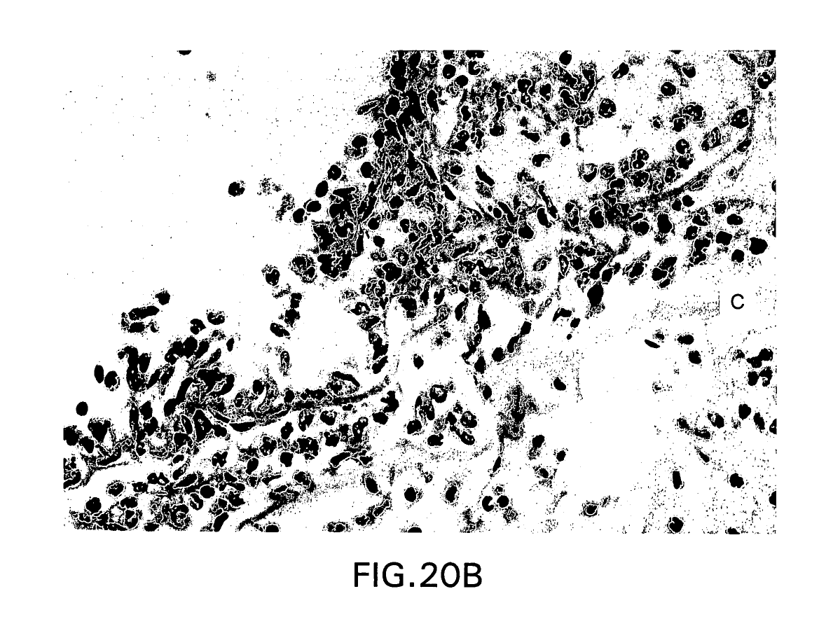

In the Adβgal controls, fewer cells were seen at the periphery at the same time point (i.e., one and three days after implantation). These observations suggest that host cells migrated into the implants with cells expressing LMP-1 as shown in FIGS. 20A and 20B. These cells were a mixture of monocytes and polymorphonuclear leukocytes. FIGS. 20A and 20B are high power photomicrographs of human huffy coat cells infected with AdLMP-1 or Adβgal excised at 1 day following implantation in a collagen matrix subcutaneously on the chest of an athymic rat. As shown in FIG. 20A, there were relatively few cells (arrow) on the periphery of the collagen (C) implants containing cells infected with Adβgal. Buffy coat cells and red cell ghosts could be seen in the center of the implant. As shown in FIG. 20B, the density of nucleated cells on the periphery of the collagen (C) implant was greater in the AdLMP-1 implant suggesting migration of host cells from the surrounding soft tissues. The cells included monocytes, polymorphonuclear cells, and histiocyte appearing cells. The photomicrographs of FIGS. 20A and 20B were taken at original magnifications of ×100 (FIG. 20A) and ×160 (FIG. 20B) using hematoxylin and eosin.

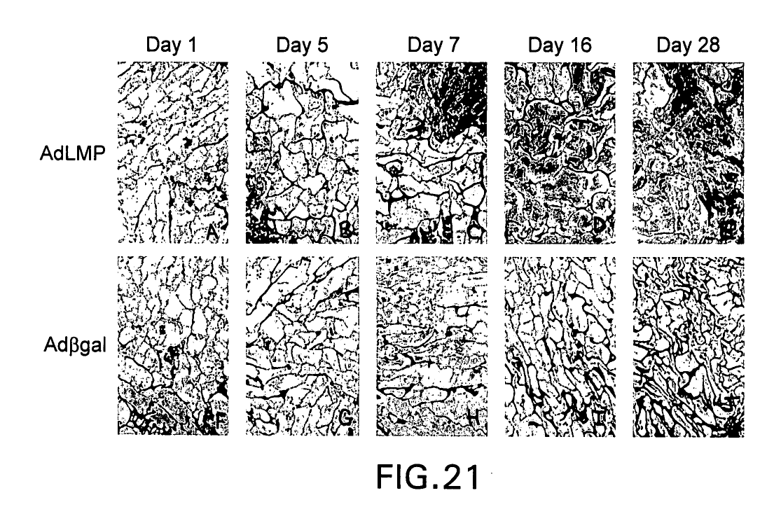

FIGS. 21A-21J are photomicrographs of human buffy coat cells infected with AdLMP-1 (upper panels—FIGS: 21A-21E) or Adβgal (lower panels—FIGS. 21F-21J) excised at various time points following implantation with a collagen matrix subcutaneously on the chest of an athymic rat. The progression of membranous bone formation was evident with mineralized matrix seen by day 7 (FIG. 21C). No bone formation was seen in implants containing cells infected with Adβgal (FIGS. 21F-21J). The photomicrographs of FIGS. 21A-21J were taken at original magnifications of ×33 using Goldner trichrome.

As shown in FIG. 21A-21E, there were less buffy coat cells associated with the collagen fibers over time, and the number of cells surviving in the center of the Adβgal treated implants was diminished by five days after implantation (FIG. 21C).

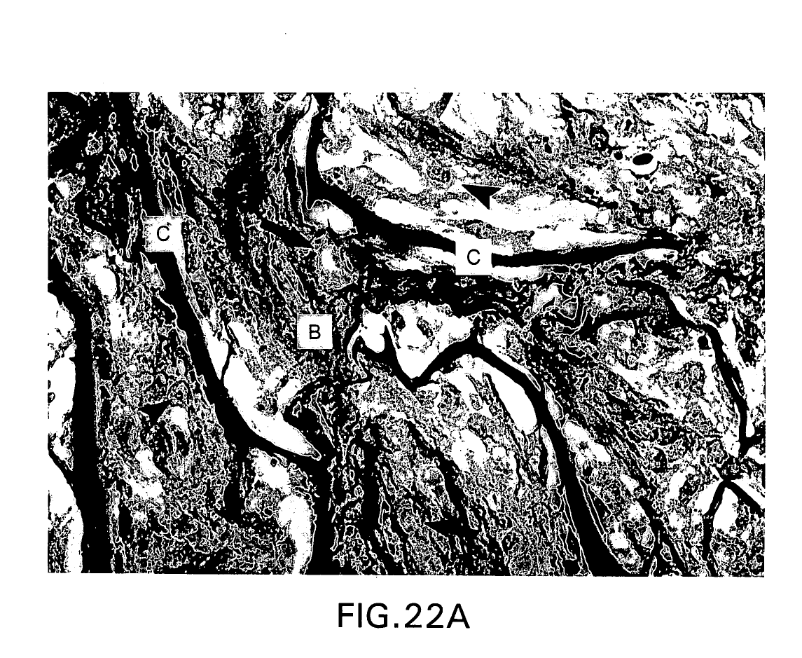

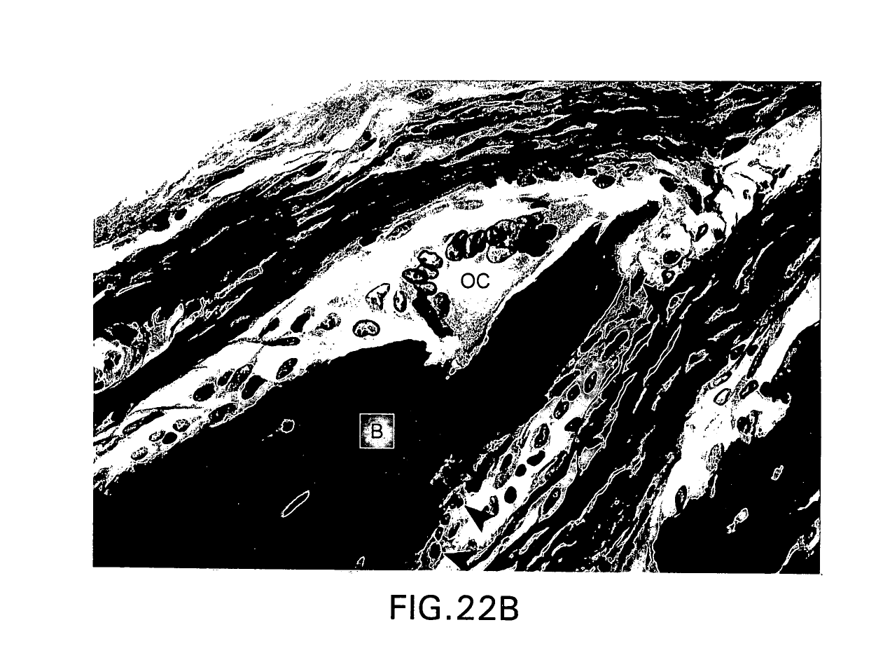

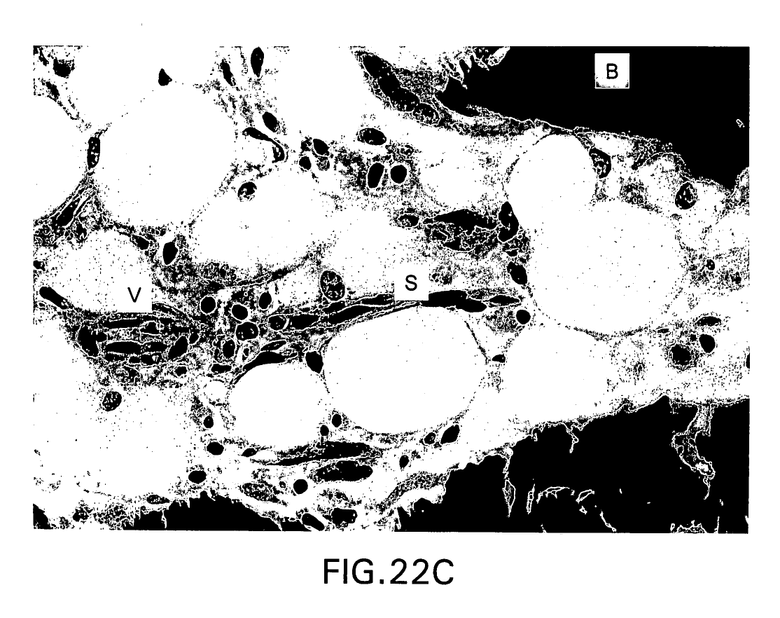

FIGS. 22A-22C are high power photomicrographs of human buffy coat cells infected with AdLMP-1 excised at various time points following implantation with a collagen matrix subcutaneously on the chest of an athymic rat. As can be seen from FIG. 22A, new mineralized bone matrix (B) was visible adjacent to osteoblast-like cells (arrows) between collagen fibers (C) at the periphery of the AdLMP-1 implants seven days after implantation. There was rapid mineralization of the matrix surrounding osteoblast-like cells (arrowheads) without classic osteoid seams and without any specific orientation. As can be seen from FIG. 22B, mature new bone had formed in the spaces located throughout the AdLMP-1 implants and most of the collagen scaffold was resorbed by day 28. Osteoblasts (arrowheads) were seen covering surfaces of osteoid and newly-formed bone while osteoclasts (OC) could be seen remodeling the primary woven bone (B). Finally, as can be seen from FIG. 22C, hematopoietic marrow tissue was also seen forming within the bone (B) including a marrow stroma (S) and blood vessels (V). The photomicrographs of FIGS. 22A-22C were taken at original magnifications of ×160 using Goldner trichrome.

As can be seen from FIG. 22A, new bone matrix was visible adjacent to osteoblast-like cells between collagen fibers at the periphery of the AdLMP-1 implants seven days after implantation. There was rapid mineralization of the surrounding matrix without classic osteoid seams without any specific orientation. The lack of organized bone orientation was not surprising given the fact that these were subcutaneous implants that were not significantly loaded. More abundant osteoblast-like cells were observed in the AdLMP-1 implants ten days after implantation and were growing into the voids between the collagen fibers. By fourteen days after implantation, osteoblast-like cells occupied the central region of the AdLMP-1 implants. In contrast, fibroblast-like cells were filling the voids of the collagen in the Adβgal treated implants. Twenty-one days after implantation, new bone matrix was mineralized and was forming in most or all of the central regions of the AdLMP-1 implants. Mature new bone had formed in the spaces located in the most central regions of the AdLMP-1 implants twenty-eight days after implantation. Osteoblasts were seen covering surfaces of osteoid and newly-formed bone while osteoclasts could be seen remodeling the primary woven bone (FIG. 22B). Hematopoietic marrow tissue was also seen forming within the bone (FIG. 22C). In the Adβgal treated controls, the implanted collagen was mostly resorbed by day 28 and was replaced with fibrous tissue.

As set forth above, in vitro experiments with A549 cells showed that AdLMP-1 infected cells express elevated levels of BMP-2, BMP-4, BMP-6, BMP-7 and TGF-β1 protein. Human huffy coat cells infected with AdLMP-1 also demonstrated increased levels of BMP-4 and BMP-7 protein 72 hours after ectopic implantation in athymic rats, confirming the in vitro hypothesis.

Based on the results of the above study, it has therefore been shown that the osteoinductive properties of LMP-1 involve the synthesis of several BMPs and the recruitment of host cells which differentiate and participate in direct membranous bone formation. Accordingly, gene therapy with the LMP-1 cDNA may provide an alternative to implantation of large doses of single BMPs to induce new bone formation.

According to the invention, a method of inducing the expression of one or more bone morphogenetic proteins or transforming growth factor-β proteins (TGF-βs) in a cell is provided. The method includes transfecting a cell with an isolated nucleic acid comprising a nucleotide sequence encoding a LIM mineralization protein operably linked to a promoter. The expression of one or more proteins selected from the group consisting of BMP-2, BMP-4, BMP-6, BMP-7, TGF-β1 and combinations thereof can be induced according to the invention. The isolated nucleic acid can be a nucleic acid which can hybridize under standard conditions to a nucleic acid molecule complementary to the full length of SEQ. ID NO: 25; and/or a nucleic acid molecule which can hybridize under highly stringent conditions to a nucleic acid molecule complementary to the full length of SEQ. ED NO: 26. The cell can be a buffy coat cell, a stem cell (e.g., a mesenchymal stem cell or a pluripotential stem cell) or an intervertebral disc cell (e.g., a cell of the nucleus pulposus or a cell of the annulus fibrosus). The cell can be transfected ex vivo or in vivo. For example, the cell can be transfected in vivo by direct injection of the nucleic acid into an intervertebral disc of a mammal.

The LIM mineralization protein encoded by the nucleotide sequence can be RLMP, HLMP-1, HLMP-1s, HLMP-2, or HLMP-3. The promoter can be a cytomegalovirus promoter. According to one embodiment of the invention, the LIM mineralization protein is an LMP-1 protein. The nucleic acid can be in a vector (e.g., an expression vector such as a plasmid). The vector can also be a virus such as an adenovirus or a retrovirus. An exemplary adenovirus that can be used according to the invention is AdLMP-1.

According to a second aspect of the invention, a cell which overexpresses one or more bone morphogenetic proteins or transforming growth factor-β proteins is provided. The cell can be a cell which overexpresses one or more proteins selected from the group consisting of BMP-2, BMP-4, BMP-6, BMP-7, TGF-β1 and combinations thereof. The cell can be a buffy coat cell, an intervertebral disc cell, a mesenchymal stem cell or a pluripotential stem cell. An implant comprising a cell as set forth above and a carrier material is also provided. Also provided according to the invention is a method of inducing bone formation in a mammal comprising introducing a cell or an implant as set forth above into the mammal and a method of treating intervertebral disc disease in a mammal comprising introducing a cell as set forth above into an intervertebral disc of the mammal.

Overexpression of a bone morphogenetic protein or a transforming growth factory protein in the context of the invention refers to a cell which expresses that protein at a level greater than normally present in that particular cell (e.g., expression of the protein is at a level greater than the level in a cell which has not been transfected with a nucleic acid comprising a nucleotide sequence encoding a LIM mineralization protein operably linked to a promoter). The cell can be a cell which normally expresses one or more of the bone morphogenetic proteins or transforming growth factory proteins. The cell can also be a cell which does not normally express one or more of the bone morphogenetic proteins or transforming growth factory proteins.

In Vivo Gene Therapy with LMP-1 Causes Upregulation of BMP-2, BMP-7, and Aggrecan mRNA in Rabbit Disc Cells

Intervertebral disc degeneration is associated with the loss of disc nucleus proteoglycan content and a reduction in the rate of newly synthesized proteoglycans (Antoniou et al., J. Clin. Invest. 1996; 98:996-1003; Cs-Szabo et al., Spine 2002 27(20):2212-9). Aggrecan is a high molecular weight proteoglycan that plays a critical role in disc function by increasing nucleus pulposus hydration, and a decrease in aggrecan mRNA level has been noted in the nucleus pulposus of degenerated discs (Cs-Szabo et al., supra). A potential method of preventing or reversing disc degeneration is to increase proteoglycan synthesis by disc cells by means of an anabolic molecule. In vitro studies have shown that both BMP-2 and BMP-7 can stimulate sulfated-proteoglycan synthesis, especially aggrecan (Yoon et al., “The Effect of Bone Morphogenetic Protein-2 on Rat Intervertebral Disc Cells In Vitro”, Spine, Vol. 28, No. 16, pp. 1773-1780, Aug. 15, 2003); Takegami et al., Spine 2002; 27:1318-25). In recent in vitro studies, Lim Mineralization Protein-1 has been shown to stimulate both BMP-2 and BMP-7 from disc cells (Park et al., ORS Transactions, 2002). LMP-1 is a highly conserved intracellular regulatory protein that is important in bone formation. Recently, evidence has been increasing that IMP-1 μlays an important role in cartilage matrix anabolism. Overexpressing LMP-1 in disc cells in vitro with an adenovirus carrying the human LMP-1 gene has been found to increase proteoglycan synthesis through a BMP-2 and BMP-7 mediated-methanism (Park et. al., supra). These in vitro results led us to ask whether overexpressing LMP-1 in vivo can stimulate the synthesis of the regulatory proteins BMP-2 and BMP-7 and the major proteoglycan. aggrecan. We also ask whether LMP-1 is endogenously expressed in rabbit nucleus pulposus tissue.

Methods

Experiment 1: In this preliminary experiment, four New Zealand White rabbits were used. The anterior lumbar discs L2/3, L3/4, L415, and L5/6 were exposed through a left retroperitoneal approach. Replication deficient type 5 adenovirus with the CMV promoter driving either the marker or experimental gene was used (Park et al., supra). The control adenovirus carried the GFP marker gene (AdGFP). The experiment adenovirus carried the human LMP-1 gene (AdLMP-1). Either the AdGFP or AdLMP-1 virus at 107 plaque forming units (pfu) was injected into each of the exposed discs in alternating fashion between AdGFP or AdLMP-1. The adenovirus was delivered in 10 microliters of phosphate buffered saline through a 30G Hamilton syringe. The rabbits were then housed without restriction in individual cages. After 3 weeks, the nucleus pulposus tissues from the injected lumbar discs were harvested. Disc tissues from two rabbits were pooled into control and experimental groups to obtain sufficient mRNA for further analysis. Reverse transcription and real-time PCR were used to quantitate the mRNA levels of total LMP-1 (rabbit and human), BMP-7, and aggrecan. All data are expressed as percent increase over the control (AdGFP group).

Experiment. 2: In this experiment different doses of the AdLMP-1, virus were tested-inuan attempt to establish a dose response relationship. AdLMP-1 at three different doses (106, 107, 108 pfu) and AdGFP at a single dose (107 pfu) (control) were tested. In this experiment, all the discs in one animal were injected with a single virus of the same dose. Two rabbits were used for each virus condition resulting in a total of eight rabbits. One of the AdGFP rabbits died after surgery from unknown causes. The surviving rabbits were then euthanized three weeks later and the mRNA from the discs were harvested. The discs from one rabbit were pooled. Reverse transcription and real-time PCR were used to quantitate the mRNA levels of total LMP-1, overexpressed LMP-1 (human), BMP-2, BMP-7, and aggrecan. All data are expressed as percent increase over the control (AdGFP group).

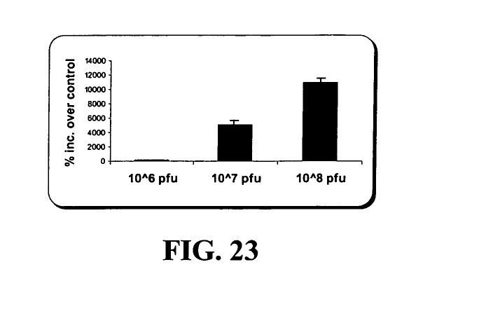

Error bars on FIGS. 23-25 represent one SEM.

Results

Experiment 1 demonstrated that mRNA levels of the discs injected with AdLMP-1 expressed 830% higher levels of total LMP-1 mRNA than the discs injected with AdGFP. A measurable level of endogenous LMP-1 mRNA was detected in the control discs. This was used to calculate the increase in total LMT-1 mRNA in the AdLMP-1 injected discs. BMP-7 mRNA level was increased by 1100% over control. Aggrecan mRNA level was increased by 66% over control.

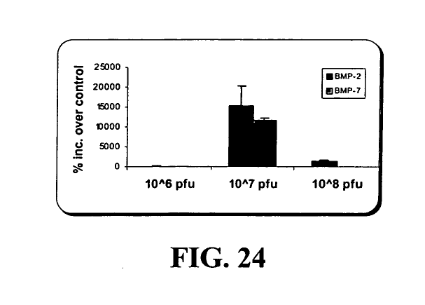

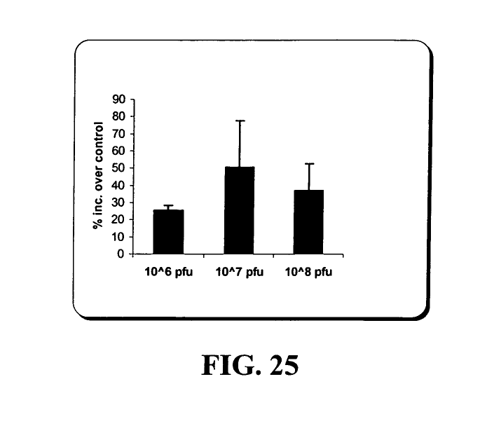

Experiment 2 demonstrated a correlation between increasing AdLMP-1 dose and total LMP-1 mRNA (FIG. 23). Overexpressed LMP-1 (human) mRNA was expressed in a similar pattern to the data seen in FIG. 1, and no expression was se pp in the control group. The BMP-2 and BMP-7 mRNA levels were increased-most highly at a dose of 107 pfu per disc of AdLMP-1 (FIG. 24). Aggrecan mRNA level was most increased with AdLMP-1 at 107 pfu per disc with an increase of 50% over control (FIG. 25).

Discussion

The results show that overexpression of human LMP-1 by in vivo gene therapy with an adenoviral vector is capable of upregulating BMP-2, BMP-7, and aggrecan mRNA. These findings confirm the predictions of our previous short term monolayer culture experiments and represent a major step towards long term in vivo experiments to alter the course of disc degeneration. The endogenous expression of LMP-1 suggests a physiologic role of LMP-1 as a regulator of BMPs that in turn control matrix synthesis by disc cells.

LMP-1 Upregulates Proteoglycans Production and Gene Expression of BMP-2 in Degenerated Human Cervical Disc Cells

Intervertebral disc degeneration of the cervical and lumbar spine is associated with axial pain and other degenerative spinal conditions such as facet arthropathy and stenosis. The pathobiology of intervertebral disc degeneration is characterized by a loss of water and proteoglycan content in the disc (Antoniou et al., supra; Cs-Szabo et al., supra). Transfer of genes encoding for growth factors that might inhibit or reverse these biologic processes may afford an opportunity to prevent or retard disc degeneration. The LMP-I cDNA has been shown to upregulate proteoglycan synthesis though a BMP mediated pathway in disc cells harvested from normal lumbar rat discs (Park et al., supra). While these rat studies were compelling, the effect on degenerated human cervical disc cells was not known. Furthermore, the rat experiments were conducted with the type 5 adenovirus, which is a strain that a significant number of humans have a preimmunity against. Therefore we chose to ask the following three questions: 1) can the chimeric type 5 adenovirus with serotype 35 fiber (type 5/F35 adenovirus), which has a much lower level of human preimmunity, be used to overexpress LMP-1 in disc cells?; 2) Can annulus fibrosus and nucleus pulposus cells from degenerated human cervical discs upregulate BMP-2 mRNA? 3) Can these cells upregulate proteoglycan synthesis in response to LMT-1 stimulation?

Methods

The human LMP-1 cDNA, driven by the CMV promoter, was incorporated into the type 5/35F adenovirus, a replication deficient recombinant adenovirus, to produce our working adenoviral construct (AdLMP-1). This chimeric adenovirus is capable of infecting human cells through a mechanism independent of the CAR receptor and is thought to have higher infectivity. IRB approval was obtained to use disc material that would ordinarily be thrown away. Degenerative intervertebral disc tissue was collected from 2 patients undergoing ACDF for disc herniation and cervical radiculopathy. The discs used in this experiment were clearly degenerated on T2 weighted sagittal MRI views. Annulus fibrosus (AF) and nucleus pulposus (NP) tissue were separately harvested at the time of surgery. The cells were extracted from tissues using Pronase (0.02%) for one hour then Collagenase P (0.0025%) over night. Cells were cultured at 37° C. and 5% CO2 in standard DMEM/F12 with 10% FBS, L-glutamine, L-ascorbic acid, Penicillin, Streptomycin and Amphotericin for 14 to 25 days with media exchange every 2-3 days. Cell-viability was determined by trypan blue exclusion. Cells were then containing the green fluorescent protein gene (AdGFP) instead of the LMT-1 gene served as a negative control. Cultures treated without any virus served as the no-treatment (NT) control. A viral dose of multiplicity of infection (MOI) 10 was used for transfections, as this was established as the optimal dose in previous experiments (data not shown). At Day 6 after viral exposure, the cells were harvested and RNA was isolated. Real time PCR was used to quantify the expression level of specific mRNA (LMP-1 and BMP-2). Media were evaluated at day 3 and 6 for proteoglycan levels by DMMB assay. The proteoglycan level was normalized to the cell number (DNA content) of the culture as measured by the Hoechst dye assay. All experiments were performed in triplicate and repeated at least twice to insure reproducibility. Two-tailed student's t-test was used to calculate p value. P<0.01 was used as criteria for statistical significance.

Results

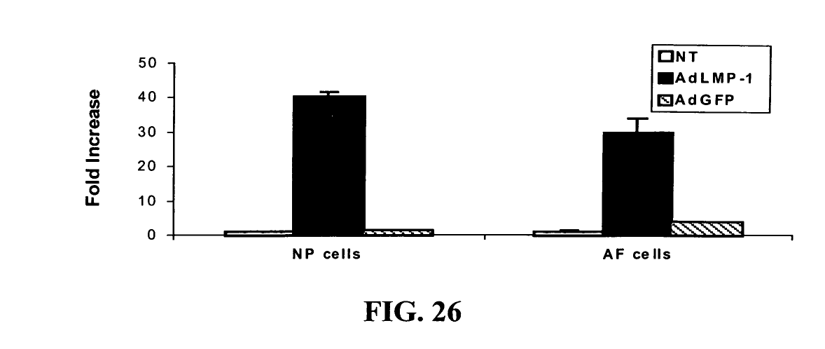

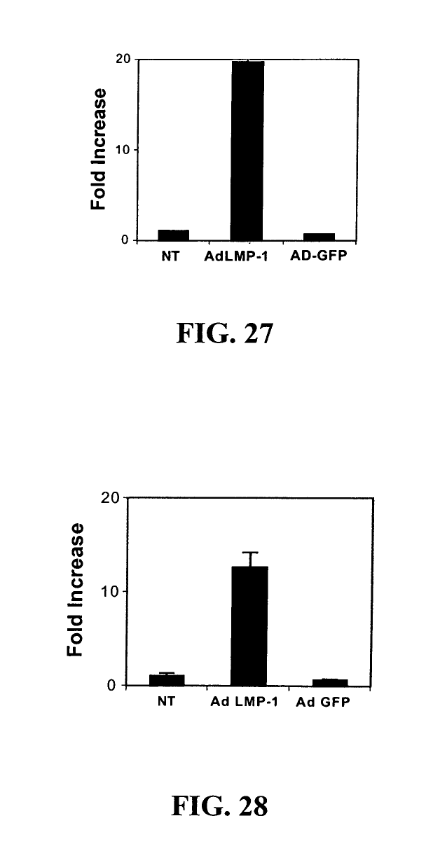

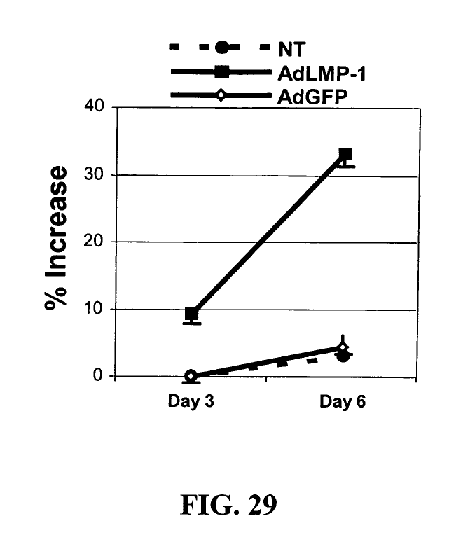

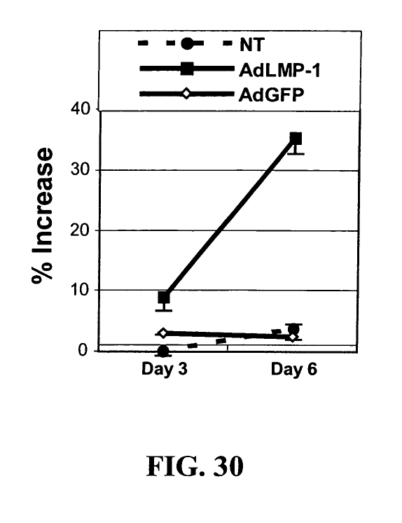

Viable cells were isolated and cultured from human degenerative cervical disc from both annulus fibrosus and nucleus pulposus tissues. Cell viability remained high throughout the incubation and transfection periods (>95%). Basal low level of LMP-1 mRNA expression was found in the non-treatment (NT) and control virus (AdGFP) treated groups. The LMP-1 mRNA level was significantly increased by 40 fold (p<0.01) in AdLMP-1 infected NP cells and by 29 fold (p<0.01) in AdLMP-1 infected AF cells as compared to controls (FIG. 26). The BMP-2-mRNA levels were increased by approximately 20 fold (p<0.01) in nucleus pulposus (FIG. 27) and 12.5 fold (p<0.01) in annulus fibrosus (FIG. 28) cells treated with AdLMP-1. The proteoglycan levels in cell cultures treated with AdLMP-1 were increased by 35% six days after transfection as compared to controls for both NP cells (p<0.01) (FIG. 29) and AF cells (p<0.01) (FIG. 30). There was a minimal rise in proteoglycan levels at day 3.

Discussion

This study confirms that disc cells from degenerated human cervical intervertebral discs can be transfected with the type 5/35F adenovirus to induce expression of potentially therapeutic genes. The upregulation of BMP-2 mRNA and proteoglycan production in response to overexpression of LMP-1 indicate that even cells from degenerated discs can upregulate stimulatory cytokines and increase their anabolic activity. This finding represents an important step in the development of a clinically useful gene therapy for disc degeneration.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A method of inducing or increasing proteoglycan synthesis in a cell, the method comprising:
transfecting the cell with an isolated nucleic acid comprising a nucleotide sequence encoding a LIM mineralization protein operably linked to a promoter.

2. The method of claim 1, wherein the synthesis of aggrecan in the cell is induced or increased.

3. The method of claim 2, wherein the isolated nucleic acid:
hybridizes under standard conditions to a nucleic acid molecule complementary to the full length of SEQ. ID NO: 25; or
hybridizes under highly stringent conditions to a nucleic acid molecule complementary to the full length of SEQ. ID NO: 26.

4. The method of claim 1, wherein the cell is an intervertebral disc cell.

5. The method of claim 1, wherein the cell is transfected ex vivo.

6. The method of claim 1, wherein the cell is transfected in vivo.

7. The method of claim 1, wherein the nucleic acid is in a vector.

8. The method of claim 7, wherein the vector is an expression vector.

9. The method of claim 8, wherein the expression vector is a plasmid.

10. The method of claim 7, wherein the vector is a virus.

11. The method of claim 10, wherein the virus is an adenovirus.

12. The method of claim 11, wherein the adenovirus is a type 5/F35 adenovirus.

13. The method of claim 12, wherein the LIM mineralization protein is hLMP-1.

14. The method of claim 13, wherein the cell is an intervertebral disc cell.

15. The method of claim 14, wherein the cell is a cell of the nucleus pulposus or annulus fibrosus.

16. The method of claim 14, wherein the cell is transfected ex vivo.

17. The method of claim 14, wherein the cell is transfected in vivo.

18. The method of claim 14, wherein the cell is transfected in vivo by direct injection of the adenovirus into an intervertebral disc of a mammal.

19. The method of claim 14, wherein the cell is transfected ex vivo at a multiplicity of infection (MOI) of 5 to 15.

20. The method of claim 14, wherein the cell is transfected ex vivo at a multiplicity of infection (MOI) of about 10.

21. The method of claim 11, wherein the adenovirus is a type 5 adenovirus.

22. The method of claim 11, wherein the LIM mineralization protein is hLMP-1.

23. The method of claim 22, wherein the cell is transfected in vivo by direct injection of the adenovirus into an intervertebral disc of a mammal.

24. The method of claim 23, wherein at least 10⁶ plaque forming units of AdLMP-1 are injected into the intervertebral disc of the mammal.

25. The method of claim 23, wherein from 10⁶ to 10⁸ plaque forming units of AdLMP-1 are injected into the intervertebral disc of the mammal.

26. The method of claim 23, wherein about 10⁷ plaque forming units of AdLMP-1 are injected into the intervertebral disc of the mammal.

27. The method of claim 1, wherein the promoter is a cytomegalovirus promoter.

28. The method according to claim 1, wherein the LIM mineralization protein is rLMP, hLMP-1, hLMP-1s, or hLMP-3.

29. The method according to claim 1, wherein the LIM mineralization protein is hLMP-1.

30. The method of claim 1, wherein the cell is a stem cell or an intervertebral disc cell.

31. The method of claim 30, wherein the cell is a cell of the nucleus pulposus or a cell of the annulus fibrosus.

32. The method of claim 31, wherein the cell is transfected in vivo by direct injection of the nucleic acid into an intervertebral I disc of a mammal.

33. The method of claim 1, wherein the cell is a mesenchymal stem cell or, a pluripotential stem cell.

34. The method of claim 1, wherein the LIM mineralization protein is hLMP-1.

35. A cell which overexpresses one or more proteoglycans.

36. The cell of claim 22, wherein the cell overexpresses aggrecan.

37. The cell of claim 22, wherein the cell is a buffy coat cell, an intervertebral disc cell, a mesenchymal stem cell or a pluripotential stem cell.

38. An implant comprising the cell of claim 36 and a carrier material.

39. A method of treatment comprising introducing the cell of claim 36 into a mammal.

40. A method of treatment comprising introducing the implant of claim 38 into a mammal.

41. A method of treating intervertebral disc disease in a mammal comprising introducing the cell of claim 36 into an intervertebral disc of the mammal.

42. The method of claim 41, wherein the cell is an intervertebral disc cell, a stem cell or a huffy coat cell.

43. An adenovirus vector comprising a nucleotide sequence encoding a LIM mineralization protein operably linked to a promoter wherein the vector is a type 5/F35 adenovirus vector.

44. The method according to claim 43, wherein the LIM mineralization protein is rLMP, hLMP-1, hLMP-1s, or hLMP-3.


SEQUENCE LISTING (42 sequences)

SEQ ID NO: 1 - protein, 457 aa, Rattus norvegicus
MDSFKVVLEGPAPWGFRLQGGKDFNVPLSISRLTPGGKAAQAGVAVGDWVLSIDGENAGSLTHIEAQNKIRACGERLSLGLSRAQPAQSKPQKALTPPADPPRYTFAPSASLNKTARPFGAPPPTDSALSQNGQLLRQLVPDASKQRLMENTEDWRPRPGTGQSRSFRILAHLTGTEFMQDPDEEFMKKSSQVPRTEAPAPASTIPQESWPGPTTPSPTSRPPWAVDPAFAERYAPDKTSTVLTRHSQPATPTPLQNRTSIVQAAAGGGTGGGSNNGKTPVCHQCHKIIRGRYLVALGHAYHPEEFVCSQCGKVLEEGGFFEEKGAIFCPSCYDVRYAPSCAKCKKKITGEIMHALKMTWHVPCFTCAACKTPIRNRAFYMEEGAPYCERDYEKMFGTKCRGCDFKIDAGDRFLEALGFSWHDTCFVCAICQINLEGKTFYSKKDKPLCKSHAFSHV

SEQ ID NO: 2 - DNA, 1696 bp, Rattus norvegicus
gcacgaggatcccagcgcggctcctggaggccgccaggcagccgcccagccgggcattcaggagcaggtaccatggattccttcaaggtagtgctggagggacctgccccttggggcttccgtctgcaagggggcaaggacttcaacgtgcccctctccatctctcggctcactcctggaggcaaggccgcacaggccggtgtggccgtgggagactgggtactgagtatcgacggtgagaacgccggaagcctcacacacattgaagcccagaacaagatccgtgcctgtggggagcgcctcagcctgggtcttagcagagcccagcctgctcagagcaaaccacagaaggccctgacccctcccgccgaccccccgaggtacacttttgcaccaagcgcctccctcaacaagacggcccggcccttcggggcacccccacctactgacagcgccctgtcgcagaatggacagctgctcagacagctggtccctgatgccagcaagcagcggctgatggagaatactgaagactggcgcccgcggccagggacaggccagtcccgttccttccgcatccttgctcacctcacgggcacagagttcatgcaagacccggatgaggaattcatgaagaagtcaagccaggtgcccaggacagaagccccagccccagcctcaaccataccccaggaatcctggcctggccccaccacccccagccccaccagccgcccaccctgggccgtagatcctgcatttgctgagcgctatgccccagacaaaaccagcacagtgctgacccgacacagccagccagccacacctacgcctctgcagaaccgcacctccatagttcaggctgcagctggagggggcacaggaggaggcagcaacaatggcaagacgcctgtatgccaccagtgccacaagatcatccgcggccgatacctggtagcactgggccacgcgtaccatcctgaggaatttgtgtgcagccagtgtgggaaggtcctggaagagggtggcttcttcgaggagaagggagctatcttttgcccctcctgctatgatgtgcgctatgcacccagctgtgccaaatgcaagaagaagatcactggagagatcatgcatgcgctgaagatgacctggcatgttccctgcttcacctgtgcagcctgcaaaacccctatccgcaacagggctttctacatggaggagggggctccctactgcgagcgagattacgagaagatgtttggcacaaagtgtcgcggctgtgacttcaagatcgatgccggggaccgtttcctggaagccctgggtttcagctggcatgatacgtgttttgtttgcgcaatatgtcaaatcaacttggaaggaaagaccttctactccaagaaggacaagcccctgtgcaagagccatgccttttcccacgtatgagcacctcctcacactactgccaccctactctgccagaagggtgataaaatgagagagctctctctccctcgacctttctgggtggggctggcagccattgtcctagccttggctcctggccagatcctggggctccctcctcacagtcccctttcccacacttcctccaccaccaccaccgtcactcacaggtgctagcctcctagccccagttcactctggtgtcacaataaacctgtatgtagctgtg

SEQ ID NO: 3 - DNA, 260 bp, Rattus norvegicus
ttctacatggaggagggggctccctactgcgagcgagattacgagaagatgtttggcacaaagtgtcgcggctgtgacttcaagatcgatgccggggaccgtttcctggaagccctgggtttcagctggcatgatacgtgttttgtttgcgcaatatgtcaaatcaacttggaaggaaagaccttctactccaagaaggacaagcccctgtgcaagagccatgccttttcccacgtatgagcacctcctcacactactgc

SEQ ID NO: 4 - DNA, 16 bp, MMLV
aagctttttttttttg

SEQ ID NO: 5 - DNA, 13 bp, MMLV
aagcttggctatg

SEQ ID NO: 6 - DNA, 223 bp, Homo sapiens
atccttgctcacctcacgggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccagacaaaaccagcacagtgc

SEQ ID NO: 7 - DNA, 717 bp, Homo sapiens
atggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacg

SEQ ID NO: 8 - DNA, 1488 bp, Homo sapiens
atcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgagctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctgggctcccgggcccctgcccacccaccttatcctcccaccccactccctccaccaccacagcacaccggtgctggccacaccagccccctttcacctccagtgccacaataaacctgtacccagctgaattccaaaaaatccaaaaaaaaa

SEQ ID NO: 9 - DNA, 1644 bp, Homo sapiens
atggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctgggctcccgggcccctgcccacccaccttatcctcccaccccactccctccaccaccacagcacaccggtgctggccacaccagccccctttcacctccagtgccacaataaacctgtacccagctgaattccaaaaaatccaaaaaaaaa

SEQ ID NO: 10 - protein, 457 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGQPLRPLVPDASKQRLMENTEDWRPRPGTGQSRSFRILAHLTGTEFMQDPDEEHLKKSSQVPRTEAPAPASSTPQEPWPGPTAPSPTSRPPWAVDPAFAERYAPDKTSTVLTRHSQPATPTPLQSRTSIVQAAAGGVPGGGSNNGKTPVCHQCHKVIRGRYLVALGHAYHPEEFVCSQCGKVLEEGGFFEEKGAIFCPPCYDVRYAPSCAKCKKKITGEIMHALKMTWHVHCFTCAACKTPIRNRAFYMEEGVPYCERDYEKMFGTKCHGCDFKIDAGDRFLEALGFSWHDTCFVCAICQINLEGKTFYSKKDRPLCKSHAFSHV

SEQ ID NO: 11 - DNA, 22 bp, Rattus norvegicus
gccagggttttcccagtcacga

SEQ ID NO: 12 - DNA, 22 bp, Rattus norvegicus
gccagggttttcccagtcacga

SEQ ID NO: 13 - DNA, 22 bp, Homo sapiens
tcttagcagagcccagcctgct

SEQ ID NO: 14 - DNA, 22 bp, Homo sapiens
gcatgaactctgtgcccgtgag

SEQ ID NO: 15 - DNA, 20 bp, Rattus norvegicus
atccttgctcacctcacggg

SEQ ID NO: 16 - DNA, 22 bp, Rattus norvegicus
gcactgtgctggttttgtctgg

SEQ ID NO: 17 - DNA, 23 bp, Homo sapiens
catggattccttcaaggtagtgc

SEQ ID NO: 18 - DNA, 20 bp, Homo sapiens
gttttgtctggggcagagcg

SEQ ID NO: 19 - DNA, 44 bp, Artificial Sequence
  Description of Artificial Sequence  adaptor for Marathon RACE reactions
ctaatacgactcactatagggctcgagcggccgcccgggcaggt

SEQ ID NO: 20 - DNA, 27 bp, Artificial Sequence
  Description of Artificial Sequence  PCR primer specific for Marathon RACE adaptor
ccatcctaatacgactcactatagggc

SEQ ID NO: 21 - DNA, 765 bp, Homo sapiens
ccgttgtttgtaaaacgacgcagagcagcgccctggccgggccaagcaggagccggcatcatggattccttcaaggtagtgctggaggggccagcaccttggggcttccggctgcaagggggcaaggacttcaatgtgccctcctccatttcccggctcacctctgggggcaaggccgtgcaggccggagtggccgtaagtgactgggtgctgagcatcgatggcgagaatgcgggtagcctcacacacatcgaagctcagaacaagatccgggcctgcggggagcgcctcagcctgggcctcaacagggcccagccggttcagaacaaaccgcaaaaggcctccgcccccgccgcggaccctccgcggtacacctttgcaccaagcgtctccctcaacaagacggcccggcccttgggggcgcccccgcccgctgacagcgccccgcagcagaatggacagccgctccgaccgctggtcccagatgccagcaagcagcggctgatggagaacacagaggactggcggccgcggccggggacaggccagtgccgttcctttcgcatccttgctcaccttacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgcc

SEQ ID NO: 22 - DNA, 1689 bp, Homo sapiens
cgacgcagagcagcgccctggccgggccaagcaggagccggcatcatggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctgggctcccgggcccctgcccacccaccttatcctcccaccccactccctccaccaccacagcacaccggtgctggccacaccagccccctttcacctccagtgccacaataaacctgtacccagctgaattccaaaaaatccaaaaaaaaa

SEQ ID NO: 23 - DNA, 22 bp, Homo sapiens
gcactgtgctcgttttgtccgg

SEQ ID NO: 24 - DNA, 21 bp, Homo sapiens
tccttgctcacctcacgggca

SEQ ID NO: 25 - DNA, 30 bp, Homo sapiens
tcctcatccgggtcttgcatgaactcggtg

SEQ ID NO: 26 - DNA, 28 bp, Homo sapiens
gcccccgcccgctgacagcgccccgcaa

SEQ ID NO: 27 - DNA, 24 bp, Homo sapiens
tccttgctcacctcacgggcaccg

SEQ ID NO: 28 - DNA, 22 bp, Homo sapiens
gtaatacgactcactatagggc

SEQ ID NO: 29 - DNA, 23 bp, Rattus norvegicus
gcggctgatggagaatactgaag

SEQ ID NO: 30 - DNA, 23 bp, Rattus norvegicus
atcttgtggcactggtggcatac

SEQ ID NO: 31 - DNA, 22 bp, Rattus norvegicus
tgtgtcgggtcagcactgtgct

SEQ ID NO: 32 - DNA, 1620 bp, Homo sapiens
atggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgagctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctgggctcccgggcccctgcccacccaccttatcctcccaccccactccctccaccaccacagcacaccggtgctggccacaccagccccctttcacctccagtgccacaataaacctgtacccagctg

SEQ ID NO: 33 - DNA, 1665 bp, Homo sapiens
cgacgcagagcagcgccctggccgggccaagcaggagccggcatcatggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatggacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgagctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctgggctcccgggcccctgcccacccaccttatcctcccaccccactccctccaccaccacagcacaccggtgctggccacaccagccccctttcacctccagtgccacaataaacctgtacccagctg

SEQ ID NO: 34 - protein, 223 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGQPLRPLVPDASKQRLMENTEDWRPRPGTGQSRSFRILAHLTGTEFMQDPDEEHLKKSSQVPRTEAPAPASSTPQEPWPGPTAPSPTSRPP

SEQ ID NO: 35 - DNA, 20 bp, Homo sapiens
gagccggcatcatggattcc

SEQ ID NO: 36 - DNA, 20 bp, Homo sapiens
gctgcctgcacaatggaggt

SEQ ID NO: 37 - DNA, 1456 bp, Homo sapiens
cgacgcagagcagcgccctggccgggccaagcaggagccggcatcatggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggtgcagacccctgacaaacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctgggctcccgggccctgccca

SEQ ID NO: 38 - protein, 423 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQNKPQKVQTPDKQPLRPLVPDASKQRLMENTEDWRPRPGTGQSRSFRILAHLTGTEFMQDPDEEHLKKSSQVPRTEAPAPASSTPQEPWPGPTAPSPTSRPPWAVDPAFAERYAPDKTSTVLTRHSQPATPTPLQSRTSIVQAAAGGVPGGGSNNGKTPVCHQCHQVIRARYLVALGHAYHPEEFVCSQCGKVLEEGGFFEEKGAIFCPPCYDVRYAPSCAKCKKKITGEIMHALKMTWHVLCFTCAACKTPIRNRAFYMEEGVPYCERDYEKMFGTKCQWCDFKIDAGDRFLEALGFSWHDTCFVCAICQINLEGKTFYSKKDRPLCKSHAFSHV

SEQ ID NO: 39 - DNA, 1575 bp, Homo sapiens
cgacgcagagcagcgccctggccgggccaagcaggagccggcatcatggattccttcaaggtagtgctggaggggccagcaccttggggcttccggctgcaagggggcaaggacttcaatgtgcccctctccatttcccggctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcagggcccagccggttcagagcaaaccgcagaaggcctccgcccccgccgcggaccctccgcggtacacctttgcacccagcgtctccctcaacaagacggcccggccctttggggcgcccccgcccgctgacagcgccccgcaacagaatgggtgcagacccctgacaaacagccgctccgaccgctggtcccagatgccagcaagcagcggctgatggagaacacagaggactggcggccgcggccggggacaggccagtcgcgttccttccgcatccttgcccacctcacaggcaccgagttcatgcaagacccggatgaggagcacctgaagaaatcaagccaggtgcccaggacagaagccccagccccagcctcatctacaccccaggagccctggcctggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggggccgctacctggtggcgttgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgagatcatgcacgccctgaagatgacctggcacgtgcactgctttacctgtgctgcctgcaagacgcccatccggaacagggccttctacatggaggagggcgtgccctattgcgagcgagactatgagaagatgtttggcacgaaatgccatggctgtgacttcaagatcgacgctggggaccgcttcctggaggccctgggcttcagctggcatgacacctgcttcgtctgtgcgatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctgggctcccgggccctgccca

SEQ ID NO: 40 - protein, 153 aa, Homo sapiens
MDSFKVVLEGPAPWGFRLQGGKDFNVPLSISRLTPGGKAAQAGVAVGDWVLSIDGENAGSLTHIEAQNKIRACGERLSLGLSRAQPVQSKPQKASAPAADPPRYTFAPSVSLNKTARPFGAPPPADSAPQQNGCRPLTNSRSDRWSQMPASSG

SEQ ID NO: 41 - DNA, 24740 bp, Homo sapiens
  unsure 1..6: a or c or g or t
  unsure 8101: a or c or g or t
nnnnnntgtattttatcatattttaaaaatcaaaaaacaaaaggcagttgaggttaggcatggaggttcgtgcctgtaatcccagcactttgggaagccgaagcacgtggatcacctgaggtcaggagttcgagaccagcctgcccaatatggtaaaaccctgtctctactaaaaatacaaaaaattagccaggcatggtggtgggcacctgtaatcccagctacttgggagactgaggcaggagaatcacttaaacccgggaggcgggctgggcgcggtggctcatgcctgtaatcccagcactttgggaggccgagacaggcggatcatgaggtcaggagatcgagatcatcctggctaacatggtgaaaccccatctctactaaaaatacaaaaaaaattagccaggcctggtggcgggcacctgtagtcccagctacttgggaggctgaggcaggagaatggcgtgaacctgggaggcggcgttgcagtgagccaagatcgcgccactgcactccagcctgggcgacaagagtgagactccatcttaaagaaaaaaaacaaacccgggaggcggaaattgcagtcagccgagatctcgccattgcactcaagtatgggtgacagagcaagactccatgtcaaaaaaaaaggcagttgacaggagcaaggagcctggtgaggaagctgtggcatttgacccggctgtgttgctatgggccagggtggtgctagtagaggagctgagtgggaaagagcacaggggacatgctgaaggcctgggtgtggggatgaggcagagattgggggcaccttgcagggtcatagcaggtggctgtggtgagatggaggaagacacctggggtactgctctaggctgtcagacatacagaagctggcccagccaagcccaggggctgcaagggacatccttttgtgtccccagtgatctgcagctctcagacaccctcaagcacagtgcctcttgcccagcccagcactctcagtggggagccaggtgggagaacaggctcggaaggggacctaggcttatgcagcgagccgggcaaagctggaactggagcccaggcccctggatgccccctggcttgtggagttctgggatactgaggggaggggacagggcatgggagtgcggtgctctcacctttgacttgaactcattccccaggggacaggggaggcctcctcaggatccacagatgcccagtctcccaagaggggcctggtccccatggaggaaaactccatctactcctcctggcaggaaggtaagttggaggacgtgcaagggcagcctcagccccccacacccagggctgggtctttttgggactgacggagctgtcctggccacctgccacagtgggcgagtttcccgtggtggtgcagaggactgaggccgccacccgctgccagctgaaggggccggccctgctggtgctgggcccagacgccatccagctgagggaggccaaggcacccaggccctctacagctggccctaccacttcctgcgcaagttcggctccgacaaggtgaggtgcaggggtgggaaagggtgaggggctgacagcctggaccctcctgctaatccccacccgtgtgccctgtgcccagggcgtgttctcctttgaggccggccgtcgctgccactcgggtgagggcctctttgccttcagcaccccctgtgcccctgacctgtgcagggctgtggccggggccatcgccgccagcgggagcggctgccagagctgaccaggccccagccctgccccctgccacgggccacctctctgccctccctggacacccccggagagcttcgggagatgccaccaggacctgagccacccacgtccaggaaaatgcacctggccgagcccggaccccagagcctgccgctactgctaggcccggagcccaacgatctggcgtccgggctctacgcttcagtgtgcaagcgtgccagtgggcccccaggcaatgagcacctctatgagaacctgtgtgtgctggaggccagccccacgctgcacggtggggaacctgagccgcacgagggccccggcagccgcagccccacaaccagtcccatctaccacaacggccaggacttgagctggcccggcccggccaacgacagtaccctggaggcccagtaccggcggctgctggagctggatcaggtggagggcacaggccgccctgaccctcaggcaggtttcaaggccaagctggtgaccctgctgagtcgtgagcggaggaagggcccagccccttgtgaccggccctgaacgcccagcagagtggtggccagaggggagaggtgctccccctgggacaggagggtgggctggtgggcaaacattgggcccatgcagacacacgcctgtgtccaccctggcctgcaggaacaaggcaggccgcctgtggaggacctcagccctgccctgccctcctcatgaatagtgtgcagactcacagataataaagctcagagcagctcccggcaggggcactcacggcacacgcccctgcccacgttcattgcggccaacacaagcaccctgtgccggttccaggggcacaggtgacctgggccttacctgccacccgtgggctcaaacccactgcagcagacagacgggatggaaatcattaggactccatgttgctctgcacggccgagtgacacgaagaggcaggcggagggagctgtgaggcttacttgtcagactcaggaaggagcaacatgagggcccaactggagacccggaggcccgagctgggaggaggcagtgggggcggggtgcaggtggaagggatttcagagacaccctcgtccaaaacacttgttccctgctgaaactccaacaatttgcagatacttctgggaaccccaggcgtcagtctcctcatctgtaaaggagagagaaccgatgacgtatcaggcataatccttgatgagagtttgctgcgtgcctactcagtgccaggcgctgggggacacagccgtgttcaggacagccttggtcctgttctccgggagccgacattccagggggagagaagtttcctgaagacttccatgctgcgttccctcctctgctcctgctcctggcgccatcctaggagccagccatgcacgcaagcgtcatgcctccagggctctgactgcccagcccctcaccgcaactccacctcagctgcacacacccttggcacatcctgaacctcattttcatgacggacacacaatttttgctctctcctgtccaagcctcatcctctggccgccacctccttccagctcacttcctttagtgcggccagtaccgcccctgcctaggcatgtcgacctgcagggacccttttctggctcttcgaggcctctgcccaccatcccctctttgttctccatagtcccttccccctgttctctctcgtttcatcttactggtctggcaaagtccccggccttgggcgagccagacctcctcagtgcctgcacacagctgcccacagccagagaaatccatttaagcagactgcctgcatccttcttaacagtgcaaggcaggcactccctgccacaagagaccctgttccctagtagggcagcttttctcctccccagaacctcctgtctatccccacccaatgtctcctcacaggcatattggggaaacaggtcaggctctcccaccgtatctgcaagtgtactggcatccatctgtcttcttcctacccctacagtagaaacagtgtctgtccccagctgtgctctgatcccggctcctttcacctcagagcttggaaaattgagctgtccccactctctcctgcgcccattcatcctaccagcagcttttccagccacacgcaaacatgctctgtaatttcacattttaaaccttcccttgacctcacattcctcttcggccacctctgtttctctgttcctcttcacagcaaaaactgttcaaaagagttgttgattactttcatttccactttctcacccccattctctcctcaattaactctccttcatccccatgatgccattatgtggcttttattagagtcaccaaccttattctccaaaacaaaagcaacaaggactttgacttctcagcagcactcagctctggttcttgaaacacccccgttacttgctattcctcctacctcataacaatctccttcccagcctctactgctgccttctctgagttcttcccagggtcctaggctcagatgtagtgtagctcaaccctgctacacaaagaatctcctgaaagcctgtaaaaatgtccatgcatgttctgtgagtgatctaccaagaaaataaaaaattttaaaaatcaaatgcccatgcctgggcccacacgcaggggctctgatttcatcagtctggtaggtgggttctgggcatccacgctcactggatttccggatgattgtagtatgcagcctaggctgggaaccactggcctcagcaagccagtcattctccaggtgtcacagaccctctaggtgctaatgaccccgaaggtctgtcttcagtgcacacctccccctgagctccagatttaggaatcccactgcacacgagacatctggatgtggaaaagacatctccagatcccatgggtgaaagggggttgggggaatggagactcgtgttcttccaggatgtgtgtggacacagaatgcaaagcctggagggatgctagagccatagggaggaagatttcggctcacttattcatgcaagcacttcctgatgggtaaggtcttagagcaagctgaggccaagaggcgggcagtcgaggtgctgctgcaggcacccccactccctacagtggcaagcccaagcccagcccttggcagctcaaatcccaggacacgctgaaggtcacccagagagtcaggggcatggctagaaccagaacccaggactctggggacccagcatggcatcctttccttcattacaaatctgagctgctttgtttcctagggatttctgtgatattccaaggggactgtgggaaagaaagtccttggaaaccaccaggacgctagaggcctggcctggagcctcaggagtctcggccaccagagggcgctgggtccttgtccaggtccagttgctacgcaggggctgcctgtgctgggaggctccccaggggacacagaccagagccttgcaccagcccaaggaatgggagcctggggtcctctctgctggaggactgccaggacccccaggctgccgcctcttcctttgctcatttgctgtttcactttgtcaatccttcctttcttcgtgtgttcattcacatccactgtgtgctggccctggggaaatgttagataagacacattagctgtgtgtcttcattgtcctaacaaagaacacaccctggaaagagcaccgcagagagtccccattcccccatctccctccacacatggaatctggagatgccttttccacatccagatgtctctggtgctgtgggattcttaaataaacaaacatttcatacagaatgtgagatgatggagatgctatggggaaaagtaaagcagagggagggcctagtgtgtgatgcgggtgaggcatccagggattgctgtttcagctgtgatcaggaaaggccctgggaggaggccacatctgagcagagacctaaataaagttggaaacctgttgctgagatatctggagaagtgtttcaagggccgggcaccgggcatggtggctcacgcctgtaatcccagcactttgggaggccaaggcaggtggatcgctggaggtcaggagtttgagagcagcctgaccaacatggagaaaccccatctctactaaacatataaaaattatccgggcatggtggttcatgcctgtagtcccagctactcgggaggttgaggcaggagaatcacttgaacgtgggaggcagaggttgcagcaagccgagatcacaccactgcactccagcctggatgacagagcgagactccgtctcaaaaaaaaaaaagaaaagaaaaaagaaaaaaaaagaaaagtgtttcaagcaggggaactggcaagtggagaggccctgaggcagaaatatgcttggcctgctggaggaaatgtgagtgaggaggtcagggtggctggagtggagggagcgagtggtaggagtcagacccagtttattcatattctgtaggtcttaaggacttcagttttattttgagtgcaatatgagcccactggaatgctaaaagctgagagtgacatggtgctgtgattctggctttaaaaatatcactttggctgcttcgtgaagactctggaaggggcaagggtgaaagcagggatgcccgttaggagaccgttacaggggcgcaggcacaaaatggcagtggctgggacaatggtggcagcagcggttagatgtgaacatgttgaaggtggaatttgcagaatctgggggaggacagaagagaaaggataacttcatcgtttctgctgaaccagttggataaatgttggtggcacttcttgaagtgaggaaggagttaggaaggtgggaaaggcacaagtttgaattgggccatgatggtctgagatacctagtacagtggttccccaacctttttggcagaagggaccgctttcatggaagacaatttttccacagactgggggtggggtggggatggtttcagggtggttcgagtgcagtacatttatcattagactctttttttttttttttttttgagatagagtctcgctctgtcacccacactggagtgcagtggagccatcttggctcactacaacctctgctgcccaggttcaagtcattctcctgcctcagcctctcaagtagctgggattataggcatatgcgccaccacgcccagctaatttttgtatttttagtagagacggggtttcaccatattggccaggatggtctcgaactcctgacctcaagtgatcctcccccgcctcaacctcccaaagtgctggggttacaggcgtgaaccactgcacccggcccatttatcattagattctcataaggaatgagcaacctagatccctcgcatgcacagttcacaatagggttcacgctcctatgggagtctaatgctgccgctgcactcagcttctctggcttgccgctgctcaccttctgctgtgcagcccagttcctaacaggccacaaacggggagttggggacccctgatctagtaaacatctaggcagggttttggataatggagttagagttcctggggagaggtcaggctggccatgaaacatgggatgcctttgcatataggtggtgttgaaagccacaggacagtacggggtctcagggggtgagcataaagagaggcgacatcagatggccaaggccagaggcagaggaggatgggaaggaggggccagtggggcagggggaagctgtgaagccagggaaaaagggtgtttcgcggaaaaggatcaacctggaccagtgctgcccctaggcagggcaggatgaaacttaaccaccacggattccatggccccatggcctccaggccacaggggaccttgagaagagagatctcaggggacgggtgcggacaagagcccgcctggcatggcttcaagagataactgaaggaaagcaagtggagacgcgataaacagacaactccctggaggaattttactctcgagaggagaattaaagggtagtagctggagagggatgtggggtcaagagaaggtctttaacgacgagaactctcacggcggtttgtgcagaacagggtgggtgtgatgactgtggatggagaggggagaactgcagcgactctgtcctaggaggaggtgatgggccgggaccaccaagcgagtggagggtggacgccccttccctcaccccgacacccgcatgtgctcagtgtccgtgccgccggccctagtgcctgggctgaacgcggggccgggactctgaggacgcctcccaggcgcgcagtccgtctggccaaggtggagcgggacggcngcttccgacggtgcgcgggtcggctcggggttgcagggacatccggcgtccgctcctgccctgttttcctgccttcgcagagcgttgcgcaactctagctttaaacgcccctgtccccctcaacttgtctcccccagcccctctgatttacagattctgcagtccccgagggttgcgcctacgataccgacactcgcggcagcctgcgaggcgagtatgatcgtcccatttttcggagtagcaaactaaggttcagagactactatgtcccaggtcggtctggtttgaaggtccgctttcctctccctccgccagcgggcggtgcgagggactgggcgaggcagcgcttccctaaggaggcgacccgcagccccggccccctcccgactccgccccgttgcagggcccgggtcggcgaggcctctcagctctaagcccgacgggacttggtgattgggcaggacggaagagctgggtggggctttccaccagcggagaaagtctagtgggcgtggtcgcgacgagggcgtggcctggtgccccgcccccgtccgcgcgctcaaagtggagggtggctgtgggggcggggtcagaacactggcggccgatcccaacgaggctccctggagcccgacgcagagcagcgccctggccgggccaagcaggtatcgacgaccgcgcggggcgtcttgggctggaccaggcgggcgcccggggcctgctgaggaccacaaagggcactgggggtcgtggtccaggctgtgcttcctcccgctggccctggcccctgcctccgcccccgcccccgccttcctgccgctaagccggctgcggcggggccgattggcgcctgccggcttcctgcgccggggccagtctaatgcatggggcccgggcgggggactaaggggaaactgagtcacgtcggtgtgggagcagttctgtgtgggaggcaccaccccccactgggctcggggaaggatccccctccaagctatgcttgagggtcccagcccccatctgtctccacaggggccgcaccccactcccgccttccccttcttcagcacccaggggtcccgccctggctcccagcagcctcgactggtcccggaatggctaggaggatccgctgcagccgcctccctcccctcccctcccctcccctcccctcccctcccctcccctcccctcccctccccctcgcgtcccaagcccccgtgtgctccctccgctggctctccgcacagtgtcagcttacacgccttatatagtccgagcaggctccagccgcggcctgctgccgggacctgggggcgggggagaggagagccggcccctgactcacccggaccgcccgaggctccaggctggcttggggggaggccgcgccagtttagtccctcggcccacccctggttgcaaagaacctcaagcctggattcaggcacccctcaccgttccagtcccaaggggaggggggctgctcctgtctttccaaagtgaggtccgccagccagcagcccaggccagcctgacaaaatacctgcctcctatggcttgggcgtgctcaggggctgcccgtgcctgcctggcccctgtccaaggctggtatcctgagctggcccggcctgcctgcctgcccgcccaccatgctggccactcaccttctcttctctcctctcaggagccggcatcatggattccttcaaagtagtgctggaggggccagcaccttggggcttccggctgcaagggggcaaggacttcaatgtgcccctctccatttcccgggtgagcctaggtttggggagggggctcccccagcggtctttcggtgcttaggtctccagagggtgatggggggagtcctaacaggagctggtcaggggccagcaggccaggagatgtctaggtccggagatgtagtggtacctgcctgccacaaggactcccaatgaggtggatactgggagggagcacccaggcttctccagccctgcactgtacccgatgctgttctcccaagctcctgtggccacctctgagggctggagggaggctcattgtgcaggatgggagcctaacatttcaggaggtatctaaacttgaggtggcaatgcttggagccaggccccaggcaggacactgtgactataggatttcacttcagcctcactgccgcccagggaatagcaatcctcatcccgtttttccagatgagagaagaactcatggagaggtggcggggctcgctcatcgagtccatggtgaagcagggattggaattgaggcacagcatggcgtacattttttgtgggtagaaggggtctctccccagcctatgtaaggacccacatccactgttcccattcaggatgtggtggcctttgaccccaagcagaagtgtaggacagggctccattctaggggcttaacttcagcttccaagagcctgccctggtgtgggtggagctggaggctggctcctccctgtagcagggggattgccttataagcccaagaatgcagccccacgctgggatggccaacagtggctgcggtctgcagagctgaaaagggctggcctaggcctggccccctgaaccccactggtgggcctctcagctggtcaccaggctgcagctccagctgtatggtccagttgtgagacacaacaaattgcctgcccagagtgggtgaggccagcctgtcggctggcatctctgactggcctgggggtcaggagggggtggggacttcctgcccctatatccgcctgccccgagagacccacccaggcgccgggtgggcaggcagctgttgtcaggaagcccaaggcaagcccagcctggaggggcccagagggtcgtggcctgaggaggggctcaagctggagtctgtctgtaggagctgggcgtgggggttagggtgggcaggccagcagtgctcttctcaggggtcctttgatggcattctcctggaacctgccccgccagcagggtagtgaggcagtggttgccctatgacacacgtcccactacatagccctcacacagccctgaaacctacctgacgtcctgctccctgggaaagtgctggcccagtgtgtctggggagcctgaacctcagtttcttccctgatggagatgactttcagatatggcctgttgggggcactccgggctccagctccctggtcagcatccctggcatgtgggcggggccactagctgatcccagccctggagttggacctgggcccacatgggtgggtgaggtgggcttttctgagttaggccagccccctccccctcccctgaccccagaatggagggaggtgggaggggcaagggctggctgtgggcccaggcctgggagatgaggtaacgtctgggactggggggctgggctgctcaggctgactcacccccacctcatgcagggtccagccccctggctttttccctccttggttcctctggccttaccctgcccctggcttgagcccctccctgcctctctccagccacccgcccagcgctgtcttctgctctcctgctgccctccccacgctctgaacacccctcatcctctgtgcttcctgccctcctcactctgggaagggaagccgtccccgccccccaccccctctccaggagccagctagctgcaccccaagacccccacctcgggctcagcccacagctcccaggagccagccctgtgggcagggagtggctgggccaggtttcccttctactgactcaccatgaccttgagtaagtcacttcccctctggggtgtcacttccccatacacagtataaggggttgatttagttggattgaactaaaggtgagggagtggctcagggtgtctccaggtgggctgacccctcagttgggcccccatgctcagcagaggtggcccacagtggtggagccttagggtcagagacacttcctggctctgcctcttactagctgggtgacttgaggcaagttgtttaacctctctgtgtacatttgcaagtgcaaaatgggtaaaatcccagattactccacaaggttgttggaagattcagtgtcaatatgtagcatagttggtgctcaataaactgaagcaagtcttcttatttagcgagtgaggaaggggccgccgagctctcttagccttctgacctcctacgcaagcaagaggtcatgttgagcccagctcgcctttcttttcccagtgctgtcaagctctgtgcctggctgccctgccctctgacatctctctgaaacctcttgcctcccctctccctgcctcagctcagtctgtgcactgacccacctgaggagcctcctggggccactggcagcctggacccccccagatcccccccacccagtgaaattgtcttccagcactgcctcacaaaagcctacttgatgcagtgccaggcctcttgccagatggctgggtggtcccttaggcttggacccagtcaagctgccctgcctgtgttgctggggctgggctagaggcctggaaggggtttatcagggtcaccctctcagggcctgggagatacccaatcccagacattaaaactgccagtagcccctctaccttcaaagccaagtcctggtcccttcccctggcattcaaagccatcgtaagtgaactctcacccgctaggcagcacacgccattctcctttaccgaggcccaccgcttcctcaaagtcattcctgatggtctcagctcatgctggtggcagccatttctcccagcctactgtctctactcattgccacaggaaccagggactcccagctcaagagcctgaaggattggggtcaggggaaattggcagtcgagggcttgggagtgacagccatgtatggcctacgaagtcccagctgtcaacttaggtcccattcaggcagtgttcacagggaaccgggagataacagggcctgttcctggctctcaaagggtcccagcagacccctatagatggcccccgacagggtgctggggggtgagaggtccataagagcccccggtggtttcggggaggaagctgccccctgcatgggccagagggcatatctggtaggtggagtggcctgggcaggaggccagcaggagcctcaaaaggcaatggtcctcctgaaacacttgggctttagcctgagcgtggctgtttgtggacatcatagcaatttctggactgtgggggagggtggtggcggtgaatagataagcatcgtgactggggaagctcaggtgagcaccacctgagggagagggtctggcagtgaataaataagcagtgtgactgggaaattgtgaagctcaggtgagcgccaccacctcctgggttgctttagtgtccagcagctgcctagaactatgttgaatgaagagctctctgggttctggaagtgggacagctttgggtggggcagtgttaccaccgtcagcctggcttgggtctgcagggtccagggcctcggtcactttgcttctctctccacagctcactcctgggggcaaagcggcgcaggccggagtggccgtgggtgactgggtgctgagcatcgatggcgagaatgcgggtagcctcacacacatcgaagctcagaacaagatccgggcctgcggggagcgcctcagcctgggcctcagcaggtatgcgggtggacatggatgggtgcgcccgcgctggcagtggggatccctgcggcccggcccgctgtcacgctttccttctcctccagggcccagccggttcagagcaaaccgcagaaggtacgaggctggccgggacatccgggcggtgggcggtgtgggcttggacggccaggcctgctcgccctcctggcacattctcggtaccccaatccctggccgggagtggagggcagaaaccggagctaaggcgggtctagggccctggagttgagccaggggctgctgcacggtcctggcaccacgcatgtccgcctgtctgtccgcctgtctgtccgcctgctgcctcccgccgccggcgctgcgtgctcgcccgcactcggtcagccctcggtcctgcgtggactgagatcgccactcccaaatgggccccttgaaacctgagtcgtcctctccccgtagcctccaaatagatgtagggggtggggtgggggtggggggctggagctgccgctgtcctctgctgcaggcgccccacttccacccaggcccccaccttaccctgcccgcccgccctgcccggctgtgtctctgcccaggcctccgcccccgccgcggaccctccgcggtacacctttgcacccagcgtctccctcaacaagacggcccggcctttgggcgcccccgcccgctgacagcgccccgcagcagaatgggtacgtcggcccctgcccgcccgcgcccacgccatcaggcccactgtggccccacgcccgctgcccgctgctgctcagtctgtgctgcgccccagcccggcggaaccgtgcggcacgccccctggcggccggggtggggctgcaggcacagggcccctcccgaggctgtggcgccttgcagggcaccgcctggggaggggtctctgaatgacgccgcgccccctgctggcggctgggggttgggttgtggtgtcgggccagctgagccccagacactcagtgccgccttgtccccggctgttctgacccctccccgtctttcttcctctcctgtgtctgtccctttgtccctttatctgtctgtctgtcttatttccttcacaggtgcagacccctgacaagtcagtgagcccccctctgcctgtgcctttcttcttccttttggcactctgggtggcggcccctccccaccctggctgccctcctctccacttcgccctcctgtcctctcacctacccgcccagcagggctcctggcctcacccttacccactccctcccatcactgtaacccaaacccacatgcaccaaatcctgggaggggctgcccccaccgcccacccccagtgtggggttctgagccacaccctccccacagacagccgctccgaccgctggtcccagatgccagcaagcagcggctgatggagaacacagaggactggcggccgcggccggggacaggccagtcgcgttccttccgcatccttgcccacctcacaggcaccgagttcagtaagtgccagcccagggcagggggtactttcctcgcccccagcccaggcgtgatccctgaccctgtgtcttttttggtcaatgcctgcctctgctctctcagtgcaagacccggatgaggagcacctgaagaaatcaaggtacagggacgggcaccagcccctctcccacctcctgcctcttccattccagctactgccctgtgtctactcctgaggctcccagctggggctctcaattctcccttccttccttccttccttccttccttccttccttccttccttccttccttccttccttcccttcctccttccttccttctttcatttcttccctccctccttccttccctcctccctccctgcctcccttccatctctccttccttccacttcttcctccctctctctctgcccctcagggaaaagtatgtcctggagctgcagagcccacgctacacccgcctccgggactggcaccaccagcgctctgcccacgtgctcaacgtgcagtcgtagcccggccctctccagccggctgccctctctgcctccctctttctgttcctcctgcccagggcacccccttagtgcctccagcttctgcctacctcaccccccctttcgtgcccctggcctgagcctcctgctggcctggccctggccgcccacctgggttcatctgacactgccttccctctttgccctgtggtactgctgtctgccaggtctgtgctgccttgggcatggaataaacattctcagccctgcttgctctgcctgtcttctatctttgtggacctggtttgcatttggggtgtgggggtgtttcgtggttcggactgtttgggccctgccgtccttgttttcagtgggagggggtacctggcaaaggggccctgccctgccatcacagatggcttcctggcatgaggggagccccaggagctgcctcagaagcgggagccctgcctcgtctcccagctagagaccgcacaccagctaactggacattgctaggagaagctgcccttcccatccctaccccagtgggacctggaatccaactcggcagtttccacgcccccagtcatctcccgtggggccagcaggacccaggttggggggtggggccatgtcaggaagctcagccatgcagggccttgaatggcagatcttgcagccaggtgcccaggacagaagccccagccccagcctcatctacaccccaggagccctggcctggtgagagggagtgggctcgggcctgggcaagggtgggcagcctccaggggcatgggggtggtgggcttctctcagctgcctggggctccacccccgtcctttggggtccctgggcacccctttagagtcactttccccggcaggccctaccgcccccagccctaccagccgcccgccctgggctgtggaccctgcgtttgccgagcgctatgccccggacaaaacgagcacagtgctgacccggcacagccagccggccacgcccacgccgctgcagagccgcacctccattgtgcaggcagctgccggaggggtgccaggagggggcagcaacaacggcaagactcccgtgtgtcaccagtgccacaaggtcatccggtgggtggcctgttcctgtccgaccctggctttcccatcctgcagcccagccccacctgtctgcccacctgtcttgcctcagctgcgactggggggaataaggattcagttctcagctggagtaggagtagggacctgggctgggtcctcccattcttaatcccacgctacctaccccagcccacccacaacaactgctagcagcatctgccgtggcgaaatagccgaagggccaaccataggctgaagctgcacccctacctttgctgctctctgggcaaagaggggcctgccccctcccagcgcgtctgcccctccctcctgctctctgtctccctctgctctcagagcatacaggcctggagccactccctctgtgcactgccccgtggggccaagcagcatcaaacaccccccagcatcagcgtgccggattctagagccttcctaattcgcaggcctggcctgctctcatctctgtcagctcttttttttttttttttgaaacagagtctcactgtgttgcccacgttggcgtgcagtggcgcgatctcggctcactgcaacctctgcctcctgggttcaagagattctcctgcctcagcctcctgagtagctgggattacaggcacccgccaccatgcctggctaattttgtatttttagtagagacggggttttaccatgttggccaggctggtctcaaactcctcacctcaggtgatctcaggcctgccttggcctcccaaagtgctgggactacaggtgtgagccactgtgcccagccgactctatcagctcttgccaggtagaacaggcaggccagcaggacagggcagctccagggtttgcccaggggcggctcagcttttatgaggctccagtcgtcagcccttcctcccggggtcctccctgctctaaagctgcctctcctgtcaccagcagttcagtgtggcggactggctctgtaagcttcatggctgccacggtcacttcccaagcctgtcttctatcctatgtggaaaatggggagaatgaactgtccctcccaaggcctcctggtgggtggtcagtcaacctgaagggggccaagacccccacctctctgcgtgtgctccctctgaccgctctcgcctccctgcaggggccgctacctggtggcgctgggccacgcgtaccacccggaggagtttgtgtgtagccagtgtgggaaggtcctggaagagggtggcttctttgaggagaagggcgccatcttctgcccaccatgctatgacgtgcgctatgcacccagctgtgccaagtgcaagaagaagattacaggcgtgagtagggctggctggcggggaggtggtcccaagcctgtcagtgggaacgagggctgctgggaaacccacagtccaggtctctccccgagtgagcctccgggtccttaccagcgtaataaatgggctgctgtactggcctcaccctgcattagtcaggatgctcttaacaaatgaccatgttcctgctcagaaaccgcccaaggctgcaaagagcaggaggaccaagccaggagaagccctgggccctcctgactcccactttgggctctccctgccctggtgaaatgacagaacggccaacttgacacgctgaagctgctctgtctcatgcgtcctcctcatttctggatccagagccagggctgccaggagtagccagagagctctgtgtggtgatgttcatattagtgaggtttaccttgaccacgagcagtgggaaactcaaaataatggtggcttatttctcatctaaaaacatcccggggtgggtggtctgggactgatctggtggacccaggctccgccttgttgcttgactgttggcagcacctgcttacttaccactcatggtgcaagatgacacttcagcctccgccaaaatgctcaccttccagccagcaggaagtcggaaggagaagaaaggggacagagccccatggcgtccatccttagaggatgctgccacctgaacctctgctttcatcctgttggtcagaacccagtcacatgaccacacccagtggcaacggaggctgggaaatatagtctttattttgggcacccatgtgtccagcaaaactgggggttccatcagtcggcaagaacgggagagtggccgatgcagtggctgatgcttgtatcccagcactttgggaggtcgaggtgggcagatcacctgaggtcaggagttcaagaccagcctggccaatatggtgaaaccctgtctctactaaaaataaaaaaattagctgggtgtgctggcgcacctgtagtcccagctacttgggaggctgaggcaggagaatcgcttgatcttgagaggtggaggttgcagtgagccaagattgtgccactgccttccagcctgggagacagcaaaaaaaaaaaaaaaaaaaaaaaaaaaaagggccaggcacggtggctcacacctgtaatcccagcactttgggaggccgagatgggcggatcacgaggtcaggagattgagaccatcctggctaacacggtgaaaccccatctctactaaaaatacaaaaaaattggccgggcatggtggagtagtcccagctactcgggaggctgaggcaggagaatggcgtgaacctgggaggcagagcttgcagtgagccgagatcgcgccactgcactccagcctgggcaacagagcgagactcttgtctcaaaaagaaaaaaagaaagagaaatctgcctcccagccttgggctcctgccctaccagcccacacccctggtagagcctcctctcccaccagctcaaagcccaagttccttcactgtgaccttgtctgctcctctaaaacaggcaacaccagacagtgagaagagccagccagacatgggcagaaaacctatttctgtgatctactggctgtgtgagcaggggctagttgctctctctgggcctcactgaagagaagggtggcactatgctagggccggcacggttgcaaggtagatgtaagatggggtacaggtgttgtggagggcagaaatgcaccatccgaaggctacatgtcccccacacttatgtcttgcttggcccacactgtttcattttaaaatcagtagcaaacaatttaaaaaatcagaagatttgcctgcatgatgcagtggctcatgcctgtaatcccagcactttgggaggccaaggtgggaggattgcttgagcccaggagttcaagaccagcatgggcaccatagcaagacccctgtttctacaaaaaaaaaaaaattagaaaattagccaagtgtggtggcatgcacctgtggtcccagctacttgggaggcagagggaaagtgagatctcctgctttttatttctttatgtataatgatagggtcttgctctgttgcccaggctggagtgcagtggcatgatcactgctcactgcagccttgatctcctgggctcagaggatcctcccacctcagcctcccaaatagctaggactagaggtgcccaccagcatgctcagcagatttttaaatctttttgtagagatgaggttttgctatgttgcccaggctggtctcgaactcctggcctcgagcgatcctcccaccttggcctcccaaagcactgggattacagacgtgagccactgcgcccagcagatttctctttaacacctagatttcagcctgagccaggcaggcattcctgaatgaaccagtagtactgctcccagaagaagaggtcctcctccgtgtgacacagtccccacttggcccttgcagggattggatctgggatccctggatttaaactcagggccatcctcataacagcctcacaaggctgggattagcttcccagttcacaagggaagaaaccaagacttgagaaggtcaaggtctggccagacccacacatcttggaccctcataccgcctcgaggccccatgctgccctctgcctgctccagatgtgaatactgctggccctggctggccccggctggccccgagggtcctagggatgaacagcccagcccagggagagctcagccccttgtgcctctgccccttcccacctcctgcggaggccagtcgactcacccacaaagggccaggcactgtggggatagatcagctaacaaaacagttgatgcttcctgcccttctgggccttacattttggctggaagaagaggggagaggcagactgtaagcaataagcgcaataagtaggttgcctggaagtaatgttagatcacgttacggaaaacaggaaagagcagagcgacaagtgctggggtgcgtggtgcagggaaggcagctggctgctgctggtgtggtcagagtgggccctcatggagaagactgcattcgagcagaaacttgaagggggtgaggggtgagcctagagatatctggggcagagcagtccaggcagaggggacagccggtgtcaagcccaggacaggagtgtgcctggtgtgccagtttcaggcaagaggccagtgtgcagaggcaaggtgagaacgcaagggagagcagtggcggagacgggtgggaacgaggtcagacctgctggcctccagcctctgcatggggcttggctcttgctgggagcaatgggaagcagtacacagtttcatgcagggggagaaggcctgtcttgggttgcaggggcacgctgtggcagctgggatcagagagaggagcttgtaggccagttgttatgtggtcccacgggccagatggccatggcttacctcacttcagggaggctgtgagaagcactcagaatctggatgtgccttgggggtgggccccactggatttcctggtggacctggtgtggggtgtgagaggagggtgtgtttggctgcagcagacaggagaatggagttgccatccgcgtgatggggatggctgtgggaggagaggtttggggtgagggaatcaggaactgagtgctggacatggcaagtctgaaggcgcagtggtcgtccactcagagaccttggagttggagatggaggtgtgggagtcctgaacagttagatgtagtgtttaccgcgagaaggaacagggcttgcggccagccctcctgtgttcccgtgacccagggcagggcaggaggggcctgagcctgccgagtgactgggacctccttccaggagatcatgcacgccctgaagatgacctggcacgtgcactgctttacctgtgctgcctgcaagacgcccatccggaacagggccttctacatggaggagggcgtgccctattgcgagcgaggtacccactggccagtgagggtgaggagggatggtgcatggggcaggcatgaatccaggtcctctttctctctgcccccattctcagactatgagaagatgtttggcacgaaatgccatggctgtgacttcaagatcgacgctggggaccgcttcctggaggccctgggcttcagctggcatgacacctgcttcgtctgtgcggtgagagccccgcccctcgaactgagccccaagcccaccggccctctgttcattccccaggagatgcaggagaagttgggaaggggcctctcctgctgcccccaaccccatgtgactgggcctttgctgtccttagatatgtcagatcaacctggaaggaaagaccttctactccaagaaggacaggcctctctgcaagagccatgccttctctcatgtgtgagccccttctgcccacagctgccgcggtggcccctagcctgaggggcctggagtcgtggccctgcatttctgggtagggctggcaatggttgccttaaccctggctcctggcccgagcctggggctccctgggccctgccccacccaccttatcctcccaccccactccctccaccaccacagcacaccgatgctggccacaccagccccctttcacctccagtgccacaataaacctgtacccagctgtgtcttgtgtgcccttcccctgtgcatccggaggggcagaatttgaggcacgtggcagggtggagagtaagatggttttcttgggctggccatctgggtggtcctcgtgatgcagacatggcgggctcatggttagtggaggaggtacaggcgagaccccatgtgccaggcccggtgcccacagacatgaggggagccactggtctggcctggcttggaggttagagaagggtagttaggaagggtagttagcatggtggctcatgcctgtgatcccagcactttggaaggccaaggtgggcagatcgcttgaggtcaggagttcgagacctcatggccaacacggtgaaacagcgtctctagtaaaaatacaaaaattagccgagtgtggtggggcatgcctgtaatcccagccactcaggaggctgaggcgggaaaatcacttgaacctgggaagtggaggttgcagtgagctgagatcacaccactgcgcgcgagcctgggtggcagatggcagagcgagaccctgcttcaaaaaaaaaaaaaaaaaaaaaaaaaagaagggtagttgtagttgggggtggatctgcagagatatggtgtggaaaacagcaatggccacagcaaagtcctggaggggccagctgccgtccaaacagaagaaggcagggctggagagggtagcccttaggtcctgggaagccacgagtgccaggcagtagagctggggctgtctcttgaggttagggcagggcaaggcacagcagagtttgaaataggtttgtgttgtattgcagaaaagaggccccagaacactgagggagtgcaggagggaggctgggaggaggagttgcagcagggcctaggggcgggggccaggcaagggaggggcagagagtaatatggcagagatgggacccagtggcaggtccgggggatgagggatggagagaaggacaggagcgttgccaggcatctggcctataccagacatgctcacgctgtctcccgcgaacctcctagcaaccttgcgccgttgtctgcaatcacttatttcattttttcttttttaactttaattttttttgtttttaagagacaggatctccctaggttgcccgggctggtttcaaactcctgggctcaagcaattcttcctccttagccccaaagtgctggcattacaggtgtgagccaccatgcctggcccacttattttctagatgaggcacagaaagattgggagacttgaccaaggtcacgctgtcattgagccatgagccagactagaatccaggcctgaagctgggtgcgctgtcccaggactggctggcactgagtaccatttgccagcgagcatctctctgggaagctgacttctgcccggtacctggaggactgtagaccttggtggtggcgccgtcactctggggcttcctgcctcccactgatgcccgcaccaccctagagggactgtcatctctcctgtcccaagcctggactggaaagactgaagagaagccttaagtaggccaggacagctcagtgtgccatggctgcccgtccttcagtggtccctggcatgaggacctgcaacacatctgttagtcttctcaacaggcccttggcccggtcccctttaagagacgagaagggctgggcacggtgactcacacctctaatcccagcactttggaaggctgaggctggagaagggctccagcttaggagttcaggaccagcctgggcaacatggtgagaccctgttttgttttgttttttgtttttttgagatggagtcttgctctgtcgcccaggctggagtgcag

SEQ ID NO: 42 - DNA, 25 bp, Rattus norvegicus
gcactaccttgaaggaatccatggt